(19) United States
(12) Patent Application Publication
(10) Pub. No.: US 2013/0150350 A1
(43) Pub. Date: Jun. 13, 2013

(54) Derivatives of 1-Phenyl-1,5-Dihydro-Benzo[B] [1,4]Diazepine-2,4-Dione as Inhibitors of HIV Replication

(71) Applicants: Bruno Simoneau, Laval (CA); Patrick Deroy, Laval (CA); Lee Fader, Laval (CA); Anne-Marie Faucher, Laval (CA); Alexander Gagnon, Laval (CA); Chantal Grand-Maitre, Laval (CA); Stephen Kawai, Laval (CA); Serge Landry, Laval (CA); Jean-Francois Mercier, Laval (CA); Jean Rancourt, Laval (CA)

(72) Inventors: Bruno Simoneau, Laval (CA); Patrick Deroy, Laval (CA); Lee Fader, Laval (CA); Anne-Marie Faucher, Laval (CA); Alexander Gagnon, Laval (CA); Chantal Grand-Maitre, Laval (CA); Stephen Kawai, Laval (CA); Serge Landry, Laval (CA); Jean-Francois Mercier, Laval (CA); Jean Rancourt, Laval (CA)

(73) Assignee: BOEHRINGER INGELHEIM INTERNATIONAL GMBH, Ingelheim am Rhein (DE)

(21) Appl. No.: 13/576,304

(22) PCT Filed: Feb. 9, 2011
(86) PCT No.: PCT/CA2011/050071

§ 371 Date: Oct. 19, 2012

Related U.S. Application Data
(60) Provisional application No. 61/305,108, filed on Feb. 16, 2010.

(51) Int. Cl. C07D 513/04; C07D 409/04; C07D 495/04; C07D 401/10; C07D 255/04; C07D 487/04; C07D 243/12; C07D 403/10

(52) U.S. Cl. CPC C07D 513/04; C07D 243/12; C07D 409/04; C07D 403/10; C07D 401/10; C07D 255/04; C07D 487/04; C07D 495/04
USPC 514/220; 540/518; 514/221; 540/501; 514/183; 514/338; 514/422; 514/406; 540/495

(57) ABSTRACT

Compounds of formula (I) wherein m, R¹, R², R³, X and Y are defined herein, are useful as inhibitors of HIV replication.

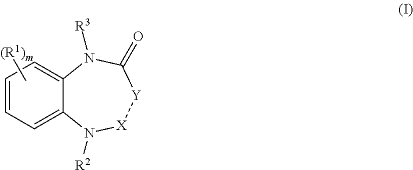

### RELATED APPLICATION

This application claims benefit of U.S. Ser. No. 61/305,108, filed Feb. 16, 2010, herein incorporated by reference.

### FIELD OF THE INVENTION

The present invention relates to compounds and their use as inhibitors of capsid assembly, pharmaceutical compositions containing such compounds and methods for using these compounds in the treatment of human immunodeficiency virus (HIV) infection.

### BACKGROUND OF THE INVENTION

To date, over 20 FDA-approved drugs make up the antiretroviral arsenal against HIV (1). These compounds generally target the viral enzymes or the viral entry process and can be divided within 6 mechanistic classes. The standard of care is a multi-drug therapeutic regime, often referred to as highly active antiretroviral therapy (HAART), where combinations of at least three drugs targeting the virus are administered. Although HAART can successfully restrict viral replication for many years, drug resistance can still occur. Cross-resistance within mechanistic classes and the emergence of multi-drug-resistant (MDR) isolates can have considerable impact on treatment options and patient outcome. This underlines the need to discover novel classes of HIV inhibitors.

The HIV-1 Capsid protein (CA), which plays an essential role in the viral replication cycle, may represent such a novel therapeutic target (2). CA is a domain of the GAG polyprotein, where it contributes some of the key protein-protein interactions required for the assembly of immature viral particles. During viral maturation, proteolytic cleavage of GAG releases CA which re-assembles to form a cone shaped structure called the core, enclosing the viral RNA genome and enzymatic activities required for infectivity. Core formation is driven by a multitude of weak protein/protein interactions (2). CA mutations that prevent core assembly result in non-infectious viral particles (3-5). CA-binding inhibitors of capsid assembly have been reported previously (6, 7), providing evidence that CA may be a viable drug target. Interestingly, the only inhibitors for which structural information is currently available seem to target the NTD-CTD interface, suggesting it may represent an inhibition ‘hot-spot’ (7, 8). References—(1) International Journal of Antimicrobial Agents 2009, 33(4), 307-320. (2) Current Opinion in Structural Biology 2008, 18, 203-17. (3) Journal of Virology 2002, 76, 5667-5677. (4) Journal of Virology 2004, 78, 2545-2552. (5) Journal of Virology 2003, 77, 5439-50. (6) Journal of Molecular Biology 2003, 327, 1013-20. (7) Nature Structural and Molecular Biology 2005, 12, 678-82. (8) Journal of Molecular Biology 2007, 373, 355-66.

Braccio et al. (European Journal of Medicinal Chemistry 2001, 36, 935-949) disclose the anti-HIV-1 activity for a series of nevirapine analogues, such as, pyrazolo[3,4-b][1,5]benzodiazepines. WO 94/17075 discloses diazepin derivatives for use in the treatment of HIV. U.S. Pat. No. 3,766,169 discloses benzodiazepine derivatives having tranquilizing and anticonvulsive properties. WO 2004/098610 discloses benzotriazepine derivatives and their use as gastrin and cholecystokinin receptor ligands.

### SUMMARY OF THE INVENTION

The present invention provides a novel series of compounds having inhibitory activity against HIV replication. The compounds of the present invention have inhibitory activity against HIV-1 capsid assembly. Further objects of this invention arise for the one skilled in the art from the following description and the examples.

One aspect of the invention provides compounds of formula (I) and an isomer, racemate, enantiomer or diastereomer thereof:

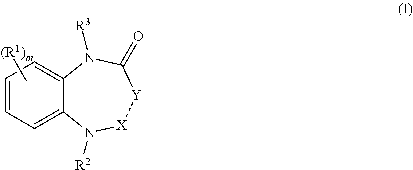

wherein
m is 1, 2 or 3;
either X---Y is selected from:

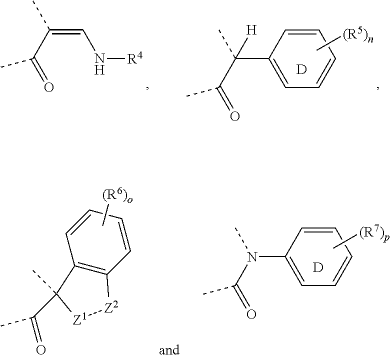

or X and Y are linked to form a 5-membered heteroaryl ring containing 1 to 3 heteroatoms independently selected from O, N and S, wherein said 5-membered heteroaryl ring is optionally substituted 1 to 2 times with substituents independently selected from (C₁₋₆)alkyl, (C₂₋₆)alkenyl, (C₂₋₆)alkynyl, (C₃₋₇)cycloalkyl, —(C₁₋₆)alkyl-(C₃₋₇)cycloalkyl, —(C₁₋₆)alkyl-Het, —(C₁₋₆)alkyl-aryl, aryl, —NH₂, COOH, CN, —C(═O)—(C₁₋₆)alkyl, —C(═O)—NH₂, —C(═O)—N((C₁₋₆)alkyl)₂ and —SO₂—(C₁₋₆)alkyl;
   wherein each said alkyl, aryl and Het, either alone or in combination with another radical is optionally substituted 1 to 2 times with substituents independently selected from (C₁₋₆)alkyl, —O—(C₁₋₆)alkyl, OH, CN, —COOH, halo, —(C₁₋₆)alkyl-Het, —(C₁₋₆)alkyl-aryl, —NH₂, —NH(C₁₋₆)alkyl, —N((C₁₋₆)alkyl)₂, —N(H)—C(═O)—O—(C₁₋₆)alkyl, —(C₁₋₆)alkyl-N(H)—(C₁₋₆)alkyl-O—(C₁₋₆)alkyl, —O-aryl-C(═O)OH, —C(═O)—O—(C₁₋₆)alkyl, —C(═O)NH₂, —C(═O)—N(H)—(C₁₋₆)alkyl-Het, —C(═O)—N(H)-Het and Het;
R¹ is independently selected from (C₁₋₆)alkyl, (C₁₋₆)haloalkyl, halo and (C₃₋₇)cycloalkyl;
R² is H, (C₁₋₆)alkyl, —(C₁₋₆)alkyl-(C₃₋₇)cycloalkyl or (C₃₋₇)cycloalkyl;
R³ is phenyl or thiophene, wherein said phenyl is optionally substituted in the meta- or para-position with (C₁₋₆)alkyl, halo, CN, OH or —O—(C₁₋₆)alkyl;
R⁴ is (C₁₋₆)alkyl optionally substituted 1 or 2 times with —O—(C₁₋₆)alkyl, Het, —COOH or OH;
R⁵ and R⁷ are each independently selected from OH, CN, halo, —COOH, R⁵¹, —O—R⁵¹, —S—R⁵¹, —SO—R⁵¹, —SO₂—R⁵¹, —C(═O)—NH₂, —C(═O)—N(R⁵¹)(R⁵²), —(C₁₋₆)alkyl-NH(R⁵¹), —(C₁₋₆)alkyl-O—R⁵¹ and —(C₁₋₆)alkyl-S—R⁵¹;
   R⁵¹ is selected from (C₁₋₆)alkyl, aryl, —(C₁₋₆)alkyl-aryl, Het and —(C₁₋₆)alkyl-Het;
   wherein said aryl and Het are optionally fused to ring D;
      wherein each said alkyl is optionally substituted 1 or 2 times with substituents independently selected from R⁵³;
      wherein each said aryl and Het are optionally substituted 1 to 2 times with substituents independently selected from R⁵³, —O—(C₁₋₆)alkyl, —OH, oxo, —C(═O)O(C₁₋₆)alkyl, —C(═O)—Het and (C₁₋₆)alkyl optionally substituted one time with Het or R⁵³;
      R⁵³ is —COOH, —NH₂, —NH(C₁₋₆)alkyl, —N((C₁₋₆)alkyl)₂, —O—(C₁₋₆)alkyl or —OH;
   R⁵² is selected from H and (C₁₋₆)alkyl;
Z¹ and Z² are either both defined as CH₂ and ---- is a single bond or Z¹ and Z² are both defined as CH and ---- is a double bond;
n, o and p are each independently selected from 0, 1, 2 or 3;
R⁶ is selected from (C₁₋₆)alkyl, —O—(C₁₋₆)alkyl, OH, NH₂, —N(H)(C₁₋₆)alkyl, —N((C₁₋₆)alkyl)₂, —N(H)—(C₁₋₆)alkyl-aryl, —N(H)—(C₁₋₆)alkyl-Het, —N(H)—C(═O)-aryl, —N(H)—C(═O)—Het, —N(H)—C(═O)—NH₂, oxo, Het and aryl,
   wherein each said aryl and Het are optionally substituted 1 to 2 times with substituents independently selected from R⁶¹, —O—(C₁₋₆)alkyl, —OH, —C(═O)—Het and (C₁₋₆)alkyl optionally substituted one time with R⁶¹;
   R⁶¹ is —COOH, —N((C₁₋₆)alkyl)₂ or —OH;
      or a salt thereof;
      with the proviso that the following compounds are excluded:
8-chloro-3-(methylamino-methylene)-1-phenyl-1,5-dihydro-benzo[b][1,4]diazepine-2,4-dione;
3-(butylamino-methylene)-8-chloro-1-phenyl-1,5-dihydro-benzo[b][1,4]diazepine-2,4-dione;
3-(tert-butylamino-methylene)-8-chloro-1-phenyl-1,5-dihydro-benzo[b][1,4]diazepine-2,4-dione;
8-chloro-3-(isobutylamino-methylene)-1-phenyl-1,5-dihydro-benzo[b][1,4]diazepine-2,4-dione;
8-bromo-3-(butylamino-methylene)-1-phenyl-1,5-dihydro-benzo[b][1,4]diazepine-2,4-dione; and
3-(butylamino-methylene)-1-phenyl-8-trifluoromethyl-1,5-dihydro-benzo[b][1,4]diazepine-2,4-dione.

Another aspect of this invention provides a compound of formula (I) or a pharmaceutically acceptable salt thereof, as a medicament.

Included within the scope of this invention is a pharmaceutical composition comprising a therapeutically effective amount of a compound of formula (I), or a pharmaceutically acceptable salt thereof, in admixture with at least one pharmaceutically acceptable carrier medium or auxiliary agent.

According to a further aspect of this invention, the pharmaceutical composition according to this invention further comprises a therapeutically effective amount of at least one other antiviral agent.

The invention also provides the use of a pharmaceutical composition as described hereinabove for the treatment of an HIV infection in a human being having or at risk of having the infection.

Another important aspect of the invention involves a method of treating or preventing an HIV infection in a human being by administering to the human being a therapeutically effective amount of a compound of formula (I), a pharmaceutically acceptable salt thereof, or a composition as described above, alone or in combination with at least one other antiviral agent, administered together or separately.

Also within the scope of this invention is the use of a compound of formula (I), or a pharmaceutically acceptable salt thereof, as described herein, for the manufacture of a medicament for the treatment or prevention of an HIV infection in a human being.

An additional aspect of this invention refers to an article of manufacture comprising a composition effective to treat an HIV infection; and packaging material comprising a label which indicates that the composition can be used to treat infection by HIV; wherein the composition comprises a compound of formula (I) according to this invention or a pharmaceutically acceptable salt thereof.

Still another aspect of this invention relates to a method of inhibiting the replication of HIV comprising exposing the virus to an effective amount of the compound of formula (I), or a salt thereof, under conditions where replication of HIV is inhibited.

Further included in the scope of the invention is the use of a compound of formula (I), or a salt thereof, to inhibit the replication of HIV.

### DETAILED DESCRIPTION OF THE INVENTION

### Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to. In the groups, radicals, or moieties defined below, the number of carbon atoms is often specified preceding the group, for example, C₁₋₆-alkyl means an alkyl group or radical having 1 to 6 carbon atoms. In general, for groups comprising two or more subgroups, the first named subgroup is the radical attachment point, for example, the substituent “—C₁₋₃-alkyl-aryl” means an aryl group which is bound to a —C₁₋₃-alkyl group, wherein the —C₁₋₃-alkyl-group is bound to the core. In the previous example of “—C₁₋₃-alkyl-aryl”, substituents may be attached to either the C₁₋₃-alkyl or aryl portion thereof or both, unless specified otherwise.

The term “C₁₋ₙ-alkyl”, wherein n is an integer from 2 to n, either alone or in combination with another radical denotes an acyclic, saturated, branched or linear hydrocarbon radical with 1 to n C atoms. For example the term C₁₋₅-alkyl includes, but is not limited to, the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH(CH₃)—, H₃C—CH(CH₃)—CH₂—CH₂—, H₃C—CH₂—C(CH₃)₂—, H₃C—C(CH₃)₂—CH₂—, and H₃C—CH₂—CH(CH₂CH₃)—.

The term “C₂₋ₙ-alkenyl”, is used for a group as defined in the definition for “C₁₋ₙ-alkyl” with at least two carbon atoms, if at least two of those carbon atoms of said group are bonded to each other by a double bond.

The term “C₂₋ₙ-alkynyl”, is used for a group as defined in the definition for “C₁₋ₙ-alkyl” with at least two carbon atoms, if at least two of those carbon atoms of said group are bonded to each other by a triple bond.

The term “carbocycle” means a mono- or multi-ring ring structure consisting only of carbon containing between one and four rings wherein such rings may be attached together in a pendent manner or may be fused. The term “carbocycle” refers to fully saturated and aromatic ring systems and partially saturated ring systems. The term “carbocycle” additionally encompasses spiro systems, and bridged systems.

The term “C₃₋ₙ-cycloalkyl”, wherein n is an integer 4 to n, either alone or in combination with another radical denotes a cyclic, saturated, unbranched hydrocarbon radical with 3 to n C atoms. For example the term C₃₋₇-cycloalkyl includes cyclopropyl, cyclobutyl, cyclopentyl, cyclohexyl and cycloheptyl.

The term “halo” generally denotes fluorine, chlorine, bromine and iodine.

The term “aryl” as used herein, either alone or in combination with another radical, denotes a carbocyclic aromatic monocyclic group containing 6 carbon atoms which may be further fused to a second 5- or 6-membered carbocyclic group which may be aromatic, saturated or unsaturated. Aryl includes, but is not limited to, phenyl, indanyl, indenyl, naphthyl, anthracenyl, phenanthrenyl, tetrahydronaphthyl and dihydronaphthyl.

The term “Het” as used herein, either alone or in combination with another radical, denotes a heterocyclyl or heteroaryl ring system.

The term “heterocyclyl” means a saturated or unsaturated mono- or polycyclic-ring systems including aromatic ring system containing one or more heteroatoms selected from N, O or S(O)ᵣ with r=0, 1 or 2 wherein none of the heteroatoms is part of the aromatic ring. The term “heterocyclyl” is intended to include all the possible isomeric forms. Thus, the term “heterocyclyl” includes the following exemplary structures which are not depicted as radicals as each form may be attached through a covalent bond to any atom so long as appropriate valences are maintained:

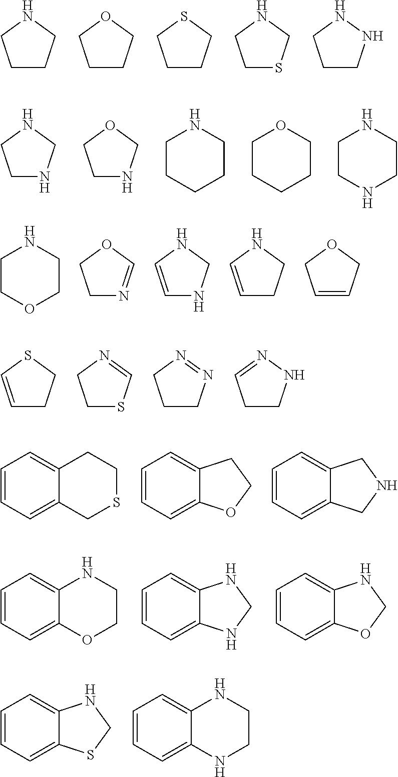

The term “heteroaryl” means a mono- or polycyclic-ring systems containing one or more heteroatoms selected from N, O or S(O)ᵣ with r=0, 1 or 2 wherein at least one of the heteroatoms is part of aromatic ring. The term “heteroaryl” is intended to include all the possible isomeric forms. Thus, the term “heteroaryl” includes the following exemplary structures which are not depicted as radicals as each form may be attached through a covalent bond to any atom so long as appropriate valences are maintained:

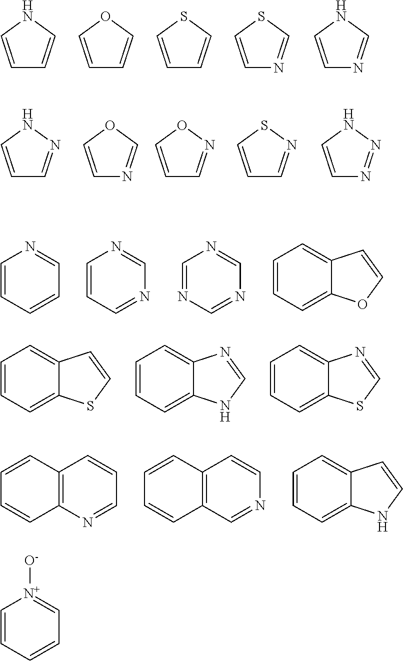

Many of the terms given above may be used repeatedly in the definition of a formula or group and in each case have one of the meanings given above, independently of one another.

In case a compound of the present invention or an intermediate used in the synthesis of a compound of the present invention is depicted in the form of a chemical name and as a formula, the formula shall prevail in case of any discrepancy between the name and formula.

An asterisk or the designation

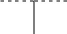

is used in sub-formulas to indicate the bond which is connected to the core molecule as defined.

As used herein, the designation whereby a bond to a substituent R is drawn as emanating from the center of a ring system, such as, for example,

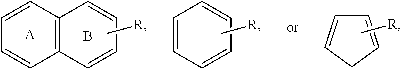

is intended to mean that the substituent R may be attached to any free position on the ring system that would otherwise be substituted with a hydrogen atom, unless specified otherwise. In a polycyclic ring system, such as the naphthalene example shown above, the substituent R may be attached to any free position on ring A or B, unless specified otherwise.

Unless specifically indicated, throughout the specification and the appended claims, a given chemical formula or name shall encompass tautomers and all stereo, optical and geometrical isomers (e.g. enantiomers, diastereomers, E/Z isomers, atropisomers) and racemates thereof as well as mixtures in different proportions of the separate enantiomers, mixtures of diastereomers, or mixtures of any of the foregoing forms where such isomers and enantiomers exist, as well as salts, including pharmaceutically acceptable salts thereof and solvates thereof such as for instance hydrates including solvates of the free compounds or solvates of a salt of the compound.

One skilled in the art would know how to separate, enrich, or selectively prepare the enantiomers of the compounds of the present invention. Preparation of pure stereoisomers, e.g. enantiomers and diastereomers, or mixtures of desired enantiomeric excess (ee) or enantiomeric purity, are accomplished by one or more of the many methods of (a) separation or resolution of enantiomers, or (b) enantioselective synthesis known to those of skill in the art, or a combination thereof. These resolution methods generally rely on chiral recognition and include but not limited to chromatography using chiral stationary phases, enantioselective host-guest complexation, resolution or synthesis using chiral auxiliaries, enantioselective synthesis, enzymatic and nonenzymatic kinetic resolution, or spontaneous enantioselective crystallization. Such methods are disclosed generally in Chiral Separation Techniques: A Practical Approach (2nd Ed.), G. Subramanian (ed.), Wiley-VCH, 2000; T. E. Beesley and R. P. W. Scott, Chiral Chromatography, John Wiley & Sons, 1999; and Satinder Ahuja, Chiral Separations by Chromatography, Am. Chem. Soc., 2000. Furthermore, there are equally well-known methods for the quantitation of enantiomeric excess or purity, including but not limited to GC, HPLC, CE, or NMR, and assignment of absolute configuration and conformation, including but not limited to CD, ORD, X-ray crystallography, or NMR.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, and commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. For example, such salts include acetates, ascorbates, benzenesulfonates, benzoates, besylates, bicarbonates, bitartrates, bromides/hydrobromides, Ca-edetates/edetates, camsylates, carbonates, chlorides/hydrochlorides, citrates, edisylates, ethane disulfonates, estolates esylates, fumarates, gluceptates, gluconates, glutamates, glycolates, glycollylarsnilates, hexylresorcinates, hydrabamines, hydroxymaleates, hydroxynaphthoates, iodides, isothionates, lactates, lactobionates, malates, maleates, mandelates, methanesulfonates, mesylates, methylbromides, methylnitrates, methylsulfates, mucates, napsylates, nitrates, oxalates, pamoates, pantothenates, phenylacetates, phosphates/diphosphates, polygalacturonates, propionates, salicylates, stearates subacetates, succinates, sulfamides, sulfates, tannates, tartrates, teoclates, toluenesulfonates, triethiodides, ammonium, benzathines, chloroprocaines, cholines, diethanolamines, ethylenediamines, meglumines and procaines. Further pharmaceutically acceptable salts can be formed with cations from metals like aluminium, calcium, lithium, magnesium, potassium, sodium, zinc and the like. (also see Pharmaceutical salts, Berge, S. M. et al., J. Pharm. Sci., (1977), 66, 1-19; and Handbook of Pharmaceutical Salts, P. Heinrich Stahl, Camille G. Wermuth (Eds.), Wiley-VCH, 2002).

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent like ether, ethyl acetate, ethanol, isopropanol, or acetonitrile, or a mixture thereof.

Salts of other acids than those mentioned above which for example are useful for purifying or isolating the compounds of the present invention (e.g. trifluoro acetate salts) also comprise a part of the invention.

The term “treatment” as used herein is intended to mean the administration of a compound or composition according to the present invention to alleviate or eliminate symptoms of HIV infection and/or to reduce viral load in a patient. The term “treatment” also encompasses the administration of a compound or composition according to the present invention post-exposure of the individual to the virus but before the appearance of symptoms of the disease, and/or prior to the detection of the virus in the blood, to prevent the appearance of symptoms of the disease and/or to prevent the virus from reaching detectable levels in the blood, and the administration of a compound or composition according to the present invention to prevent perinatal transmission of HIV-1 from mother to baby, by administration to the mother before giving birth and to the child within the first days of life.

The term “antiviral agent” as used herein is intended to mean an agent that is effective to inhibit the formation and/or replication of a virus in a mammal, including but not limited to agents that interfere with either host or viral mechanisms necessary for the formation and/or replication of a virus in a mammal.

### PREFERRED EMBODIMENTS

In the following preferred embodiments, groups and substituents of the compounds of Formula (I) according to this invention are described in detail.

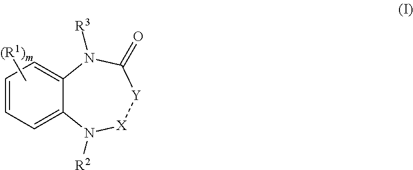

Any and each of the following definitions may be combined with each other.

### X/Y:

X/Y-a: X and Y are

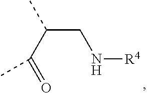

and Formula (I) has the structure:

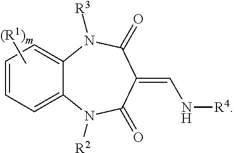

X/Y-b: X and Y are

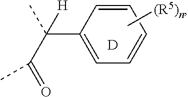

and Formula (I) has the structure:

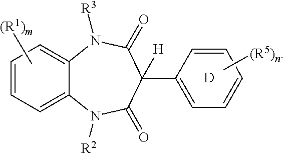

X/Y-c: X and Y are

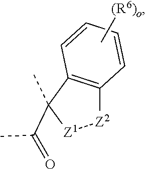

and Formula (I) has the structure:

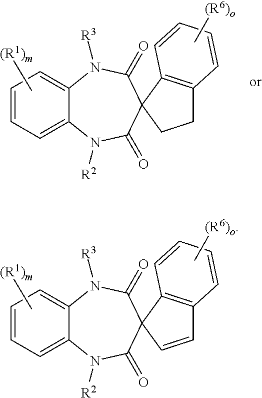

X/Y-d: X and Y are

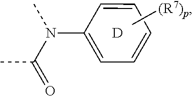

and Formula (I) has the structure:

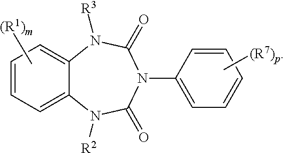

X/Y-e: X and Y are linked to form a 5-membered heteroaryl ring containing 1 to 3 heteroatoms independently selected from O, N and S, and Formula (I) has the structure

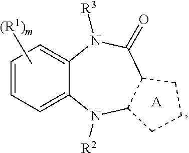

wherein the 5-membered heteroaryl ring A is optionally substituted 1 to 2 times with substituents independently selected from (C₁₋₆)alkyl, (C₂₋₆)alkenyl, (C₂₋₆)alkynyl, (C₃₋₇)cycloalkyl, —(C₁₋₆)alkyl-(C₃₋₇)cycloalkyl, —(C₁₋₆)alkyl-aryl, aryl, —C(═O)—(C₁₋₆)alkyl, —C(═O)—NH₂, —C(═O)—N((C₁₋₆)alkyl)₂ and —SO₂—(C₁₋₆)alkyl;
   wherein each said alkyl, aryl and Het, either alone or in combination with another radical is optionally substituted 1 to 2 times with substituents independently selected from (C₁₋₆)alkyl, OH, CN, —COOH, halo, —(C₁₋₆)alkyl-Het, —(C₁₋₆)alkyl-aryl, —NH₂, —NH(C₁₋₆)alkyl, —N((C₁₋₆)alkyl)₂, —N(H)—C(═O)—O—(C₁₋₆)alkyl, N(H)—(C₁₋₆)alkyl-O—(C₁₋₆)alkyl, —O-aryl-C(═O)OH, —C(═O)—O—(C₁₋₆)alkyl, —C(═O)NH₂, —C(═O)—N(H)—(C₁₋₆)alkyl-Het, —C(═O)—N(H)-Het and Het.
      m:
m-A: m is 1, 2 or 3.
m-B: m is 1 or 2.
m-C: m is 1.

### R¹:

R¹-A: R¹ is independently selected from (C₁₋₆)alkyl, (C₁₋₆)haloalkyl, halo and (C₃₋₇)cycloalkyl.
R¹-B: R¹ is independently selected from (C₁₋₃)alkyl, (C₁₋₃)haloalkyl, F, Cl and (C₃₋₅)cycloalkyl.
R¹-C: R¹ is independently selected from Cl, F, CH₃ and CF₃.
R¹-D: R¹ is independently selected from Cl and CF₃.
R¹-E: R¹ is CF₃.

### R²:

R²-A: R² is H, —(C₁₋₆)alkyl-(C₃₋₇)cycloalkyl or (C₃₋₇)cycloalkyl.
R²-B: R² is H or (C₁₋₆)alkyl.
R²-C: R² is H or (C₁₋₃)alkyl.
R²-D: R² is H, CH₃ or CH₂CH₃.
R²-E: R² is CH₃ or CH₂CH₃.

### R³:

R³-A: R³ is phenyl or thiophene, wherein said phenyl is optionally substituted in the meta- or para-position with (C₁₋₆)alkyl, halo, CN, OH or —O—(C₁₋₆)alkyl.
R³-B: R³ is phenyl or thiophene.
R³-C: R³ is phenyl.
R³-D: R³ is thiophene.

### R⁴:

R⁴-A: R⁴ is (C₁₋₆)alkyl optionally substituted 1 or 2 times with —O—(C₁₋₆)alkyl, Het, COOH or OH.
R⁴-B: R⁴ is (C₁₋₄)alkyl optionally substituted one time with —O—(C₁₋₄)alkyl, 5-membered Het, COOH or OH.
R⁴-C: R⁴ is (C₁₋₄)alkyl optionally substituted one time with —O—(C₁₋₃)alkyl or 5-membered Het; and
   Het is defined as a saturated or unsaturated monocyclic ring system including aromatic ring system containing one heteroatom selected from N, O or S.

### R⁵:

R⁵-A: R⁵ is selected from OH, CN, halo, —COOH, R⁵¹, —O—R⁵¹, —S—R⁵¹, —SO—R⁵¹, —SO₂—R⁵¹, —C(═O)—NH₂, —C(═O)—N(R⁵¹)(R⁵²), —(C₁₋₆)alkyl-NH(R⁵¹), O—R⁵¹ and —(C₁₋₆)alkyl-S—R⁵¹;
   R⁵¹ is selected from (C₁₋₆)alkyl, aryl, —(C₁₋₆)alkyl-aryl, Het and —(C₁₋₆)alkyl-Het;
   wherein said aryl and Het are optionally fused to ring D;
      wherein each said alkyl is optionally substituted 1 or 2 times with substituents independently selected from R⁵³;
      wherein each said aryl and Het are optionally substituted 1 to 2 times with substituents independently selected from R⁵³, —OH, oxo, —C(═O)O(C₁₋₆)alkyl, —C(═O)—Het and (C₁₋₆)alkyl optionally substituted one time with Het or R⁵³;
         R⁵³ is —COOH, —NH₂, —NH(C₁₋₆)alkyl, —N((C₁₋₆)alkyl)₂, —O—(C₁₋₆)alkyl or —OH;
   R⁵² is selected from H and (C₁₋₆)alkyl.
R⁵-B: R⁵ is selected from OH, CN, halo, —COOH, R⁵¹, —O—R⁵¹, —S—R⁵¹, —SO—R⁵¹, —SO₂—R⁵¹ and —C(═O)—NH₂;
   R⁵¹ is selected from (C₁₋₃)alkyl, Het and —(C₁₋₃)alkyl-Het;
   wherein said Het is optionally fused to ring D;
      wherein each said alkyl is optionally substituted 1 or 2 times with substituents independently selected from R⁵³;
      wherein each said phenyl and Het are optionally substituted 1 to 2 times with substituents independently selected from R⁵³, —O—(C₁₋₃)alkyl, —OH, oxo and (C₁₋₃)alkyl optionally substituted one time with Het or R⁵³;
      R⁵³ is —COOH, —N((C₁₋₃)alkyl)₂, or —OH; and
   Het is defined as a saturated or unsaturated 5- or 6-membered monocyclic ring system including aromatic ring system containing one heteroatom selected from N, O or S.
      n:
n-A: n is 0, 1, 2 or 3.
n-B: n is 0, 1 or 2.
n-C: n is 0 or 1.

### R⁶:

R⁶-A: R⁶ is selected from (C₁₋₆)alkyl, —O—(C₁₋₆)alkyl, OH, NH₂, —N(H)(C₁₋₆)alkyl, —N((C₁₋₆)alkyl)₂, —N(H)—(C₁₋₆)alkyl-aryl, N(H)—(C₁₋₆)alkyl-Het, —N(H)—C(═O)-aryl, —N(H)—C(═O)—Het, —N(H)—C(═O)—NH₂, oxo, Het and aryl,
   wherein each said aryl and Het are optionally substituted 1 to 2 times with substituents independently selected from R⁶¹, —O—(C₁₋₆)alkyl, —OH, —C(═O)—Het and (C₁₋₆)alkyl optionally substituted one time with R⁶¹;
      R⁶¹ is —COOH, —N((C₁₋₆)alkyl)₂ or —OH.
R⁶-B: R⁶ is selected from (C₁₋₃)alkyl, —O—(C₁₋₃)alkyl, OH, NH₂, —N(H)(C₁₋₆)alkyl, —N((C₁₋₆)alkyl)₂, —N(H)—C(═O)-phenyl, —N(H)—C(═O)—Het, oxo, Het and phenyl,
   wherein each said phenyl and Het are optionally substituted 1 to 2 times with substituents independently selected from R⁶¹, —O—(C₁₋₃)alkyl, —OH, —C(═O)—Het and (C₁₋₃)alkyl optionally substituted one time with R⁶¹;
   R⁶¹ is —COOH, —N((C₁₋₃)alkyl)₂ or —OH; and
   Het is defined as a saturated or unsaturated 5- or 6-membered monocyclic ring system including aromatic ring system containing 1 to 3 heteroatoms selected from N, O or S.
      o:
o-A: o is 0, 1, 2 or 3.
o-B: o is 0, 1 or 2.
o-C: o is 0 or 1.

### R⁷:

R⁷-A: R⁷ is selected from OH, CN, halo, —COOH, R⁵¹, —O—R⁵¹, —S—R⁵¹, —SO—R⁵¹, —SO₂—R⁵¹, —C(═O)—NH₂, —C(═O)—N(R⁵¹)(R⁵²), —(C₁₋₆)alkyl-NH(R⁵¹), O—R⁵¹ and —(C₁₋₆)alkyl-S—R⁵¹;
   R⁵¹ is selected from (C₁₋₆)alkyl, aryl, —(C₁₋₆)alkyl-aryl, Het and —(C₁₋₆)alkyl-Het;
   wherein said aryl and Het are optionally fused to ring D;
      wherein each said alkyl is optionally substituted 1 or 2 times with substituents independently selected from R⁵³;
      wherein each said aryl and Het are optionally substituted 1 to 2 times with substituents independently selected from R⁵³, —O—(C₁₋₆)alkyl, —OH, oxo, —C(═O)O(C₁₋₆)alkyl, —C(═O)—Het and (C₁₋₆)alkyl optionally substituted one time with Het or R⁵³;
         R⁵³ is —COOH, —NH₂, —NH(C₁₋₆)alkyl, —N((C₁₋₆)alkyl)₂ or —OH;
   R⁵² is selected from H and (C₁₋₆)alkyl.
R⁷-B: R⁷ is selected from OH, halo, —COOH, R⁵¹, —O—R⁵¹, C(═O)—NH(R⁵¹), —(C₁₋₃)alkyl-NH(R⁵¹), —(C₁₋₃)alkyl-O—R⁵¹ and —(C₁₋₃)alkyl-S—R⁵¹;
   R⁵¹ is selected from (C₁₋₃)alkyl, phenyl, —(C₁₋₃)alkyl-phenyl, Het and —(C₁₋₃)alkyl-Het;
      wherein each said alkyl is optionally substituted 1 or 2 times with substituents independently selected from R⁵³;
      wherein each said phenyl and Het are optionally substituted 1 to 2 times with substituents independently selected from R⁵³, —O—(C₁₋₃)alkyl, —OH and (C₁₋₃)alkyl optionally substituted one time with Het or R⁵³;
         R⁵³ is —COOH, —NH₂, —NH(C₁₋₃)alkyl, —N((C₁₋₃)alkyl)₂ or —OH; and
      Het is defined as a saturated or unsaturated 5- or 6-membered monocyclic ring system including aromatic ring system containing 1 to 3 heteroatoms selected from N.
         p:
p-A: p is 0, 1, 2 or 3.
p-B: p is 0, 1 or 2.
p-C: p is 0 or 1.

### Ring A:

A-A: Ring A is a 5-membered heteroaryl ring containing 1 or 2 heteroatoms independently selected from O, N and S, wherein said 5-membered heteroaryl ring is optionally substituted 1 to 2 times with substituents independently selected from (C₁₋₆)alkyl, (C₂₋₆)alkenyl, (C₂₋₆)alkynyl, (C₃₋₇)cycloalkyl, —(C₁₋₆)alkyl-(C₃₋₇)cycloalkyl, —(C₁₋₆)alkyl-aryl, aryl, —C(═O)—(C₁₋₆)alkyl, —C(═O)—NH₂, —C(═O)—N((C₁₋₆)alkyl)₂ and —SO₂—(C₁₋₆)alkyl;
   wherein each said alkyl, aryl and Het, either alone or in combination with another radical is optionally substituted 1 to 2 times with substituents independently selected from (C₁₋₆)alkyl, OH, CN, COOH, halo, —(C₁₋₆)alkyl-Het, —(C₁₋₆)alkyl-aryl, —NH₂, —NH(C₁₋₆)alkyl, —N((C₁₋₆)alkyl)₂, —N(H)—C(═O)—O—(C₁₋₆)alkyl, —(C₁₋₆)alkyl-N(H)—(C₁₋₆)alkyl-O—(C₁₋₆)alkyl, —O-aryl-C(═O)OH, —C(═O)—O—(C₁₋₆)alkyl, —C(═O)NH₂, —C(═O)—N(H)—(C₁₋₆)alkyl-Het, —C(═O)—N(H)-Het and Het.
A-B: Ring A is a 5-membered heteroaryl ring selected from:

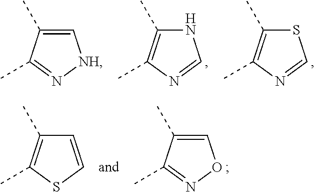

wherein said 5-membered heteroaryl ring is optionally substituted 1 to 2 times with substituents independently selected from (C₁₋₆)alkyl, (C₂₋₄)alkenyl, (C₂₋₄)alkynyl, (C₃₋₅)cycloalkyl, —(C₁₋₃)alkyl-(C₃₋₅)cycloalkyl, —(C₁₋₃)alkyl-phenyl, phenyl, —C(═O)—(C₁₋₃)alkyl, —C(═O)—NH₂, —C(═O)—N((C₁₋₃)alkyl)₂ and —SO₂—(C₁₋₃)alkyl;
   wherein each said alkyl, phenyl and Het, either alone or in combination with another radical is optionally substituted 1 to 2 times with substituents independently selected from (C₁₋₃)alkyl, —O—(C₁₋₃)alkyl, OH, CN, COOH, halo, —(C₁₋₃)alkyl-Het, —NH₂, —N((C₁₋₃)alkyl)₂, —N(H)—C(═O)—O—(C₁₋₃)alkyl, —(C₁₋₃)alkyl-N(H)—(C₁₋₆)alkyl-O—(C₁₋₃)alkyl, —O-phenyl-C(═O)OH, —C(═O)—O—(C₁₋₃)alkyl —C(═O)NH₂, —C(═O)—N(H)—(C₁₋₃)alkyl-Het and —C(═O)—N(H)-Het; and
   Het is defined as an unsaturated 5- or 6-membered monocyclic ring system including aromatic ring system containing one or more heteroatoms selected from N or O.

The following table represents further embodiments E1 to E15 of the compounds of Formula (I):

When X/Y is X/Y-a, the embodiments are:

| Embodiment | m | R¹ | R² | R³ | R⁴ |
|---|---|---|---|---|---|
| E1 | m-B | R¹-A | R²-D | R³-B | R⁴-C |
| E2 | m-C | R¹-C | R²-B | R³-B | R⁴-B |
| E3 | m-C | R¹-C | R²-C | R³-C | R⁴-B |

When X/Y is X/Y-b, the embodiments are:

| Embodiment | m | R¹ | R² | R³ | R⁵ | n |
|---|---|---|---|---|---|---|
| E4 | m-C | R¹-D | R²-B | R³-B | R⁵-B | n-A |
| E5 | m-B | R¹-B | R²-E | R³-C | R⁵-A | n-B |
| E6 | m-C | R¹-B | R²-B | R³-B | R⁵-B | n-C |

When X/Y is X/Y-c, the embodiments are:

| Embodiment | m | R¹ | R² | R³ | R⁶ | o |
|---|---|---|---|---|---|---|
| E7 | m-B | R¹-B | R²-B | R³-B | R⁶-B | o-B |
| E8 | m-C | R¹-D | R²-B | R³-C | R⁶-A | o-C |
| E9 | m-C | R¹-B | R²-E | R³-C | R⁶-B | o-C |

When X/Y is X/Y-d, the embodiments are:

| Embodiment | m | R¹ | R² | R³ | R⁷ | p |
|---|---|---|---|---|---|---|
| E10 | m-B | R¹-B | R²-B | R³-B | R⁷-B | p-B |
| E11 | m-C | R¹-D | R²-B | R³-C | R⁷-C | p-C |
| E12 | m-C | R¹-E | R²-E | R³-C | R⁷-B | p-C |

When X/Y is X/Y-e, the embodiments are:

| Embodiment | m | R¹ | R² | R³ | Ring A |
|---|---|---|---|---|---|
| E13 | m-B | R¹-C | R²-E | R³-B | A-A |
| E14 | m-C | R¹-D | R²-B | R³-C | A-B |
| E15 | m-C | R¹-B | R²-E | R³-C | A-B |

Examples of the most preferred compounds according to this invention are each single compound listed in Tables 1 to 7.

### Pharmaceutical Composition

Suitable preparations for administering the compounds of Formula I will be apparent to those with ordinary skill in the art and include for example tablets, pills, capsules, suppositories, lozenges, troches, solutions, syrups, elixirs, sachets, injectables, inhalatives and powders etc. The content of the pharmaceutically active compound(s) should be in the range from 0.05 to 90 wt.-%, preferably 0.1 to 50 wt.-% of the composition as a whole.

Suitable tablets may be obtained, for example, by mixing one or more compounds according to formula I with known excipients, for example inert diluents, carriers, disintegrants, adjuvants, surfactants, binders and/or lubricants. The tablets may also consist of several layers.

The dose range of the compounds of the invention applicable per day is usually from 0.01 to 100 mg/kg of body weight, preferably from 0.1 to 50 mg/kg of body weight. Each dosage unit may conveniently contain from 5% to 95% active compound (w/w). Preferably such preparations contain from 20% to 80% active compound.

The actual pharmaceutically effective amount or therapeutic dosage will of course depend on factors known by those skilled in the art such as age and weight of the patient, route of administration and severity of disease. In any case the combination will be administered at dosages and in a manner which allows a pharmaceutically effective amount to be delivered based upon patient's unique condition.

### Combination Therapy

When the composition of this invention comprises a combination of a compound of the invention and one or more additional therapeutic or prophylactic agent, both the compound and the additional agent should be present at dosage levels of between about 10 to 100%, and more preferably between about 10 and 80% of the dosage normally administered in a monotherapy regimen. Therefore, according to one embodiment, the pharmaceutical composition of this invention additionally comprises one or more antiviral agents.

Antiviral agents contemplated for use in such combination therapy include agents (compounds or biologicals) that are effective to inhibit the formation and/or replication of a virus in a mammal, including but not limited to agents that interfere with either host or viral mechanisms necessary for the formation and/or replication of a virus in a mammal. Such agents can be selected from:
NRTIs (nucleoside or nucleotide reverse transcriptase inhibitors; including but not limited to zidovudine, didanosine, zalcitabine, stavudine, lamivudine, emtricitabine, abacavir, tenofovir, festinavir (OBP-601), elvucitabine, apricitabine);
NNRTIs (non-nucleoside reverse transcriptase inhibitors; including but not limited to nevirapine, delavirdine, efavirenz, etravirine, rilpivirine, BILR 355, RDEA806, lersivirine (UK435061) and GSK2248761 (IDX-899));
protease inhibitors (including but not limited to ritonavir, tipranavir, saquinavir, nelfinavir, indinavir, amprenavir, fosamprenavir, atazanavir, lopinavir, darunavir, brecanavir, TMC-310911, PPL-100 (MK-8122), DG17 and SPI-256);
entry inhibitors including but not limited to
   CCR5 antagonists (including but not limited to maraviroc (UK-427,857), vicriviroc (SCH-D, SCH-417690), TAK-652, INCB9471, PF-232798, PRO-140, TBR-652, SCH532706 and TBR-220),
   CXCR4 antagonists (including but not limited to AMD-11070),
   fusion inhibitors (including but not limited to enfuvirtide (T-20), sifuvirtide, albuvirtide and TRI-1144) and
   others (including but not limited to ibalizumab (TNX-355) and BMS-488043);
integrase inhibitors (including but not limited to raltegravir (MK-0518), c-1605, BMS-538158, elvitegravir (GS 9137), GSK1349572, GSK 1265744 and JTK-656);
TAT inhibitors;
maturation inhibitors (including but not limited to bevirimat (PA-457)); and
immunomodulating agents (including but not limited to levamisole).

Furthermore, a compound according to the invention can be used with at least one other compound according to the invention or with one or more antifungal or antibacterial agents (including but not limited to fluconazole).

### EXAMPLES

Other features and advantages of the present invention will become apparent from the following more detailed Examples which illustrate, by way of example, the principles of the invention. As is well known to a person skilled in the art, reactions are performed in an inert atmosphere (including but not limited to nitrogen or argon) where necessary to protect reaction components from air or moisture. Solution percentages and ratios express a volume to volume relationship, unless stated otherwise. Flash chromatography is carried out on silica gel (SiO₂) according to the procedure of W. C. Still et al., J. Org. Chem., (1978), 43, 2923. Mass spectral analyses are recorded using electrospray mass spectrometry.

Preparative HPLC is carried out using a Combiprep ODS-AQ column, 50×20 mm, 5 μm, 120 Å, elution with a gradient of CH₃CN/H₂O containing 0.06% TFA.

Analytical UPLC (Method A) is carried out under standard conditions using an Aquity™ HSST3 (1.8 μm, 2.1×50 mm) reverse phase column at 220 nm, elution with a linear gradient as described in the following table (Solvent A 10 mM Ammonium Formate in H₂O (pH=3.8); solvent B MeOH):

| Time (min) | Flow (mL/min) | Solvent A (%) | Solvent B (%) |
|---|---|---|---|
| 0 | 0.8 | 95 | 5 |
| 2.30 | 0.9 | 0 | 100 |
| 2.32 | 1.0 | 0 | 100 |
| 3.00 | 1.0 | 0 | 100 |

Alternatively, analytical HPLC (Method B) is carried out under standard conditions using a Combiscreen ODS-AQ C18 reverse phase column, YMC, 50×4.6 mm i.d., 5 μm, 120 Å at 220 nM, elution with a linear gradient as described in the following table (Solvent A is 0.06% TFA in H₂O; solvent B is 0.06% TFA in CH₃CN):

| Time (min) | Flow (mL/min) | Solvent A (%) | Solvent B (%) |
|---|---|---|---|
| 0 | 3.0 | 95 | 5 |
| 0.5 | 3.0 | 95 | 5 |
| 6.0 | 3.0 | 50 | 50 |
| 10.5 | 3.5 | 0 | 100 |

Alternatively, analytical HPLC (Method C) is carried out under standard conditions using a Combiscreen ODS-AQ C18 reverse phase column, YMC, 50×4.6 mm i.d., 5 μm, 120 Å at 220 nM, elution with a linear gradient as described in the following table (Solvent A is 0.06% TFA in CH₃CN; solvent B is 0.06% TFA in H₂O):

| Time (min) | Flow (mL/min) | Solvent A (%) | Solvent B (%) |
|---|---|---|---|
| 0 | 0.70 | 5 | 95 |
| 0.5 | 0.70 | 5 | 95 |
| 4.0 | 0.70 | 50 | 50 |
| 6.5 | 0.85 | 100 | 0 |
| 8.0 | 1.0 | 100 | 0 |
| 8.1 | 0.7 | 5 | 95 |
| 8.6 | 0.70 | 5 | 95 |

Purification using CombiFlash® Companion or RF apparatus (Teledyne Isco Inc) employs pre-packed silica gel cartridges and EtOAc and hexanes as solvents. These cartridges are available either from Silicycle Inc (SiliaFlash, 40-63 μm silica) or from Teledyne Isco (RediSep, 40-63 μm silica).

Abbreviations or symbols used herein include:

Ac: acetyl; AcOH: acetic acid; AIBN: azobisisobutyronitrile; Bn: benzyl (phenylmethyl); BOC or Boc: tert-butyloxycarbonyl; Bu: butyl; CAA: capsid assembly assay; CDA: capsid disassembly assay; c-Pr: cyclopropyl; DBU: 1,8-diazabicyclo[5.4.0]undec-7-ene; DCM: dichloromethane; DEAD: diethyl azodicarboxylate; DIAD: diisopropyl azodicarboxylate; DIPEA: diisopropylethylamine; DMF: N,N-dimethylformamide; DMSO: dimethylsulfoxide; EC₅₀: 50% effective concentration; Et: ethyl; Et₃N: triethylamine; Et₂O: diethyl ether; EtOAc: ethyl acetate; Hex: hexane; HATU: N,N,N′,N′-tetramethyl-O-(7-azabenzotriazol-1-yl)uronium hexafluorophosphate; HPLC: high performance liquid chromatography; IC₅₀: 50% inhibitory concentration; ⁱPr or i-Pr: 1-methylethyl (iso-propyl); LC-MS: liquid chromatography-mass spectrometry; HMDS: lithium hexamethyl disilazide; m/z: mass-to-charge ratio; [M+H]⁺: protonated molecular ion; m-CPBA: meta-chloroperbenzoic acid; Me: methyl; MeCN: acetonitrile; MeOH: methanol; MS: mass spectrometry; MsCl: methanesulfonyl chloride; NBS: N-bromosuccinimide; Ph: phenyl; PMB: p-methoxybenzyl; Pr: propyl; Prep LCMS: preparative liquid chromatography-mass spectrometry; p-TsOH: p-toluenesulfonic acid; RT: room temperature (approximately 18° C. to 25° C.); tert-butyl, t-butyl or t-Bu: 1,1-dimethylethyl; TBAF: tetrabutylammonium fluoride; TBDMS: ten-butyldimethylsilyl; TBTU: O-(Benzotriazol-1-yl)-N,N,N′,N′-tetramethyluronium tetrafluoroborate; TFA: trifluoroacetic acid; Tf: trifluoromethanesulfonyl; Tf₂O: trifluoromethanesulfonic anhydride; TfOH: trifluoromethanesulfonic acid; THF: tetrahydrofuran; TLC: thin layer chromatography; TMSOTf: trifluoromethanesulfonic acid trimethylsilylester; t_{R}: retention time; UPLC: ultra performance liquid chromatography.

### Example 1a

### Preparation of Compound 1a5

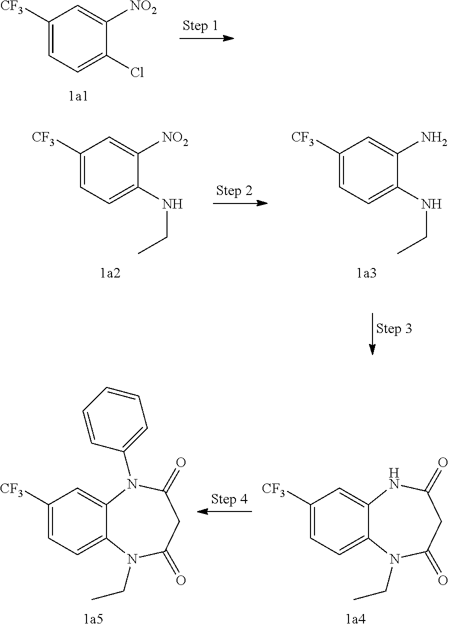 [SMILES: O=[N+]([O-])c1cc(C(F)(F)F)ccc1Cl ; CCNc1ccc(C(F)(F)F)cc1[N+](=O)[O-] ; CCNc1ccc(C(F)(F)F)cc1N ; CCN1C(=O)CC(=O)N(c2ccccc2)c2cc(C(F)(F)F)ccc21 ; CCN1C(=O)CC(=O)Nc2cc(C(F)(F)F)ccc21]

### Step 1:

Et₃N (92 mL, 660 mmol) is added to a solution of 1a1 (Aldrich; 34 mL, 220 mmol) and EtNH₂.HCl (20 g, 240 mmol) dissolved in DMF and the reaction mixture is stirred at 100° C. for 1 h. The reaction mixture is diluted with EtOAc, washed with water followed by brine, dried over MgSO₄, filtered and concentrated under vacuum to give compound 1a2.

### Step 2:

Formic acid (43 mL, 1.2 mol) is carefully added to a mixture of 1a2 (45 g, 190 mmol), Et₃N (170 mL, 1.2 mol) and 10% Pd/C (20 g) in EtOH (600 mL) under a N₂ atmosphere. The mixture is stirred for 2 h and then is filtered through Celite®. The filtrate is concentrated and the crude product is diluted with EtOAc, washed with saturated NaHCO₃, water, brine, dried (MgSO₄), filtered and concentrated under vacuum to give compound 1a3.

### Step 3:

A solution of 1a3 (14 g, 66 mmol) in THF (100 mL) and a solution of malonyl dichloride (8.4 mL, 86 mmol) in THF (100 mL) are added dropwise simultaneously with rapid stirring over 40 min to a flask charged with THF (50 mL) at RT under a N₂ atmosphere. The reaction is stirred for 30 min and concentrated under vacuum. The residue is dissolved in CHCl₃ (100 mL) and hexane (500 mL) is added. The mixture is filtered and the solid is collected and dried under vacuum to give compound 1a4.

### Step 4:

Ph₃Bi (48 g, 110 mmol), Cu(OAc)₂ (20 g, 110 mmol) and pyridine (8.8 mL, 110 mmol) are sequentially added to a solution of 1a4 in DCM (400 mL) at RT. The reaction mixture is stirred at 50° C. for 8 h, cooled to RT and filtered through silica gel, first eluting with DCM followed by 9:1 DCM/Acetone. The DCM/acetone fraction is concentrated and the residue is purified by flash chromatography (DCM:Acetone 9:1) to give compound 1a5.

### Example 1b

### Preparation of Compound 1003

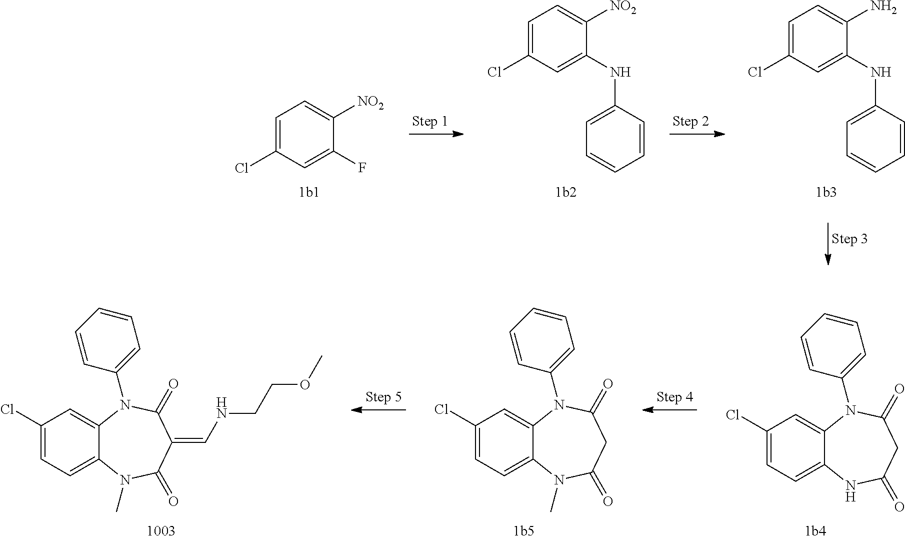 [SMILES: O=[N+]([O-])c1ccc(Cl)cc1F ; O=[N+]([O-])c1ccc(Cl)cc1Nc1ccccc1 ; Nc1ccc(Cl)cc1Nc1ccccc1 ; COCCN/C=C1/C(=O)N(C)c2ccc(Cl)cc2N(c2ccccc2)C1=O ; CN1C(=O)CC(=O)N(c2ccccc2)c2cc(Cl)ccc21 ; O=C1CC(=O)N(c2ccccc2)c2cc(Cl)ccc2N1]

### Step 1:

Et₃N (23.7 mL, 170 mmol) is added to a solution of 1b1 (TCI-US; 15 g, 90 mmol) and aniline (9.3 mL, 100 mmol) in DMSO (45 mL). The reaction mixture is stirred at RT for 43 h. The reaction mixture is diluted with water, stirred at 10° C. for 30 min and filtered. The resulting solid is washed with water and dried under vacuum to give compound 1b2.

### Step 2:

To 1b2 (18.5 g, 70 mmol) in EtOH (110 mL), water (37 mL) and aqueous 1N HCl solution (6.5 mL) is added iron powder (16.5 g, 300 mmol). The mixture is heated to reflux for 1 h before being cooled and filtered through Celite®. The filtrate is concentrated and the crude product is diluted in DCM/water before being neutralized with NaHCO₃. The organic phase is washed with brine and dried (MgSO₄), filtered and concentrated under vacuum. The crude material is purified by CombiFlash (DCM) to give 1b3.

### Step 3:

A solution of 1b3 (3.2 g, 14.5 mmol) in THF (75 mL) and a solution of malonyl dichloride (1.48 mL, 15.3 mmol) in THF (45 mL) are simultaneously added dropwise to a reaction flask charged with THF (50 mL) at RT with rapid stirring over 1 h. The reaction mixture is stirred for 30 min and then concentrated under vacuum. The residue is dissolved in CHCl₃ (65 mL) before hexane (200 mL) is added. The mixture is filtered and the solid is collected and dried under vacuum to give compound 1b4.

### Step 4:

A solution of 1b4 (200 mg, 0.7 mmol) in THF (3.5 mL) is treated with NaH (60% in oil, 62 mg, 1.5 mmol). After 2 h, MeI (217 μL, 3.5 mmol) is added and the mixture is heated to 70° C. for 1 h. After being concentrated to dryness, the residue is diluted with DCM and water. The organic phase is washed with brine, dried (MgSO₄), filtered and concentrated. The residue is treated with DCM and Et₂O to give a suspension which is filtered and the solid is dried to give compound 1b5.

### Step 5:

To compound 1b5 (336 mg, 1.2 mmol) is added t-butoxybis(dimethylamino)methane (3 mL). The suspension is heated at 140° C. for 30 min before being concentrated to dryness. 2-Methoxyethylamine (3 mL) is then added and the mixture is stirred at RT for 1 h. The excess amine is removed under vacuum and the residue purified by CombiFlash (acetone/DCM) to give compound 1003.

### Example 1c

### Preparation of Compound 1007

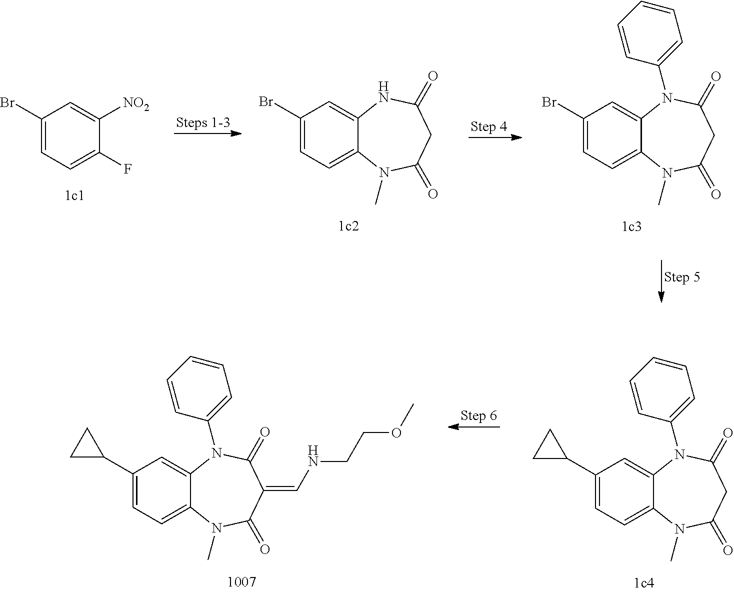 [SMILES: O=[N+]([O-])c1cc(Br)ccc1F ; CN1C(=O)CC(=O)Nc2cc(Br)ccc21 ; CN1C(=O)CC(=O)N(c2ccccc2)c2cc(Br)ccc21 ; COCCN/C=C1/C(=O)N(C)c2ccc(C3CC3)cc2N(c2ccccc2)C1=O ; CN1C(=O)CC(=O)N(c2ccccc2)c2cc(C3CC3)ccc21]

### Steps 1, 2 and 3:

Using the procedures described in steps 1-3 of example 1a, 1c1 (Aldrich) is transformed into compound 1c2, with the use of MeNH₂.HCl in place of EtNH₂.HCl.

### Step 4:

To a solution of 1c2 (500 mg, 1.86 mmol) in DCM (5 mL) at RT is added Ph₃Bi (1.31 g, 2.97 mmol), Cu(OAc)₂ (0.41 g, 2.23 mmol) and Et₃N (0.31 mL, 2.23 mmol). The reaction mixture is stirred at RT for 20 h before being concentrated onto SiO₂. The product is purified by CombiFlash (EtOAc/hexane) to afford compound 1c3.

### Step 5:

Cyclopropyl bromide (232 μL, 2.9 mmol) is dissolved in THF (8 mL) and cooled to −70° C. before a 1.6 M n-BuLi solution in hexane (1.81 mL, 2.9 mmol) is added over 5 min. After stirring for 1 h, the mixture is treated with a solution of ZnBr₂ (0.65 g, 2.9 mmol) in THF (8 mL) over 15 min. The reaction is allowed to warm slowly to RT over 1 h. A solution of 1c3 (250 mg, 0.72 mmol) in THF (2 mL) is added to the above mixture and then degassed with N₂ (10 min). The mixture is treated with Pd(PPh₃)₄ (67 mg, 0.06 mmol) and heated at 75° C. for 20 h utilizing a reflux condenser. After cooling, the mixture is partially concentrated and then extracted into EtOAc and washed with brine before being dried (MgSO₄), filtered and concentrated to give compound 1c4.

### Step 6:

Using the procedure described in step 5 of example 1b, 1c4 is transformed into compound 1007.

### Example 1d

### Preparation of Compound 1001

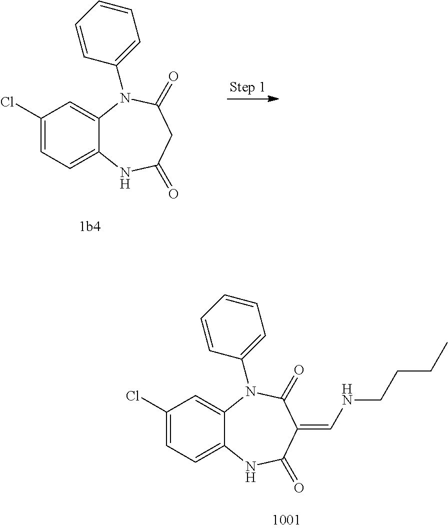 [SMILES: O=C1CC(=O)N(c2ccccc2)c2cc(Cl)ccc2N1 ; CCCCN/C=C1/C(=O)Nc2ccc(Cl)cc2N(c2ccccc2)C1=O]

### Step 1:

To an ice-cold solution of 1b4 (125 mg, 0.44 mmol) in DMF (2 mL) is slowly added PCl₅ (145 mg, 0.7 mmol) to avoid any warming. Once the addition is complete, the solution is allowed to slowly warm to RT and stirred at RT for 20 h. The mixture is cooled to 5° C. before n-butylamine is slowly added until approximately pH>8. The DMF is removed under vacuum and the residue treated with water to form a solid which is filtered and purified by preparative HPLC to give compound 1001.

### Example 1e

### Preparation of Compound 1005

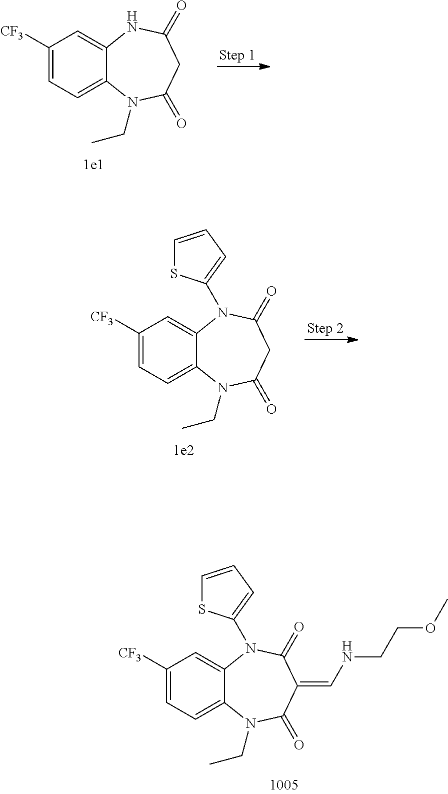 [SMILES: CCN1C(=O)CC(=O)Nc2cc(C(F)(F)F)ccc21 ; CCN1C(=O)CC(=O)N(c2cccs2)c2cc(C(F)(F)F)ccc21 ; CCN1C(=O)/C(=C/NCCOC)C(=O)N(c2cccs2)c2cc(C(F)(F)F)ccc21]

### Step 1:

A mixture of 1e1 (prepared analogously to the method described for 1a4 in example 1a; 77 mg, 0.30 mmol) in DMF (1.0 mL), K₂CO₃ (48 mg, 0.35 mmol), CuI (15 mg, 0.08 mmol), and 2-bromothiophene (130 μL, 1.36 mmol) is heated at 140° C. for 20 h. The reaction mixture is cooled and poured into aqueous 0.1 M HCl solution. The resulting mixture is extracted with EtOAc. The organic phase is washed with water and brine before being dried (MgSO₄), filtered and concentrated under vacuum. The residue is purified by flash chromatography (EtOAc:hexane) to give compound 1e2.

### Step 2:

A mixture of 1e2 (13 mg, 0.040 mmol) and t-butoxybis(dimethylamino)methane (0.7 mL) is stirred at RT for 18 h then concentrated to dryness. 2-Methoxyethylamine (50 μL, 0.57 mmol) and DMF (0.5 mL) are added and the mixture is stirred at RT for 1 h. The mixture is poured into water and extracted with EtOAc. The organic layers are washed with water, dried (MgSO4), filtered and concentrated under reduced pressure. The residue is purified by flash chromatography (2% MeOH/DCM) to give compound 1005.

### Example 2a

### Preparation of Compound 2002

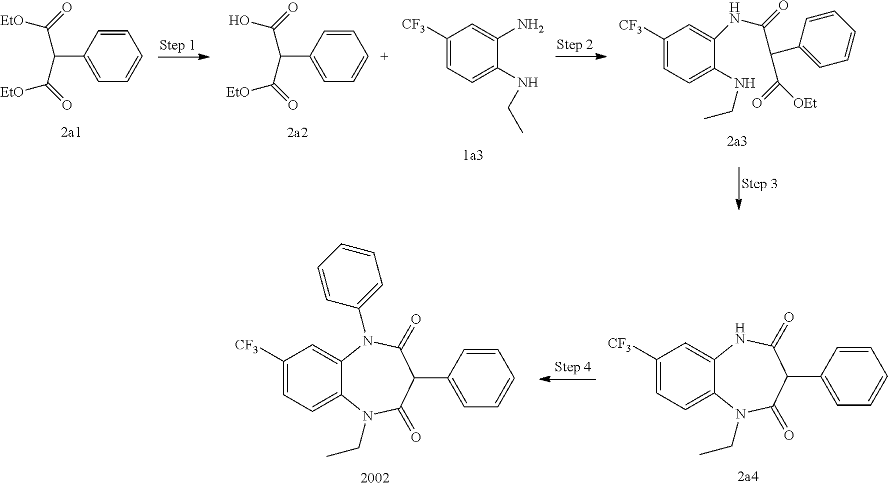 [SMILES: CCOC(=O)C(C(=O)OCC)c1ccccc1 ; CCOC(=O)C(C(=O)O)c1ccccc1 ; CCNc1ccc(C(F)(F)F)cc1N ; CCNc1ccc(C(F)(F)F)cc1NC(=O)C(C(=O)OCC)c1ccccc1 ; CCN1C(=O)C(c2ccccc2)C(=O)N(c2ccccc2)c2cc(C(F)(F)F)ccc21 ; CCN1C(=O)C(c2ccccc2)C(=O)Nc2cc(C(F)(F)F)ccc21]

### Step 1:

Aqueous 1.0 N NaOH solution (23.0 mL, 23.0 mmol) is added to a solution of 2a1 (Aldrich; 5.00 g, 21.2 mmol) in EtOH (20 mL). The reaction mixture is stirred at RT for 3 h. The mixture is concentrated under reduced pressure and the residual aqueous solution is neutralized by addition of aqueous 1.0 N HCl solution (22 mL) and pH 7 buffer is added. The mixture is extracted with EtOAc. The organic layer is washed with water and brine, dried (MgSO₄), filtered and concentrated under reduced pressure to give compound 2a2.

### Step 2:

A solution of 2a2 (1.12 g, 5.38 mmol), 1a3 (1.10 g, 5.38 mmol), HATU (2.46 g, 6.46 mmol) and i-Pr₂NEt (2.34 mL, 13.4 mmol) in DCM (75 mL) is stirred at RT for 16 h. The mixture is diluted with DCM and the solution is successively washed with aqueous 1.0 N HCl solution, water and brine, dried (MgSO₄), filtered and concentrated under reduced pressure. The residue is purified by flash chromatography (DCM:acetone, 19:1) to give compound 2a3.

### Step 3:

A solution of 1.0 M NaHMDS in THF (3.25 mL, 3.25 mmol) is added to a solution of 2a3 (1.16 g, 2.95 mmol) in DMF (15 mL) at RT. The mixture is stirred at RT for 1 h. Water and EtOAc are added. The organic layer is washed with aqueous 1.0 N HCl solution, water and brine, dried (MgSO₄), filtered and concentrated under reduced pressure. The residue is purified by flash chromatography (DCM:acetone, 19:1) to give compound 2a4.

### Step 4:

Ph₃Bi (255 mg, 574 μmol), Cu(OAc)₂ (104 mg, 574 μmol) and pyridine (46.4 μL, 574 μmol) are added to a solution of 2a4 (100 mg, 287 μmol) in DCM (15 mL) at RT. The mixture is stirred at RT for 16 h. The reaction mixture is diluted with DCM and the solution is washed with aqueous 1.0 N HCl solution, water and brine, dried (MgSO₄), filtered and concentrated under reduced pressure. The residue is purified by preparative HPLC to give compound 2002.

### Example 2b

### Preparation of Compound 2003

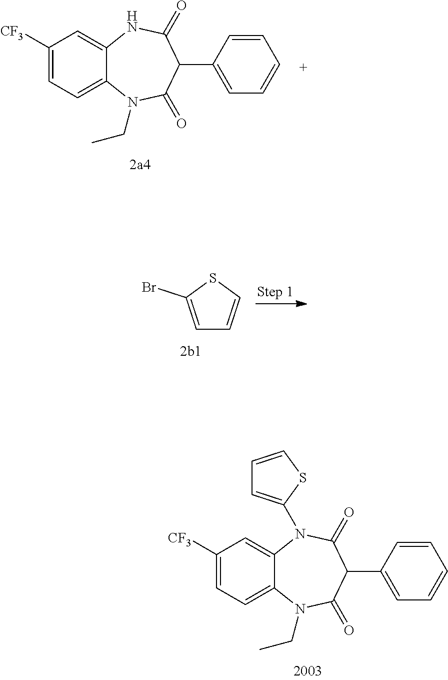 [SMILES: CCN1C(=O)C(c2ccccc2)C(=O)Nc2cc(C(F)(F)F)ccc21 ; Brc1cccs1 ; CCN1C(=O)C(c2ccccc2)C(=O)N(c2cccs2)c2cc(C(F)(F)F)ccc21]

### Step 1:

Using the procedure described in step 1 of example 1e, 2a4 is transformed into compound 2003 using 2b1 (Aldrich).

### Example 2c

### Preparation of Compounds 2008 and 2010

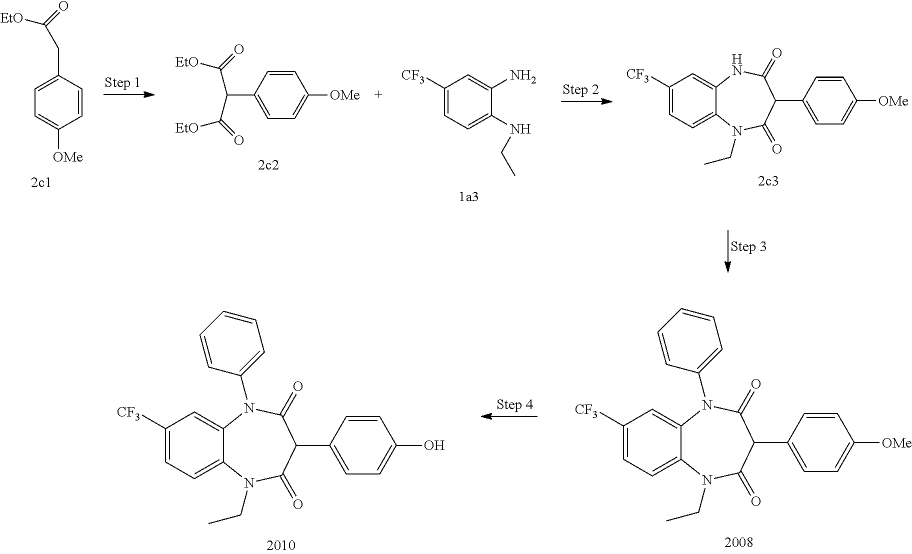 [SMILES: CCOC(=O)Cc1ccc(OC)cc1 ; CCOC(=O)C(C(=O)OCC)c1ccc(OC)cc1 ; CCNc1ccc(C(F)(F)F)cc1N ; CCN1C(=O)C(c2ccc(OC)cc2)C(=O)Nc2cc(C(F)(F)F)ccc21 ; CCN1C(=O)C(c2ccc(O)cc2)C(=O)N(c2ccccc2)c2cc(C(F)(F)F)ccc21 ; CCN1C(=O)C(c2ccc(OC)cc2)C(=O)N(c2ccccc2)c2cc(C(F)(F)F)ccc21]

### Step 1:

A solution of 1.0 M LiHMDS in THF (43.2 mL, 43.2 mmol) is added to a cold (−78° C.) solution of 2c1 (Aldrich; 7.00 g, 36.0 mmol) in THF (300 mL). The reaction mixture is stirred at this temperature for 1 h. Ethyl cyanoformate (5.34 mL, 43.2 mmol) is added and the reaction mixture is stirred at −78° C. for 30 min then allowed to warm to RT and maintained at this temperature for 3 h. Aqueous NH₄Cl solution is added and the mixture stirred at RT for 30 min. The mixture then is concentrated under reduced pressure, EtOAc is added and the phases are separated. The organic layer is washed with water and brine, dried (MgSO₄), filtered and concentrated under reduced pressure. The residue is purified by flash chromatography (DCM) to give compound 2c2.

### Step 2:

A solution of 1a3 (1.50 g, 7.35 mmol) and 2c2 (1.96 g, 7.35 mmol) in DMF (8.0 mL) is heated at 170° C. for 25 min in a microwave. The mixture is cooled to RT and a solution of 1.0 M NaHMDS in THF (14.7 mL, 14.7 mmol) is added and the mixture is stirred at RT for 30 min. Aqueous 1.0 N HCl solution is added to the mixture. EtOAc is added and the phases are separated. The organic layer is washed with water and brine, dried (MgSO₄), filtered and concentrated under reduced pressure. The residue is purified by flash chromatography (DCM:acetone, 19:1) to give compound 2c3.

### Step 3:

Using the procedure described in step 4 of example 2a, compound 2c3 is transformed into compound 2008.

### Step 4:

A solution of compound 2008 (20.0 mg, 44.0 μmol) and a 48% HBr solution in AcOH (1.0 mL) in AcOH (1.0 mL) is stirred at 100° C. for 3 h. The cooled reaction mixture is diluted with water and carefully neutralized by addition of aqueous 1.0 N NaOH solution. The mixture is extracted with EtOAc. The combined organic layers are washed with water and brine, dried (MgSO₄), filtered and concentrated under reduced pressure. The residue is purified by preparative HPLC to give compound 2010.

### Example 2d

### Preparation of Compound 2025

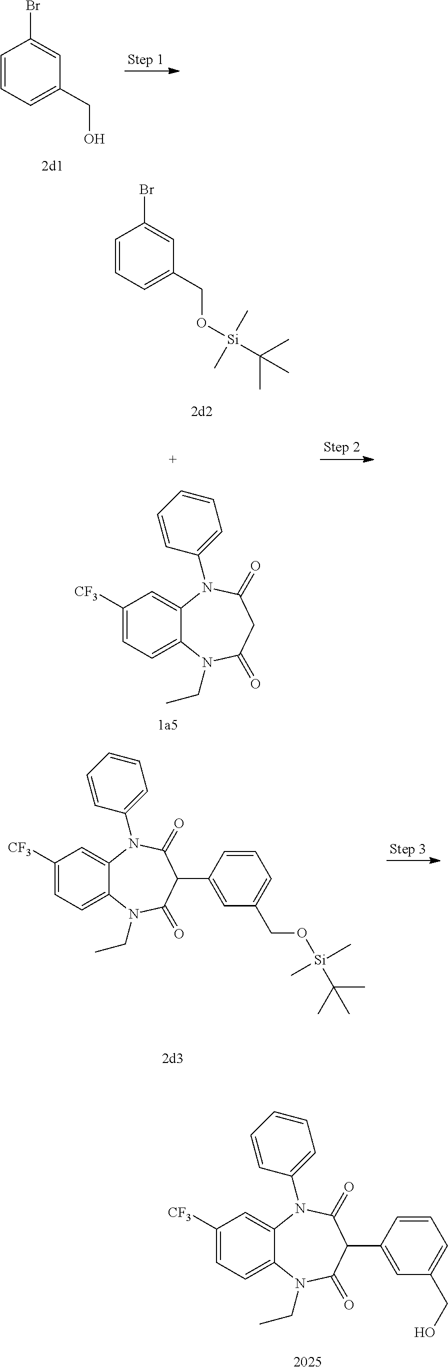 [SMILES: OCc1cccc(Br)c1 ; CC(C)(C)[Si](C)(C)OCc1cccc(Br)c1 ; CCN1C(=O)CC(=O)N(c2ccccc2)c2cc(C(F)(F)F)ccc21 ; CCN1C(=O)C(c2cccc(CO[Si](C)(C)C(C)(C)C)c2)C(=O)N(c2ccccc2)c2cc(C(F)(F)F)ccc21 ; CCN1C(=O)C(c2cccc(CO)c2)C(=O)N(c2ccccc2)c2cc(C(F)(F)F)ccc21]

### Step 1:

TBDMS-Cl (2.9 g, 19 mmol) is added to a solution of 2d1 (Lancaster, 2.0 g, 11 mmol) and imidazole (1.5 g, 21 mmol) in DCM (35 mL) and the reaction is stirred at RT for 1 h. The reaction is quenched with water and the layers separated. The aqueous layer is extracted with DCM, dried over Na₂SO₄, filtered and concentrated under reduced pressure. The crude product is purified by CombiFlash to give compound 2d2.

### Step 2:

A solution of 1a5 (75.0 mg, 0.215 mmol) and (t-Bu)₃P (11.6 μL, 43 μmol) in THF (2.0 mL) is added to K₃PO₄ (141 mg, 0.646 mmol) under a N₂ atmosphere. A solution of (t-Bu₃P)₂Pd (22.5 mg, 43 mmol) in THF (1.0 mL) and a solution of 2d2 (71.3 mg, 0.237 mmol) in THF (1.0 mL) are successively added to the mixture. The reaction mixture is heated at 80° C. for 16 h. Additional amounts of (t-Bu)₃P (11.6 μL, 43 mmol) and (t-Bu₃P)₂Pd (22.5 mg, 43 are added and the mixture is stirred at 80° C. for 16 h. The cooled mixture diluted with EtOAc is washed with water and brine, dried (MgSO₄), filtered and concentrated under reduced pressure. The residue is purified by flash chromatography (hexane:EtOAc, 7:3) to give compound 2d3.

### Step 3:

A solution of 2d3 (54.0 mg, 95.0 mmol) and a 1.0 M TBAF solution in THF (95 μL, 95 mmol) in THF (0.8 mL) containing AcOH (11 μL) is stirred at 0° C. for 10 min and then stirred at RT for 16 h. The mixture is concentrated under reduced pressure and the residue is purified by preparative HPLC to give compound 2025.

### Example 2e

### Preparation of Compound 2059

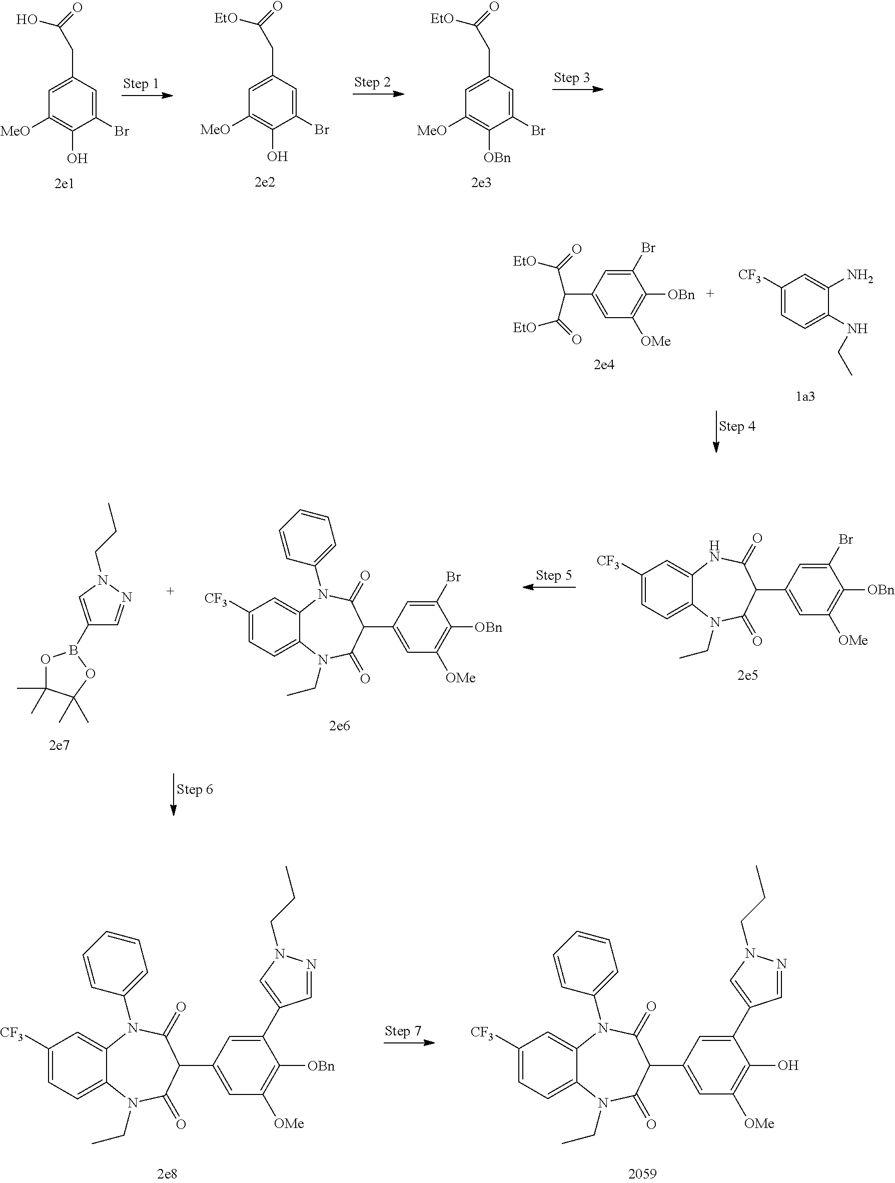

### Step 1:

A solution of 2e1 (Transworld; 2.50 g, 9.58 mmol) and concentrated H₂SO₄ (0.5 g) in EtOH (94 mL) is heated to reflux for 6 h. The mixture is cooled to RT and diluted with water and brine. The mixture is extracted with EtOAc. The organic layer is washed with water, dried (Na₂SO₄), filtered and concentrated under reduced pressure to give compound 2e2.

### Step 2:

A mixture of 2e2 (3.06 g, 9.63 mmol), BnBr (1.76 mL, 14.4 mmol) and K₂CO₃ (2.66 g, 19.3 mmol) in DMF (10.3 mL) is stirred at RT for 16 h. The mixture diluted with water is extracted with EtOAc. The combined organic layers are washed with water, dried (MgSO₄), filtered and concentrated under reduced pressure. The residue is purified by CombiFlash to give compound 2e3.

### Step 3:

Using the procedure described in step 1 of example 2c, 2e3 is transformed into compound 2e4.

### Step 4:

Using the procedure described in step 2 of example 2c, 2e4 is transformed into compound 2e5.

### Step 5:

Using the procedure described in step 4 of example 2a, 2e5 is transformed into compound 2e6.

### Step 6:

A mixture of 2e6 (191 mg, 299 μmol), 2e7 (Aldrich; 141 mg, 597 μmol), (t-Bu₃P)₂Pd (38.1 mg, 74.7 mmol) and aqueous 2.0 M Na₂CO₃ solution (149 μL, 299 μmol) in DMF (6.9 mL) is heated to 145° C. for 15 min in a microwave. The cooled mixture diluted with brine is extracted with EtOAc. The combined organic layers are washed with water, dried (MgSO₄), filtered and concentrated under reduced pressure. The residue is purified by CombiFlash to give compound 2e8.

### Step 7:

A mixture of 2e8 (24.0 mg, 36 μmol) and 10% Pd/C (21 mg) in DMF (53 μL) and EtOH (0.5 mL) is stirred under a H₂ atmosphere (1 atm) for 1 h. The mixture is filtered through Celite®. The filtrate is concentrated under reduced pressure and the residue purified by preparative HPLC to give compound 2059.

### Example 2f

### Preparation of Compounds 2015 and 2030

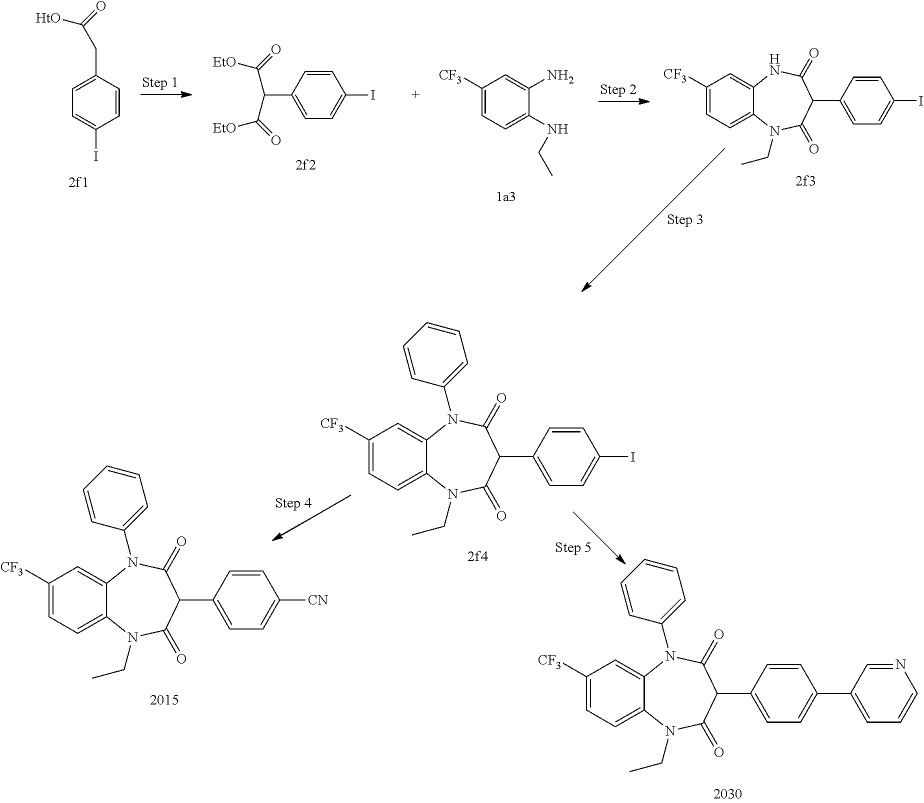

### Step 1:

A solution of 2f1 (Lancaster; 4.54 g, 17.3 mmol) and concentrated HCl in EtOH (100 mL) is heated to reflux for 16 h. The cooled mixture is concentrated under reduced pressure and the residue diluted with EtOAc. The solution is washed with aqueous saturated NaHCO₃ solution, water and brine, dried (MgSO₄), filtered and concentrated under reduced pressure. The resulting ethyl ester is transformed into compound 2f2 using the procedure described in step 1 of example 2c.

### Step 2:

Using the procedure described in step 2 of example 2c, 2f2 is transformed into compound 2f3.

### Step 3:

Using the procedure described in step 4 of example 2a, 2f3 is transformed into compound 2f4.

### Step 4:

A mixture of 2f4 (50.0 mg, 90.8 μmol) and CuCN (40.7 mg, 454 μmol) in DMF (3.0 mL) is heated to 190 C for 20 min in a microwave. The cooled mixture is diluted with EtOAc and the resulting solution is washed with aqueous 1.0 N HCl solution, water and brine, dried (MgSO₄), filtered and concentrated under reduced pressure. The residue is purified by preparative HPLC to give compound 2015.

### Step 5:

A mixture of 2f4 (60.0 mg, 109 mmol), pyridine-3-boronic acid (Lancaster, 40.2 mg, 327 μmol), (t-Bu₃P)₂Pd (11.9 mg, 27.2 μmol), aqueous 2.0 M Na₂CO₃ solution (0.22 mL, 0.44 mmol) in DMF (1.5 mL) is heated to 125° C. for 15 min in a microwave. The cooled mixture is diluted with EtOAc and the resulting solution is washed with water and brine, dried (MgSO₄), filtered and concentrated under reduced pressure. The residue is purified by preparative HPLC to give compound 2030.

### Example 2g

### Preparation of Compounds 2046 and 2050

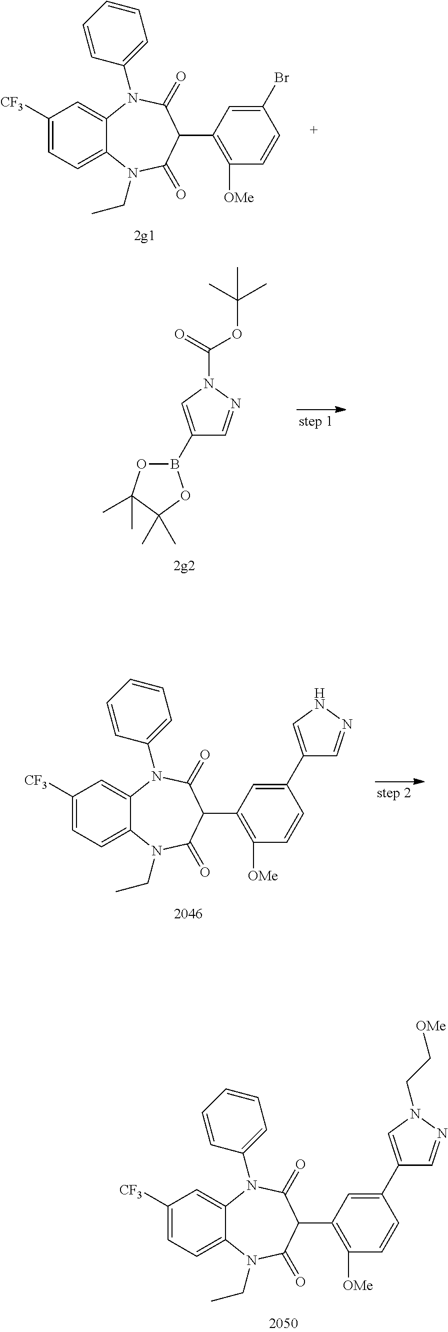 [SMILES: CCN1C(=O)C(c2cc(Br)ccc2OC)C(=O)N(c2ccccc2)c2cc(C(F)(F)F)ccc21 ; CC(C)(C)OC(=O)n1cc(B2OC(C)(C)C(C)(C)O2)cn1 ; CCN1C(=O)C(c2cc(-c3cn[nH]c3)ccc2OC)C(=O)N(c2ccccc2)c2cc(C(F)(F)F)ccc21 ; CCN1C(=O)C(c2cc(-c3cnn(CCOC)c3)ccc2OC)C(=O)N(c2ccccc2)c2cc(C(F)(F)F)ccc21]

### Step 1:

A mixture of 2g1 (prepared analogously to the method described for 2e6 in example 2e; 600 mg, 1.12 mmol), 2g2 (Boron Molecular Ltd; 993 mg, 3.37 mmol), (t-Bu₃P)₂Pd (172 mg, 338 μmol I) and aqueous 2.0 M Na₂CO₃ solution (2.8 mL, 5.6 mmol) in DMF (12 mL) is heated to 125° C. for 17 min in a microwave. The cooled mixture is filtered and diluted with EtOAc. The solution is washed with aqueous 1 N HCl solution, water and brine, dried (MgSO₄), filtered and concentrated under reduced pressure. The residue is purified by flash chromatography (DCM:MeOH, 9:1) to give compound 2046.

### Step 2:

A solution of 1.0 M LiHMDS in THF (154 μL, 154 μmol) is added to a solution of compound 2046 (40.0 mg, 76.8 mmol) and 2-bromoethyl methyl ether (7.94, 84 μmol) in DMF (2.0 mL) at RT. The mixture is stirred at RT for 1 h then aqueous 1 N HCl solution and EtOAc are added. The resulting solution is washed with water and brine, dried (MgSO₄), filtered and concentrated under reduced pressure. The residue is dissolved in DCM (5.0 mL) and cooled to 0° C. A solution of 1.0 M BBr₃ in DCM (154 μL, 154 mmol) is added and the mixture is stirred at 0° C. for 45 min.

Aqueous 1 N HCl solution is added and the mixture is diluted with DCM. The solution is washed with aqueous 1 N HCl solution, water and brine, dried (MgSO₄), filtered and concentrated under reduced pressure. The residue is purified by preparative HPLC to give compound 2050.

### Example 2h

### Preparation of Compound 2011

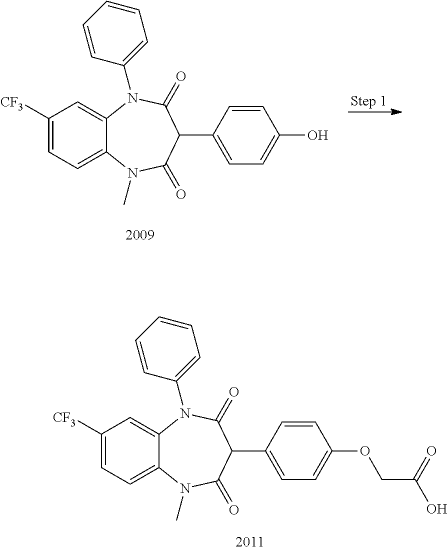 [SMILES: CN1C(=O)C(c2ccc(O)cc2)C(=O)N(c2ccccc2)c2cc(C(F)(F)F)ccc21 ; CN1C(=O)C(c2ccc(OCC(=O)O)cc2)C(=O)N(c2ccccc2)c2cc(C(F)(F)F)ccc21]

### Step 1:

NaH (60% in oil; 11.2 mg, 281 μmol) is added to a solution of compound 2009 (prepared analogously to the method described for 2010 in example 2c; 40.0 mg, 93.8 mmol) in THF (3.0 mL). The mixture is stirred at RT for 30 min and tert-butyl bromoacetate is added. The reaction mixture is then stirred at RT for 16 h. Aqueous 1 N HCl is added and the mixture diluted with EtOAc is washed with water and brine, dried (MgSO₄), filtered and concentrated under reduced pressure. The crude ester is dissolved in DCM (3.0 mL) and TFA (1.0 mL) is added. The mixture is stirred at RT for 1 h then is concentrated under reduced pressure. The residue is purified by preparative HPLC to give compound 2011.

### Example 3a

### Preparation of Compounds 3001 and 3002

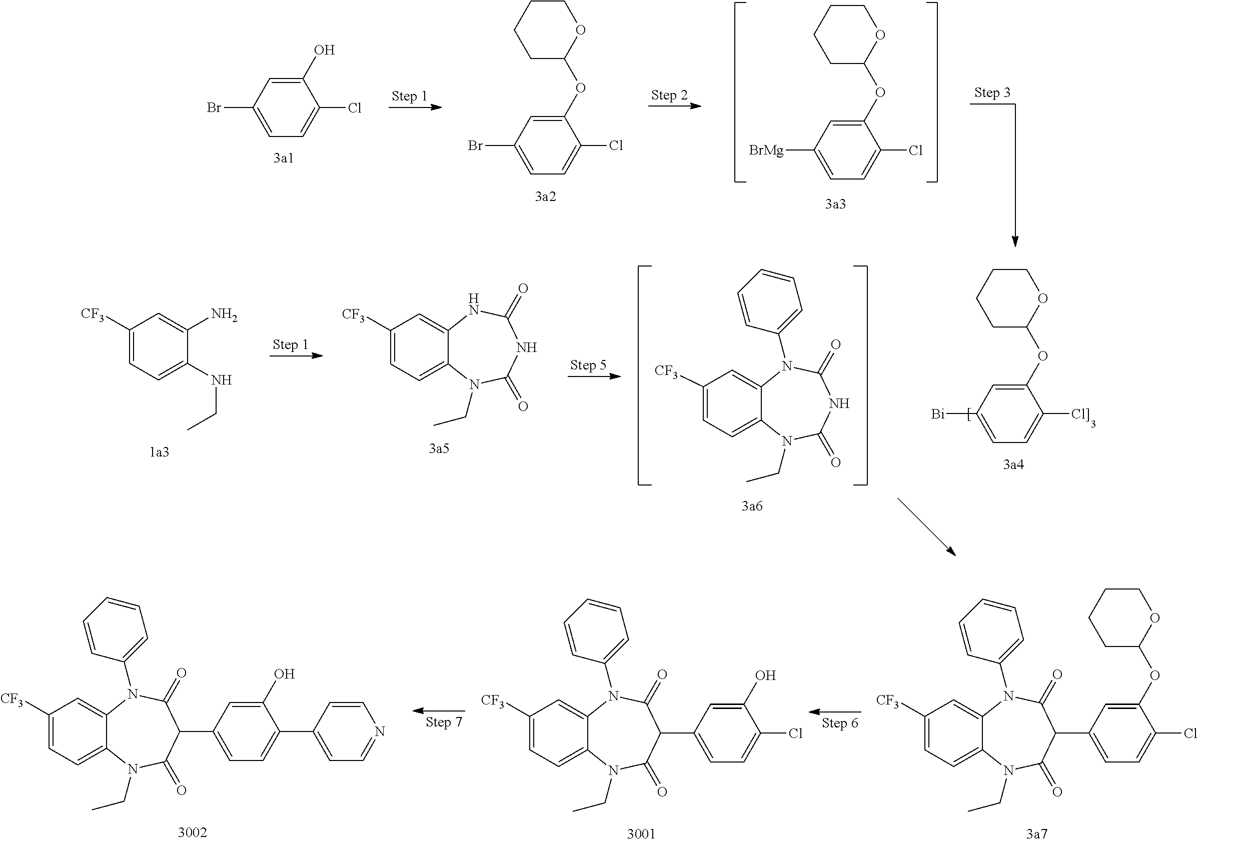

### Step 1:

Solid pTsOH.H₂O (100 mg, 0.5 mmol) is added to a mixture of 3a1 (Alfa; 5.0 g, 24 mmol) and dihydropyrane (3.0 mL, 33 mmol) and the mixture is stirred for 24 h. The reaction is diluted with DCM, silica gel is added and the mixture is concentrated to dryness. The product is purified by Combiflash to give compound 3a2.

### Step 2:

Solid I₂ (1 mg, 0.008 mmol) is added to a mixture of Mg turnings (1.6 g, 65 mmol) and compound 3a2 (4.8 mL, 16 mmol) in anhydrous THF (30 mL). The mixture is heated to reflux for 30 min. The reaction solution is then transferred to a clean, dry flask to give compound 3a3 as a solution in THF.

### Step 3:

A solution of 3a3 (0.30 M, 30 mL, 16 mmol) is added to a suspension of BiCl₃ (1.0 g, 3.3 mmol) under a N₂ atmosphere at 0° C. and the above solution is allowed to warm to RT and stir overnight. The reaction is poured into water and extracted with EtOAc. The organic layer is washed with brine, dried over MgSO₄ and concentrated under reduced pressure. The residue is purified by Combiflash to give compound 3a4.

### Step 4:

A solution of phenyl isocyanatoformate (2.40 g, 14.7 mmol) in DMF (20 mL) is added dropwise to a solution of compound 1a3 (2.10 g, 10.3 mmol) in DMF (170 mL) at RT. After the addition is complete, the reaction is heated to 90° C. for 16 h. The reaction is cooled to RT, diluted with brine and extracted with EtOAc. The organic layer is dried over MgSO₄, filtered and evaporated to dryness. The product is purified by Combiflash followed by trituration with EtOAc/hexane to give compound 3a5.

### Step 5:

Ph₃Bi (300 mg, 0.7 mmol) is added to a suspension of compound 3a5 (130 mg, 0.46 mmol), Cu(OAc)₂ (130 mg, 0.70 mmol) and pyridine (75 μL, 0.93 mmol) in 1,4-dioxane (2.2 mL). The mixture is heated to 80° C. and is stirred for 2 h to give a solution containing 3a6. A solution of 3a4 (590 mg, 0.70 mmol) in 1,4-dioxane is added to the reaction. Solid Cu(OAc)₂ (130 mg, 0.70 mmol) followed by pyridine (75 μL, 0.93 mmol) are added to the reaction and stirring is continued at 80° C. for 3 h. The reaction is cooled to RT and diluted with DCM. Silica gel is added and the mixture is concentrated to dryness. The product is purified by Combiflash to give compound 3a7.

### Step 6:

Compound 3a7 (30 mg, 0.05 mmol) is dissolved in THF/TFA/water (4:2:1, 1.4 mL) and the solution is stirred for 3 h at RT. The mixture is diluted with DCM, silica gel is added and the mixture is concentrated. The mixture is purified by Combiflash to give compound 3001.

### Step 7:

Compound 3001 (33 mg, 0.07 mmol), pyridine-4-boronic acid (Aldrich, 17 mg, 0.14 mmol) and ((t-Bu)₃P)₂Pd (11 mg, 21 mmol) are dissolved in a mixture of aqueous Na₂CO₃ solution (0.17 mL, 0.34 mmol) and DMF (1 mL). The reaction mixture is heated to 145° C. for 10 min in a microwave. The reaction is diluted with brine and extracted with EtOAc. The organic layer is washed with brine and concentrated.

The mixture is purified by Combiflash to give compound 3002.

### Example 3b

### Preparation of Compound 3004

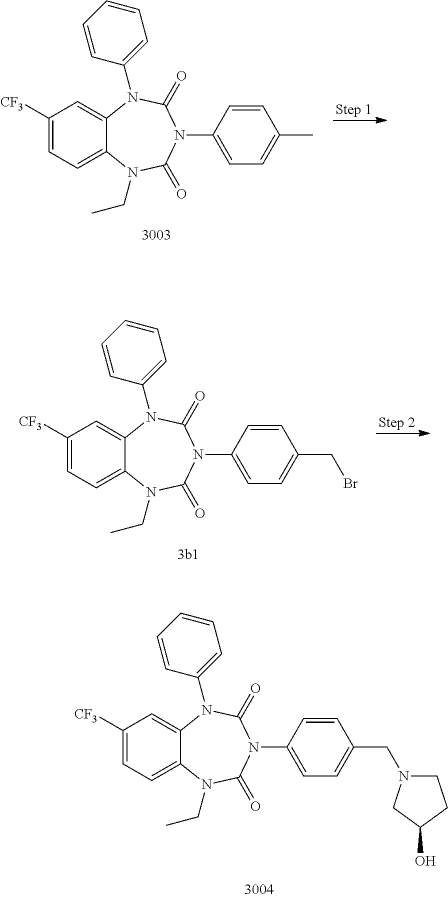 [SMILES: CCN1C(=O)N(c2ccc(C)cc2)C(=O)N(c2ccccc2)c2cc(C(F)(F)F)ccc21 ; CCN1C(=O)N(c2ccc(CBr)cc2)C(=O)N(c2ccccc2)c2cc(C(F)(F)F)ccc21 ; CCN1C(=O)N(c2ccc(CN3CC[C@@H](O)C3)cc2)C(=O)N(c2ccccc2)c2cc(C(F)(F)F)ccc21]

### Step 1:

Compound 3003 is prepared analogously to the procedure described for compound 3a7 following steps 2 to 5 of example 3a, except that p-bromotoluene is used in place of 3a2. A solution of compound 3003 (520 mg, 1.2 mmol) in CCl₄ (8 mL) is treated sequentially with AIBN (50 mg, 0.3 mmol) and NBS (210 mg, 1.2 mmol) and this mixture is heated to reflux for 1 h. A portion of NBS (110 mg, 0.60 mmol) is added and the reaction is continued for 1 h. The reaction mixture is concentrated to dryness and the residue purified by Combiflash to give compound 3b1.

### Step 2:

(R)-3-pyrrolidinol hydrochloride (12 mg, 0.096 mmol) and Et₃N (27 μL, 0.19 mmol) are added to a solution of 3b1 (25 mg, 0.048 mmol) in DMF (500 μL) and the mixture is stirred for 2 h. The reaction is diluted with AcOH (500 μL) and the mixture purified by preparative HPLC to give compound 3004.

### Example 3c

### Preparation of Compounds 3006 and 3007

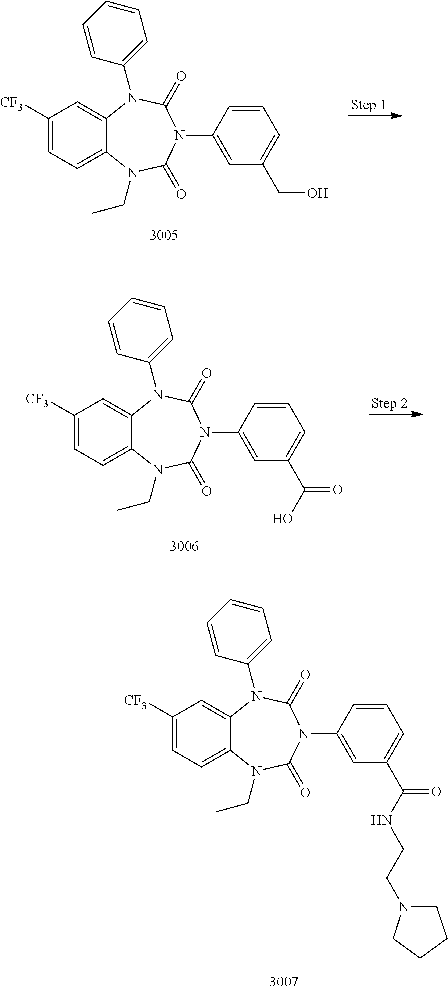 [SMILES: CCN1C(=O)N(c2cccc(CO)c2)C(=O)N(c2ccccc2)c2cc(C(F)(F)F)ccc21 ; CCN1C(=O)N(c2cccc(C(=O)O)c2)C(=O)N(c2ccccc2)c2cc(C(F)(F)F)ccc21 ; CCN1C(=O)N(c2cccc(C(=O)NCCN3CCCC3)c2)C(=O)N(c2ccccc2)c2cc(C(F)(F)F)ccc21]

### Step 1:

Compound 3005 is prepared analogously to the procedure described for compound 3a7 following steps 1 to 6 of example 3a, except that m-bromobenzyl alcohol is used in place of 5-bromo-2-chlorophenol. A solution of compound 3005 (15 mg, 0.033 mmol) in acetone (0.5 mL) is treated with a solution of CrO₃ in 20% v/v H₂SO₄/water (0.3 M, 230 μL, 0.70 mmol). Upon complete oxidation, the reaction is diluted with water and extracted with EtOAc. The organic layer is dried over Na₂SO₄ and concentrated and the residue triturated with EtOAc/hexane to give compound 3006.

### Step 2:

Et₃N (25 μL, 0.16 mmol) is added to a solution of compound 3006 (16 mg, 0.035 mmol) and TBTU (26 mg, 0.080 mmol) dissolved in DMF (500 uL) and the reaction mixture is stirred for 10 min at RT. A solution of N-(2-aminoethyl)pyrrolidine (20 mg, 0.18 mmol) in THF (500 μL) is added and the reaction mixture is stirred for 2 h. The mixture is diluted with DMSO (1 mL) and the mixture purified by preparative HPLC to give compound 3007.

### Example 3d

### Preparation of Compound 3009

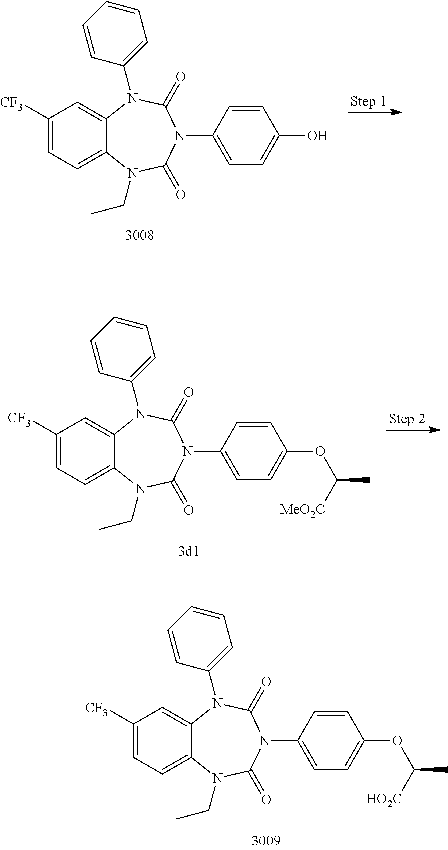 [SMILES: CCN1C(=O)N(c2ccc(O)cc2)C(=O)N(c2ccccc2)c2cc(C(F)(F)F)ccc21 ; CCN1C(=O)N(c2ccc(O[C@@H](C)C(=O)OC)cc2)C(=O)N(c2ccccc2)c2cc(C(F)(F)F)ccc21 ; CCN1C(=O)N(c2ccc(O[C@@H](C)C(=O)O)cc2)C(=O)N(c2ccccc2)c2cc(C(F)(F)F)ccc21]

### Step 1:

Compound 3008 is prepared analogously to the procedure described for compound 3a7 following steps 1 to 5 of example 3a, except that p-bromophenol is used in place of 5-bromo-2-chlorophenol. Neat DIAD (16 μL, 0.082 mmol) is added to a solution of compound 3008 (30 mg, 0.07 mmol), methyl (R)-lactate (9 mg, 0.08 mmol) and Ph₃P (22 mg, 0.082 mmol) in THF (1.5 mL) and the reaction mixture is stirred for 16 h. Additional amounts of Ph₃P (22 mg, 0.082 mmol) and DIAD (16 μL, 0.082 mmol) are added sequentially and the reaction is stirred for 16 h. The mixture is concentrated and the residue purified by Combiflash to give compound 3d1.

### Step 2:

An aqueous 1.0 N LiOH solution (1.0 mL, 1.0 mmol) is added to a solution of compound 3d1 (56 mg, 0.10 mmol) in THF/water (1:1, 6.5 mL) and the mixture is stirred at RT for 45 min. The reaction mixture is diluted with water and acidified with aqueous 1N HCl solution (300 μL). The mixture is extracted with EtOAc, dried over MgSO₄ and concentrated to dryness. The residue is then purified by Combiflash to give compound 3009.

### Example 3e

### Preparation of Compound 3010

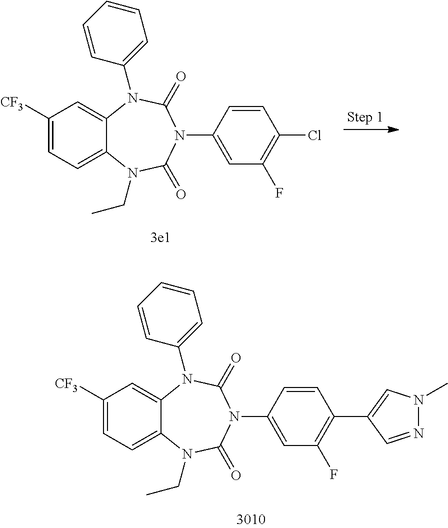 [SMILES: CCN1C(=O)N(c2ccc(Cl)c(F)c2)C(=O)N(c2ccccc2)c2cc(C(F)(F)F)ccc21 ; CCN1C(=O)N(c2ccc(-c3cnn(C)c3)c(F)c2)C(=O)N(c2ccccc2)c2cc(C(F)(F)F)ccc21]

### Step 1:

Compound 3e1 is prepared analogously to the procedure described for compound 3a7 following steps 2 to 5 of example 3a, except that 1-bromo-4-chloro-3-fluorobenzene is used in place of 3a2. Compound 3010 is prepared from compound 3e1 as described for the preparation of compound 3002 following step 7 of example 3a.

### Example 3f

### Preparation of Compound 3066

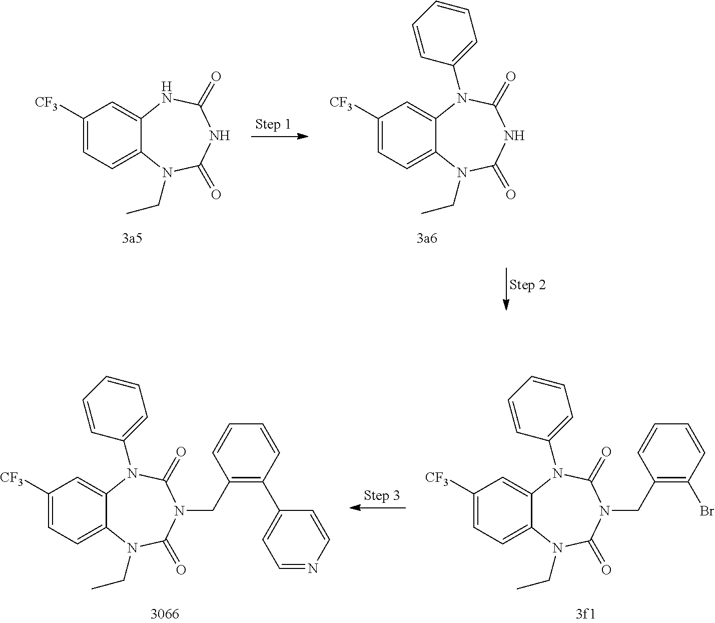 [SMILES: CCN1C(=O)NC(=O)Nc2cc(C(F)(F)F)ccc21 ; CCN1C(=O)NC(=O)N(c2ccccc2)c2cc(C(F)(F)F)ccc21 ; CCN1C(=O)N(Cc2ccccc2-c2ccncc2)C(=O)N(c2ccccc2)c2cc(C(F)(F)F)ccc21 ; CCN1C(=O)N(Cc2ccccc2Br)C(=O)N(c2ccccc2)c2cc(C(F)(F)F)ccc21]

### Step 1:

Compound 3a6 is prepared from compound 3a5 analogously to the procedure described in step 5 of example 3a, except that the solution of 3a6 is cooled to RT, diluted with DCM and SiO₂ gel is added. Compound 3a6 is then purified by Combiflash followed by trituration with EtOAc/hexane.

### Step 2:

K₃PO₄ (36 mg, 0.17 mmol) followed by 2-bromobenzyl bromide (42 mg, 0.17 mmol) are added to a solution of 3a6 (30 mg, 0.09 mmol) in DMF (1.2 mL) and the reaction mixture is heated to 80° C. and stirred overnight. The reaction mixture is diluted with EtOAc and water and the organic layer is washed with water. The organic layer is dried over Na₂SO₄ and concentrated. The residue is purified by Combiflash to give 3f1.

### Step 3:

Solid Pd[P(t-Bu)₃P]₂ (7 mg, 0.01 mmol), pyridine-4-boronic acid (Aldrich, 12 mg, 0.10 mmol) and a solution of sodium carbonate in water (2 M, 100 μL, 0.2 mmol) are added to a solution of 3f1 (30 mg, 0.06 mmol) in DMF (1.4 mL). The mixture is heated to 145° C. under microwave irradiation for 15 min then cooled to RT, diluted with water and extracted with EtOAc. The organic layer is washed with water, dried over Na₂SO₄, concentrated and the products are separated by Combiflash to give compound 3066.

### Example 4a

### Preparation of Compounds 4001 and 6008

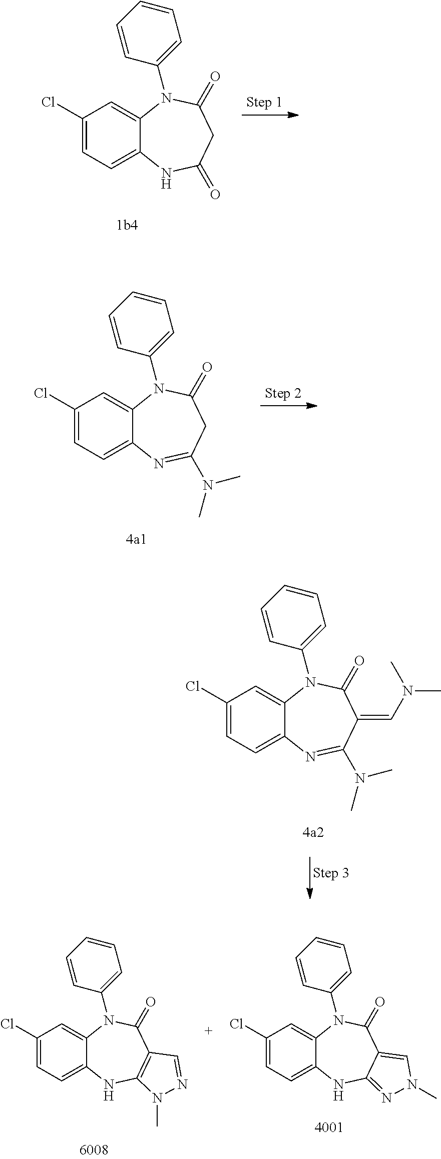 [SMILES: O=C1CC(=O)N(c2ccccc2)c2cc(Cl)ccc2N1 ; CN(C)C1=Nc2ccc(Cl)cc2N(c2ccccc2)C(=O)C1 ; CN(C)/C=C1\C(=O)N(c2ccccc2)c2cc(Cl)ccc2N=C1N(C)C ; Cn1ncc2c1Nc1ccc(Cl)cc1N(c1ccccc1)C2=O ; Cn1cc2c(n1)Nc1ccc(Cl)cc1N(c1ccccc1)C2=O]

### Step 1:

To a 2.0 M Me₂NH solution in THF (6.3 mL, 12.6 mmol) at 0° C. is added compound 1b4 (200 mg, 0.7 mmol) followed by a solution of TiCl₄ (123 μL, 1.12 mmol) in toluene (4.5 mL). The reaction mixture is stirred at RT for 4 h, then diluted with EtOAc and brine and is filtered through Celite®. The filtrate is concentrated and the crude product is purified by flash chromatography (EtOAc:hexane, 1:1) to give compound 4a1.

### Step 2:

A solution of 4a1 (67 mg, 0.21 mmol) in Bredereck's reagent (2.0 mL, 9.6 mmol) is stirred at 95° C. for 7 days. The reaction mixture is evaporated to dryness then diluted with DCM, washed with water and brine, dried (MgSO₄), and concentrated under vacuum to give crude 4a2.

### Step 3:

A mixture of 4a2 (89 mg, 0.24 mmol), methylhydrazine (16.6 mg, 0.36 mmol) and AcOH (121 μL) in n-BuOH (1.2 mL) is heated to reflux for 1 h. The reaction mixture is evaporated to dryness then diluted with DCM washed with an aqueous 10% Na₂CO₃ solution and brine, dried (MgSO₄), and concentrated under vacuum. The mixture of the 2 isomers is separated by flash chromatography (EtOAc:hexane, 4:1) to give compounds 6008 and 4001.

### Example 4b

### Preparation of Compound 4002

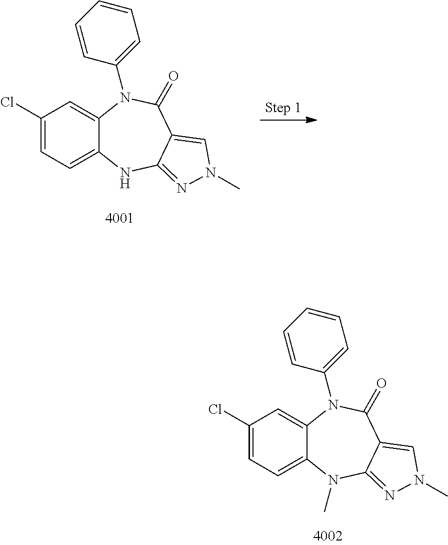 [SMILES: Cn1cc2c(n1)Nc1ccc(Cl)cc1N(c1ccccc1)C2=O ; CN1c2ccc(Cl)cc2N(c2ccccc2)C(=O)c2cn(C)nc21]

### Step 1:

To a solution of 4001 (40 mg, 0.12 mmol) in THF (1 mL) is added NaH (60% dispersion, 5.4 mg, 0.14 mmol). The mixture is stirred at RT for 1 h, and then MeI (39 μL, 0.62 mmol) is added. The reaction mixture is heated to 70° C. for 16 h, cooled to RT and evaporated to dryness. The residue diluted with DCM is washed with water and brine, dried (MgSO₄), and concentrated under vacuum. The residue is purified by preparative HPLC to give, after lyophilization, compound 4002.

### Example 4c

### Preparation of Compound 4003

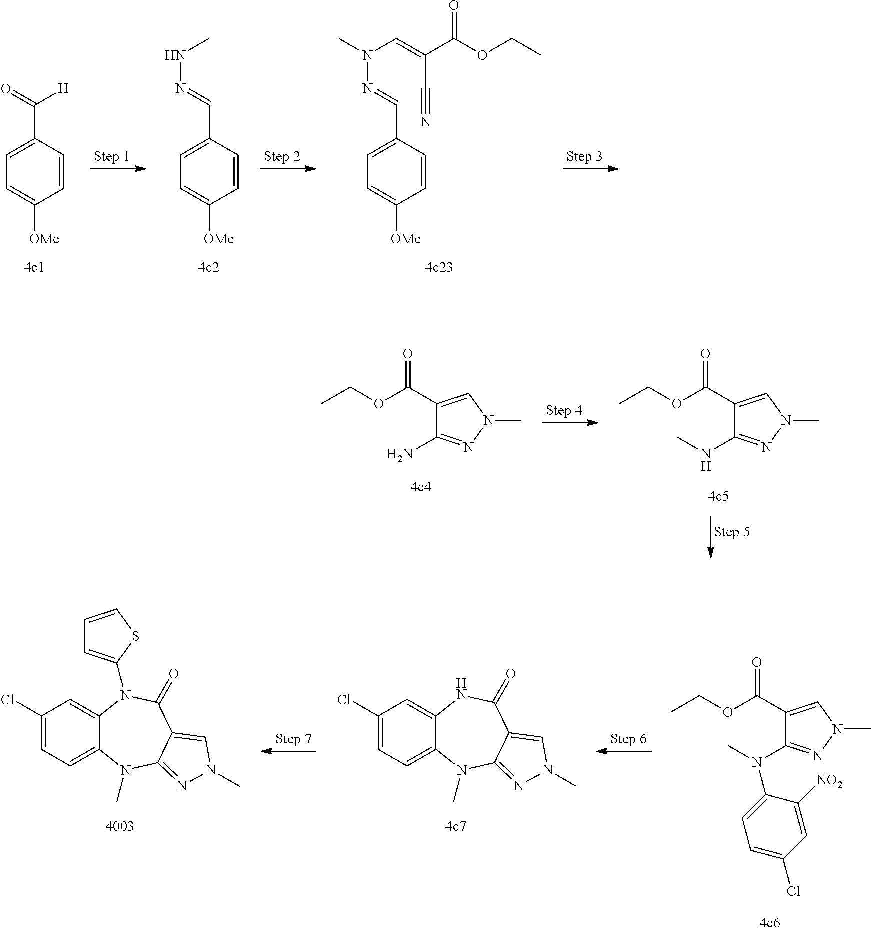

### Step 1

To a solution of methylhydrazine (Aldrich, 0.92 mL, 17.2 mmol) in EtOH (30 mL) is added 4c1 (Aldrich, 1.97 mL, 17.2 mmol). After 4 h at RT the reaction mixture is concentrated to dryness to give hydrazone 4c2.

### Step 2:

To a solution of 4c2 (2.8 g, 17.1 mmol) in benzene (3.5 mL) is added a solution of ethyl(ethoxymethylene)cyanoacetate (Aldrich, 2.9 mL, 17.1 mmol) in benzene (6 mL), and the reaction mixture is heated to reflux for 3 h. The resulting precipitate is recovered by filtration and the solid washed with benzene to give compound 4c3.

### Step 3:

To a suspension of 4c3 (4.7 g, 16.36 mmol) in boiling EtOH (16 mL) is added concentrated HCl solution (1.6 mL) dropwise. After 30 min, the reaction mixture is concentrated to dryness. The oily residue is triturated with boiling Et₂O (30 mL), the resulting suspension is filtered and the solid treated with aqueous 1 N NaOH solution (17 mL). The aqueous layer is extracted with CHCl₃ washed with brine, dried (MgSO₄), and concentrated under vacuum. The residue is triturated with hot Et₂O to give compound 4c4.

### Step 4:

To a solution of compound 4c4 (952 mg, 5.6 mmol) in THF (28 mL) is added NaH (60%, 270 mg, 6.75 mmol), After 1 h at RT, MeI (0.7 mL, 11.3 mmol) is added and the mixture is heated to 70° C. for 16 h. The reaction mixture is evaporated to dryness, diluted with DCM then washed with water and brine, dried (MgSO₄), and concentrated under vacuum. The residue is purified by flash chromatography (EtOAc:hexane, 1:1) to give compound 4c5.

### Step 5:

To a solution of 4c5 (184 mg, 1.0 mmol) in THF (10 mL) is added a 1.0 M NaHMDS solution in THF (2.0 mL, 2.0 mmol) followed by 2-fluoro-5-chloro-nitrobenzene (ABCR, 176.3 mg, 1 mmol). After 2 h at 70° C., the reaction mixture is evaporated to dryness then diluted with DCM, washed with water and brine, dried (MgSO₄), and concentrated under vacuum. The residue is purified by flash chromatography (EtOAc:hexane, 3:7) to give compound 4c6.

### Step 6:

A mixture of 4c6 (71 mg, 0.21 mmol) in AcOH (2 mL) and iron (116 mg, 2.1 mmol) is heated to 115° C. for 25 h. The reaction mixture is cooled to RT, diluted with CHCl₃, and filtered. The filtrate is concentrated under vacuum and the residue is purified by flash chromatography (EtOAc:hexane, 8:2) to give compound 4c7.

### Step 7:

A mixture of 4c7 (38.9 mg, 0.15 mmol), CuI (28 mg, 0.15 mmol), K₂CO₃ (24.6 mg, 0.18 mmol) and 2b1 (79 μL, 0.81 mmol) in DMF (0.4 mL) is heated to 140° C. for 48 h. The reaction mixture is cooled to RT and diluted with EtOAc, washed with water and brine, dried (MgSO₄), and concentrated under vacuum. The residue is purified by flash chromatography (EtOAc:hexane, 1:1) to give compound 4003.

### Example 4d

### Preparation of Compound 4004

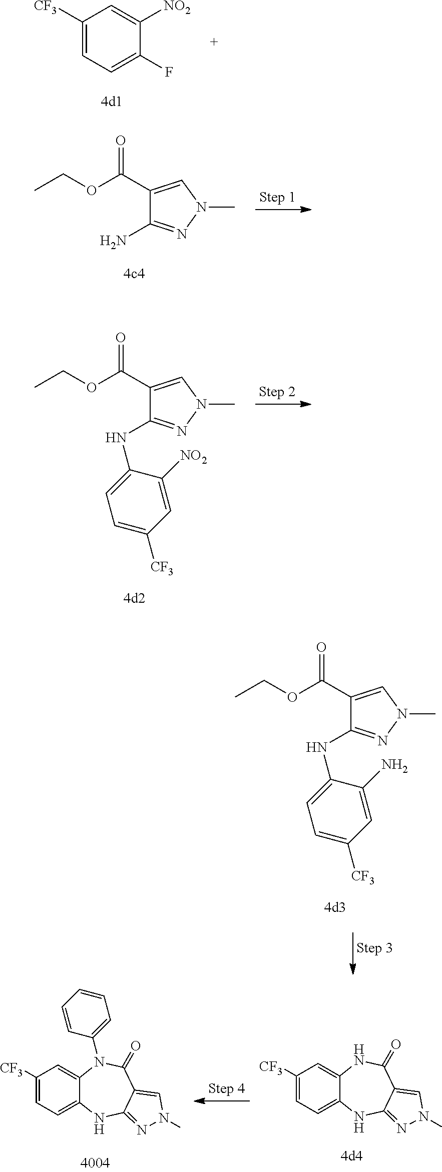 [SMILES: O=[N+]([O-])c1cc(C(F)(F)F)ccc1F ; CCOC(=O)c1cn(C)nc1N ; CCOC(=O)c1cn(C)nc1Nc1ccc(C(F)(F)F)cc1[N+](=O)[O-] ; CCOC(=O)c1cn(C)nc1Nc1ccc(C(F)(F)F)cc1N ; Cn1cc2c(n1)Nc1ccc(C(F)(F)F)cc1N(c1ccccc1)C2=O ; Cn1cc2c(n1)Nc1ccc(C(F)(F)F)cc1NC2=O]

### Step 1:

Following the procedure described in step 5 of example 4c, 4d1 (Aldrich) is transformed into 4d2.

### Step 2:

A mixture of 4d2 (1.50 g, 4.19 mmol) and 10% Pd/C (200 mg) in THF (30 mL) is stirred under a H₂ atmosphere for 18 h. The reaction mixture is filtered through Celite® and concentrated under vacuum to give compound 4d3.

### Step 3:

To a solution of NaH (60% in oil, 0.24 mg, 5.94 mmol) in DMSO (20 mL) at 65° C. is added dropwise a solution of 4d3 (1.30 g, 3.96 mmol) in DMSO (5.0 mL), after which stirring is continued for 1 h, The reaction mixture is poured into water, extracted with EtOAc. The organic layer is dried (MgSO₄) and concentrated under vacuum to give compound 4d4.

### Step 4:

Following a similar procedure as described in step 4 of example 2a, compound 4d4 is transformed into compound 4004.

### Example 4e

### Preparation of Compound 4013

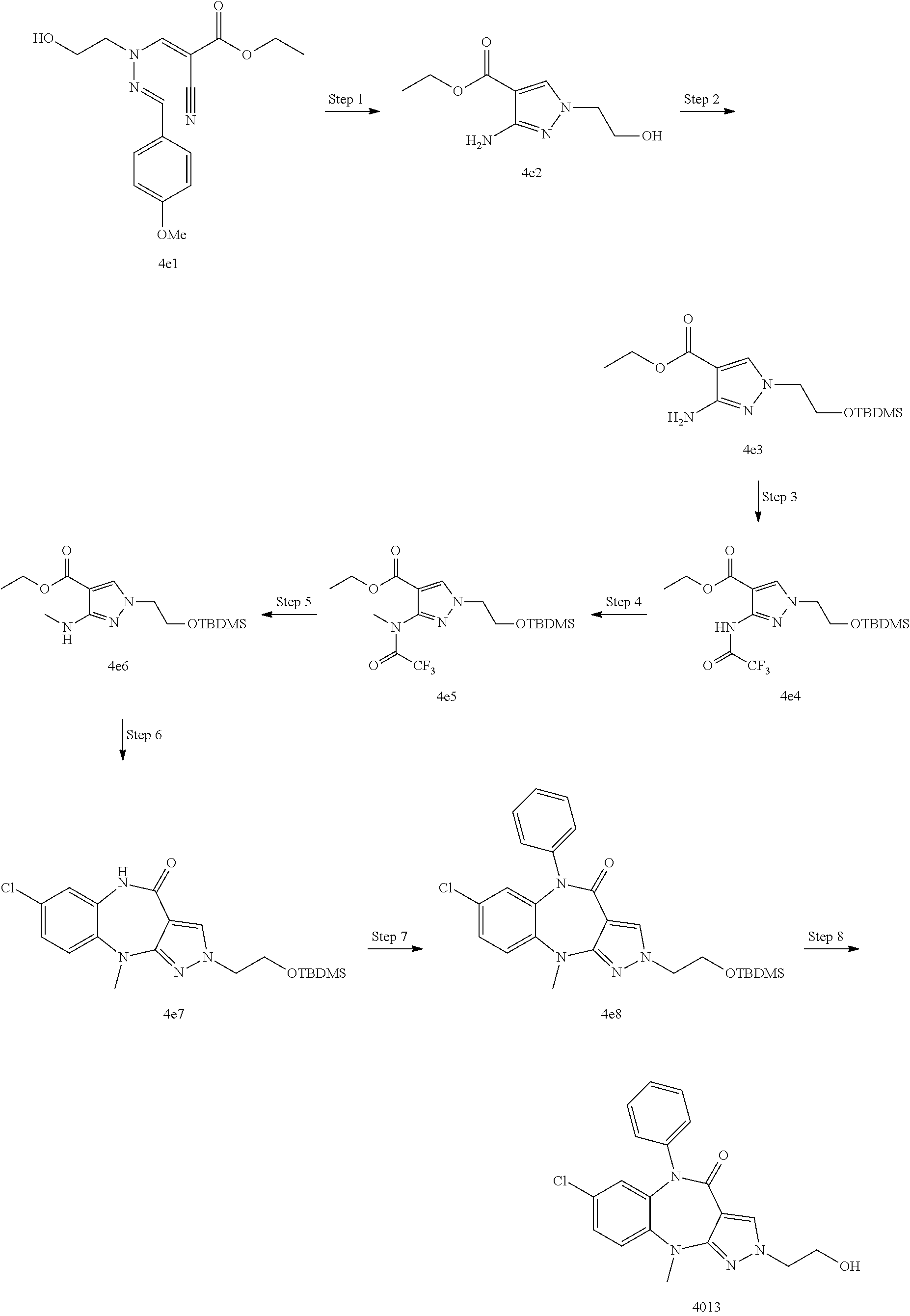

### Step 1:

Compound 4e1 is prepared from 2-hydroxyethylhydrazine as described in steps 1 and 2 of example 4c. Concentrated aqueous HCl solution is added dropwise to a warm solution of 4e1 (44.7 g, 141 mmol in EtOH (250 mL). The mixture is heated to reflux for 30 min. The cooled mixture is concentrated under reduced pressure. The residue is triturated four times with Et₂O (200 mL). The residue is dissolved in DCM (175 mL) and is stirred at RT for 15 min. The resulting suspension is filtered and the solid is washed with DCM, dried under reduced pressure to give 4e2.

### Step 2:

A solution of TBDMS-Cl (7.80 g, 51.7 mmol) in DMF (10 mL) is added to an ice-cold solution of 4e2 (9.74 g, 41.3 mmol) and imidazole (8.44 g, 124 mmol) in DMF (60 mL). The mixture is allowed to warm to RT and is stirred at RT for 16 h. The mixture is poured into water and extracted with EtOAc. The combined organic layers are washed with aqueous 0.25 N HCl solution, aqueous saturated NaHCO₃ and brine, dried (MgSO₄), filtered and concentrated under reduced pressure to give compound 4e3.

### Step 3:

Trifluoroacetic anhydride (5.40 mL, 38.2 mmol) is added to an ice-cold solution of 4e3 (10.0 g, 31.9 mmol) and pyridine (4.50 mL, 55.6 mmol) in DCM (80 mL). The reaction mixture is stirred at RT for 3 h. The mixture diluted with DCM is washed with aqueous 1 N HCl solution, aqueous saturated NaHCO₃ and brine, dried (MgSO₄), filtered and concentrated under reduced pressure to give compound 4e4.

### Step 4:

MeI (1.53 mL, 24.4 mmol) is added to a mixture of 4e4 (9.49 g, 23.2 mmol) and K₂CO₃ (3.52 g, 25.5 mmol) in DMF (20 mL). After 3 h, an additional amount of MeI (0.3 mL, 4.82 mmol) is added. The mixture is stirred at RT for 16 h. The mixture is poured into water and extracted with Et₂O. The combined organic layers are washed with water and brine, dried (MgSO₄), filtered and concentrated under reduced pressure to give compound 4e5.

### Step 5:

A solution of 21% EtONa in EtOH (9.60 mL, 28.8 mmol) is added to an ice-cold solution of 4e5 (9.45 g, 22.3 mmol) in EtOH (80 mL). The mixture is stirred at RT for 16 h then is poured into water. The mixture is extracted with EtOAc. The combined organic layers are washed with diluted brine, aqueous 5% NaHCO₃ solution and brine, dried (MgSO₄), filtered and concentrated under reduced pressure to give compound 4e6.

### Step 6:

To an ice-cold solution of 4e6 (6.06 g, 18.5 mmol) and 2,5-dichloronitrobenzene (Aldrich; 3.70 g, 19.3 mmol) in THF (15 mL) is added a 1.0 M NaHMDS solution in THF (37.0 mL, 37.0 mmol). After 2.5 h, AcOH (5 mL) is added and the mixture is concentrated under reduced pressure. The residue is dissolved in AcOH (50 mL) and iron powder (8.27 g, 148 mmol) is added and the mixture is heated to 105° C. for 3 h. The cooled mixture is poured into water and is extracted with Et₂O. The combined organic layers are washed with water, aqueous saturated NaHCO₃ solution and brine, dried (MgSO₄), filtered and concentrated under reduced pressure. The residue is purified by flash chromatography (hexane:EtOAc, 2:1 to 1:1) to give compound 4e7.

### Step 7:

Using the procedure described in step 4 of example 2a, compound 4e7 is transformed into compound 4e8.

### Step 8:

A solution of 1.0 M TBAF in THF (5.82 mL, 5.82 mmol) is added dropwise to a solution of 4e8 (2.34 g, 4.84 mmol) in THF (25 mL). The mixture is stirred at RT for 1 h. The mixture is poured into water and extracted with EtOAc. The combined organic extracts are washed with aqueous 0.5 M HCl solution, diluted brine and brine, dried (MgSO₄), filtered and concentrated under reduced pressure to give compound 4013.

### Example 4f

### Preparation of Compound 4011

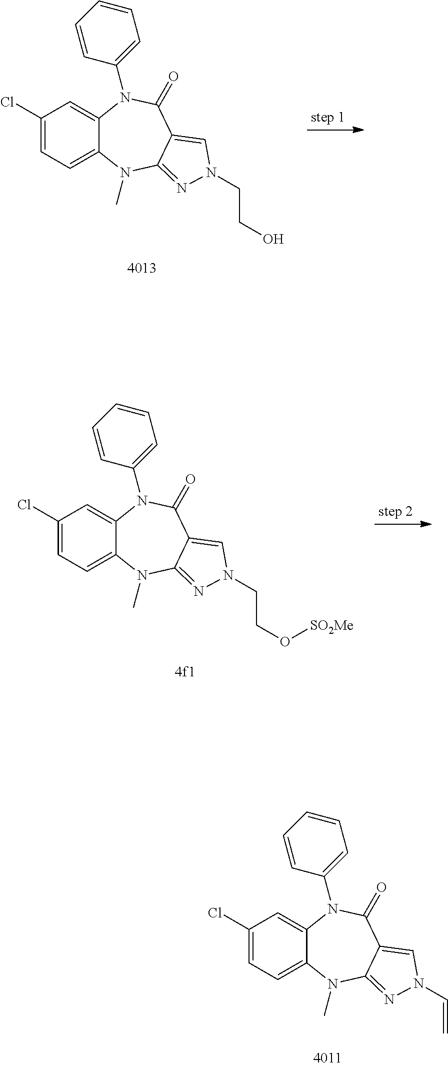 [SMILES: CN1c2ccc(Cl)cc2N(c2ccccc2)C(=O)c2cn(CCO)nc21 ; CN1c2ccc(Cl)cc2N(c2ccccc2)C(=O)c2cn(CCOS(C)(=O)=O)nc21 ; C=Cn1cc2c(n1)N(C)c1ccc(Cl)cc1N(c1ccccc1)C2=O]

### Step 1:

MsCl (21 μL, 0.27 mmol) is added to a solution of 4013 (50 mg, 0.14 mmol) and pyridine (49 μL, 0.61 mmol) in DCM (0.5 mL). The reaction mixture is stirred at RT for 4 h. The mixture is poured in water and extracted with EtOAc. The organic layers are washed with aqueous 0.2 M HCl solution, aqueous saturated NaHCO₃ solution and brine, dried (MgSO₄), filtered and concentrated under reduced pressure to give compound 4f1.

### Step 2:

A mixture of 4f1 (12 mg, 27 mmol) and DBU (8.1 μL, 54 μmol) in DMF (0.5 mL) is stirred at RT for 0.5 h then heated briefly to 100° C. for 1 min and maintained at 75° C. for 2 h. The crude mixture is purified by preparative HPLC to give compound 4011.

### Example 4g

### Preparation of Compound 4020

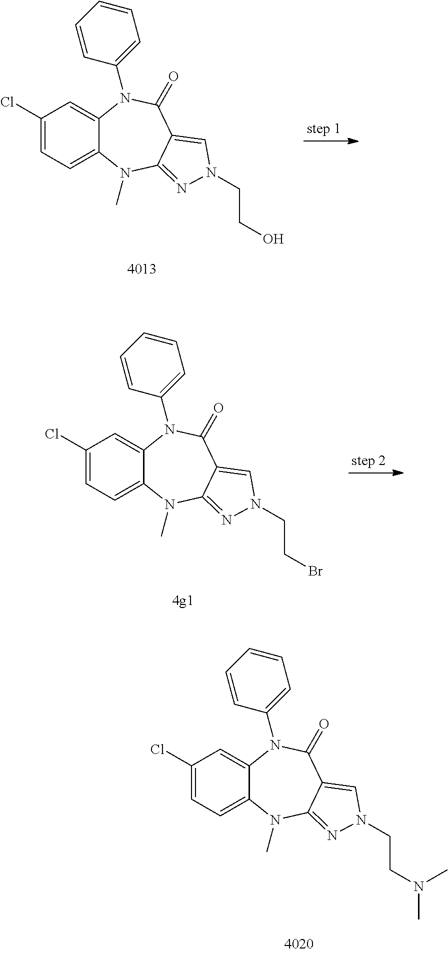 [SMILES: CN1c2ccc(Cl)cc2N(c2ccccc2)C(=O)c2cn(CCO)nc21 ; CN1c2ccc(Cl)cc2N(c2ccccc2)C(=O)c2cn(CCBr)nc21 ; CN(C)CCn1cc2c(n1)N(C)c1ccc(Cl)cc1N(c1ccccc1)C2=O]

### Step 1:

A solution of 4013 (550 mg, 1.49 mmol), CBr₄ (966 mg, 2.91 mmol), Ph₃P (508 mg, 1.94 mmol) in DCM (5.0 mL) is stirred at RT for 3 h. The mixture is concentrated under reduced pressure and the residue purified by flash chromatography (hexane:EtOAc) to give compound 4g1.

### Step 2:

A solution of 4g1 (30.0 mg, 69.5 Et₃N (29.1 μL, 208 μmol) and a 2.0 M Me₂NH solution in THF (70.0 μL, 139 μmol) is heated to 70° C. for 1.5 h. The cooled mixture is diluted with AcOH (0.5 mL) and DMSO (1.0 mL) and purified by preparative HPLC to give compound 4020.

### Example 4h

### Preparation of Compounds 4014 and 4019

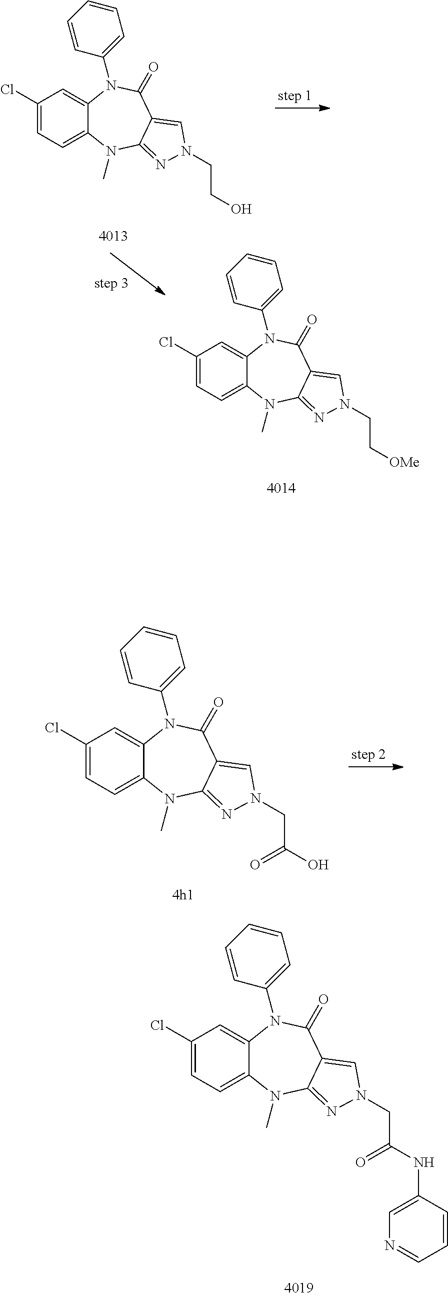

### Step 1:

Dess-Martin periodinane (575 mg, 1.36 mmol) is added to a solution of 4013 (200 mg, 0.542 mmol) in wet DCM (4.0 mL). The mixture is stirred at RT for 2 days. The reaction mixture is concentrated under reduced pressure to give compound 4h1, which is used without further purification.

### Step 2:

3-Aminopyridine (18.4 mg, 196 μmol) is added to a solution of 4h1 (25.0 mg, 65.3 mmol), N-methylmorpholine (28.7 μL, 261 μmol) and TBTU (35.6 mg, 114 mmol) in DMSO (0.5 mL). The mixture is stirred at RT for 16 h. The mixture is purified by preparative HPLC to give compound 4019.

### Step 3:

NaH (3.60 mg, 142 μmol) is added to an ice-cold solution of 4013 (35.0 mg, 94.9 μmol) and MeI (11.8 μL, 190 μmol) in THF (0.5 mL). The mixture is stirred at 0° C. for 15 min then at RT for 30 min. The mixture is concentrated under reduced pressure, diluted with DMSO (1 mL) and AcOH (0.5 mL) and purified by preparative HPLC to give compound 4014.

### Example 41

### Preparation of Compounds 4007, 4012, 4025 and 4029

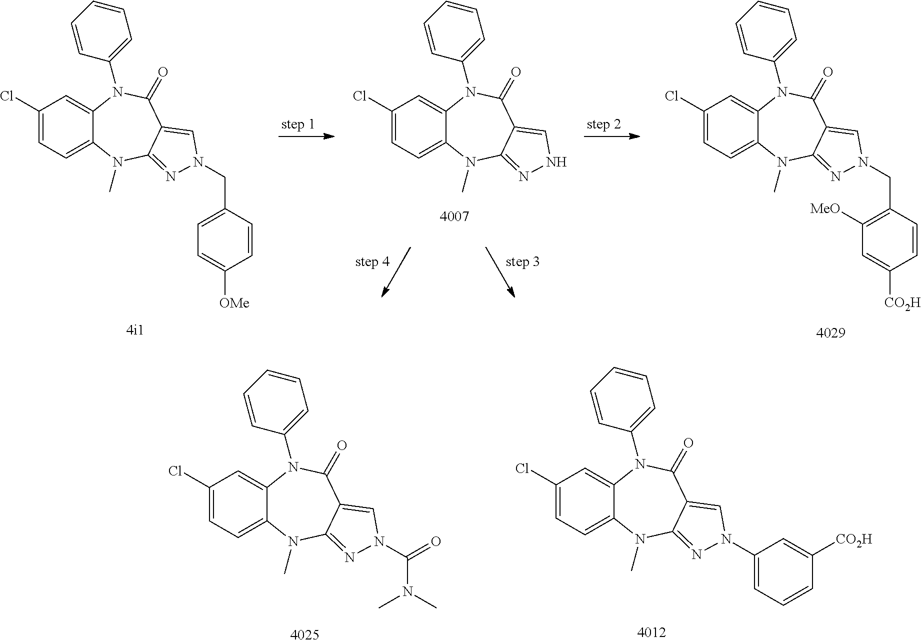 [SMILES: COc1ccc(Cn2cc3c(n2)N(C)c2ccc(Cl)cc2N(c2ccccc2)C3=O)cc1 ; CN1c2ccc(Cl)cc2N(c2ccccc2)C(=O)c2c[nH]nc21 ; COc1cc(C(=O)O)ccc1Cn1cc2c(n1)N(C)c1ccc(Cl)cc1N(c1ccccc1)C2=O ; CN(C)C(=O)n1cc2c(n1)N(C)c1ccc(Cl)cc1N(c1ccccc1)C2=O ; CN1c2ccc(Cl)cc2N(c2ccccc2)C(=O)c2cn(-c3cccc(C(=O)O)c3)nc21]

### Step 1:

Compound 4i1 is prepared from 4-methoxybenzylhydrazine as described in steps 1 and 2 of example 4c and steps 1-7 of example 4e. A solution of 4i1 (1.75 g, 3.93 mmol) in TFA (60 mL) is heated to reflux for 25 h. The mixture is concentrated under reduced pressure and the residue extracted with DCM. The combined extracts are washed with saturated aqueous NaHCO₃ solution and brine, dried (MgSO₄), filtered and concentrated under reduced pressure. The residue is purified by flash chromatography (EtOAc:hexane, 3:1) to give compound 4007.

### Step 2:

A solution of 1.0 M NaHMDS in THF (75 μL, 75 μmol) is added dropwise to an ice-cold solution of 4007 (25.0 mg, 77.0 mmol) in THF (0.5 mL). After the addition, the mixture is allowed to warm to RT and DMF (0.1 mL) is added. A solution of methyl (4-bromomethyl)-3-methoxybenzoate (Aldrich; 26.0 mg, 100 μmol) in THF (0.2 mL) is added and the mixture is stirred at RT for 1.5 h. An aqueous 1.0 N LiOH solution (0.25 mL, 0.25 mmol) and MeOH (0.15 mL) are added and the mixture is stirred at RT for 16 h. The mixture is acidified by addition of AcOH and the crude mixture is purified by preparative HPLC to give compound 4029.

### Step 3:

A mixture of 4007 (30.0 mg, 92.4 μmol), (3-carboxyphenyl)boronic acid (CombiBlocks, 30.6 mg, 185 μmol), Cu(OAc)₂ (33.6 mg, 185 μmol) and pyridine (22.4 μL, 277 μmol) in DMF (0.3 mL) is stirred at RT for 1 h and then is heated to 85° C. for 1 h. The cooled mixture is filtered, diluted with DMSO and purified by preparative HPLC to give compound 4012.

### Step 4:

Dimethylcarbamoyl chloride (17.0 μL, 185 μmol) is added to a solution of 4007 (30.0 mg, 92.4 μmol) and DBU (30.0 μL, 201 μmol) in THF (0.4 mL). The mixture is stirred at RT for 2 h. Water and DMSO are added and the mixture purified by preparative HPLC to give compound 4025.

### Example 4j

### Preparation of Compound 4010

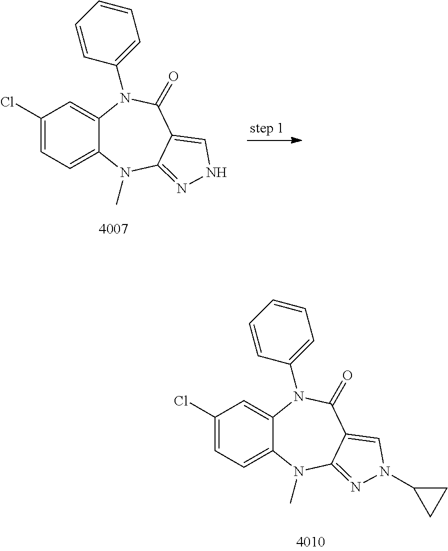 [SMILES: CN1c2ccc(Cl)cc2N(c2ccccc2)C(=O)c2c[nH]nc21 ; CN1c2ccc(Cl)cc2N(c2ccccc2)C(=O)c2cn(C3CC3)nc21]

### Step 1:

A mixture of 4007 (30.0 mg, 92.4 μmol), c-Pr₃Bi (124 mg, 373 μmol) (prepared using a literature procedure: Gagnon, A.; St-Onge, M.; Little, K.; Duplessis, M.; Barabé, F. J. Am. Chem. Soc. 2007, 129, 44-45), Cu(OAc)₂ (68.0 mg, 374 μmol) and pyridine (30.0 μL, 371 μmol) in 1,4-dioxane (1.0 mL) is heated to 60° C. for 2 days. The cooled mixture is filtered, diluted with DMSO and purified by preparative HPLC to give compound 4010.

### Example 5a

### Preparation of Compounds 5007, 5008 and 5020

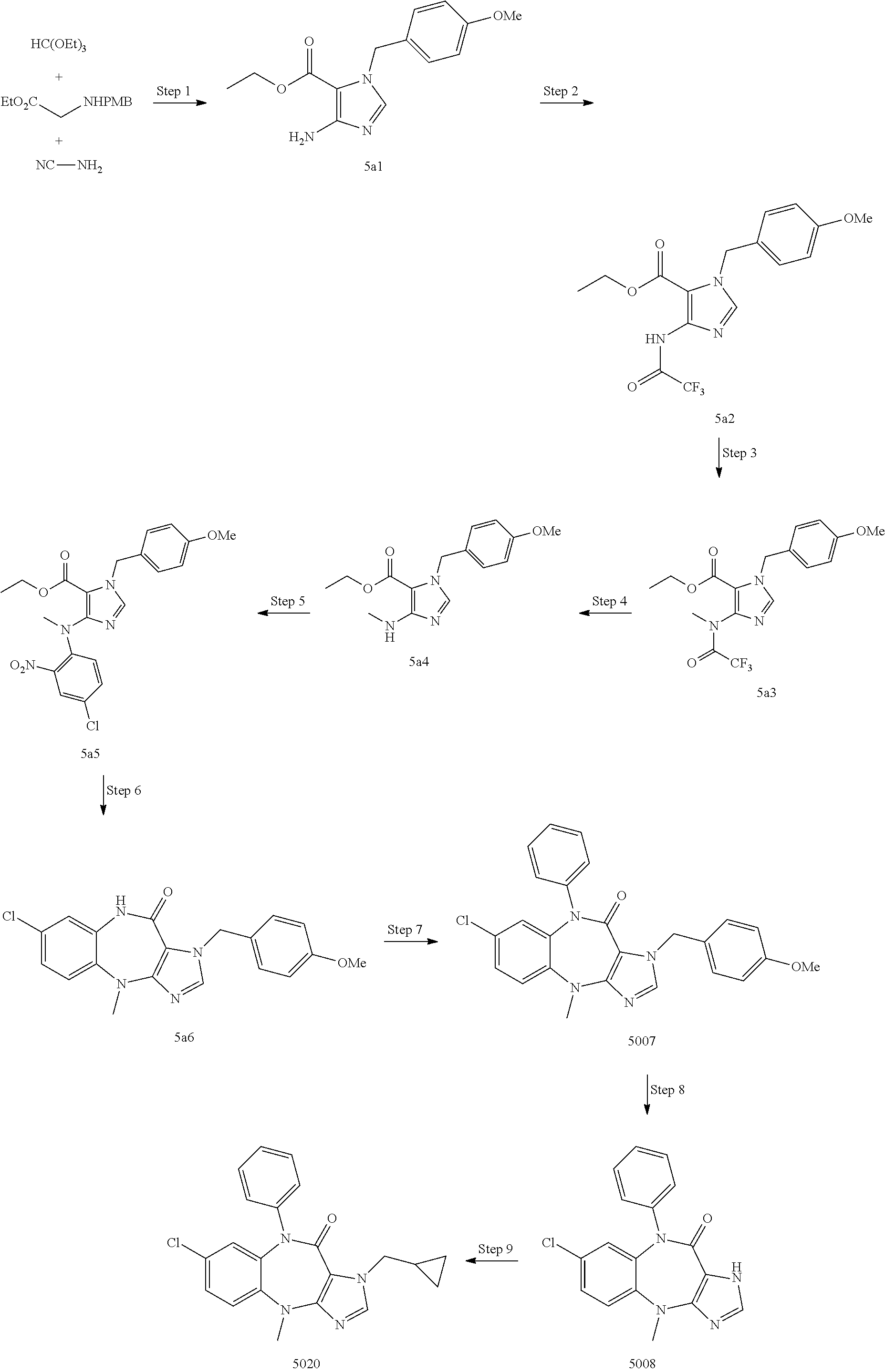

### Step 1:

HC(OEt)₃ (100 mL, 620 mmol) and cyanamide (14 g, 330 mmol) are heated to reflux for 2 h. Heating is removed until reflux subsides and then a Dean-Stark trap is attached to the flask and reflux is continued. After about 5 mL of EtOH is collected, the reaction is cooled to RT and the mixture is concentrated to a mass of about 68 g. From this, about 9.3 g is dissolved in Et₂O and N-(p-methoxybenzyl)glycine ethyl ester (12 g, 55 mmol) is added and the mixture is stirred for 1 h at RT. The mixture is concentrated and redissolved in a solution of 21% NaOEt in EtOH (18 mL, 55 mmol). After 1 h of stirring at RT, the mixture is cooled to 4° C. and is allowed to stand at this temperature for 12 h. The solid that forms is removed by filtration and the filtrate is evaporated to dryness to provide compound 5a1.

### Step 2:

Trifluoroacetic anhydride (4.0 mL, 30 mmol) is added to a solution of compound 5a1 (6.0 g, 20 mmol) and pyridine (3.1 mL, 39 mmol) in DCM (100 mL) at 0° C. The reaction mixture is then warmed to RT and is stirred for 2 h. The reaction mixture diluted with DCM is washed with water, aqueous saturated NaHCO₃ solution, dried over MgSO₄ and concentrated to give compound 5a2.

### Step 3:

MeI (0.35 mL, 5.5 mmol) is added to a suspension of 5a2 (1.9 g, 5.0 mmol) and K₂CO₃ (0.86 g, 6.3 mmol) in dry DMF (10 mL) under a N₂ atmosphere and the mixture is stirred vigorously for 1 h. The mixture is diluted with water, extracted with EtOAc and the organic layer is washed with water. The organic layer is dried over MgSO₄, evaporated to dryness and the residue is purified by flash chromatography (EtOAc:hexane, 1:1) to give compound 5a3.

### Step 4:

A solution of 21% NaOEt in EtOH (1.5 mL, 4.6 mmol) is added to a solution of 5a3 in EtOH (12 mL) and the reaction mixture is stirred at RT for 1 h. The mixture is diluted with EtOAc and washed with water/brine (1:1). The organic layer is dried over MgSO₄, evaporated to dryness and the residue is purified by flash chromatography (acetone:CHCl₃, 1:4) to give compound 5a4.

### Step 5:

A 1.0 M NaHMDS solution in THF (3.7 mL, 3.7 mmol) is added dropwise to a solution of 5a4 (830 mg, 2.9 mmol) and 5-chloro-2-fluoronitrobenzene (Apollo, 560 mg, 3.2 mmol) in THF (10 mL) with stirring under a N₂ atmosphere for 2 h. The reaction mixture is diluted with H₂O and Et₂O. The phases are separated and the aqueous layer is extracted with Et₂O. The combined organic layers are dried over MgSO₄ and concentrated to dryness to give compound 5a5.

### Step 6:

A mixture of 5a5 (1.4 g, 3.1 mmol), Fe powder (860 mg, 15 mmol) and AcOH (18 mL) is heated to 100° C. and stirred vigorously for 2 h. The reaction is cooled to RT, diluted with PhMe and evaporated to dryness. The residue is partitioned between water and EtOAc and the water layer is extracted with EtOAc. The combined organic layers are dried over MgSO₄ and concentrated to dryness. The product is purified by flash chromatography (EtOAc:hexane, 6:4) to give compound 5a6.

### Step 7:

Compound 5007 is prepared from 5a6 using the procedure described in step 4 of example 1a for the preparation of 1a5.

### Step 8:

Neat TfOH (420 μL, 2.8 mmol) is added to a solution of 5007 (440 mg, 0.99 mmol) dissolved in TFA (20 mL) and the mixture is stirred at RT for 3.5 h. Water (5 mL) is added and the mixture is concentrated nearly to dryness. The residue is diluted with aqueous saturated NaHCO₃ solution and extracted with EtOAc. The combined organic layers are dried over Na₂SO₄ and concentrated to dryness. The product is purified by flash chromatography (EtOAc:hexane, 4:1) to give compound 5008.

### Step 9:

A solution of 1.0 M NaHMDS in THF (50 μL, 0.05 mmol) is added to a solution of 5008 dissolved in THF (1 mL) and the mixture is stirred at RT for 5 min. DMF (500 μL) followed by (bromomethyl)cyclopropane (35 mg, 0.25 mmol) are added and the reaction mixture is stirred for 5 h. Water (3 mL) is added and the reaction mixture is concentrated. The residue is purified by flash chromatography (hexane:EtOAc, 3:7) to give compound 5020.

### Example 5b

### Preparation of Compound 5026

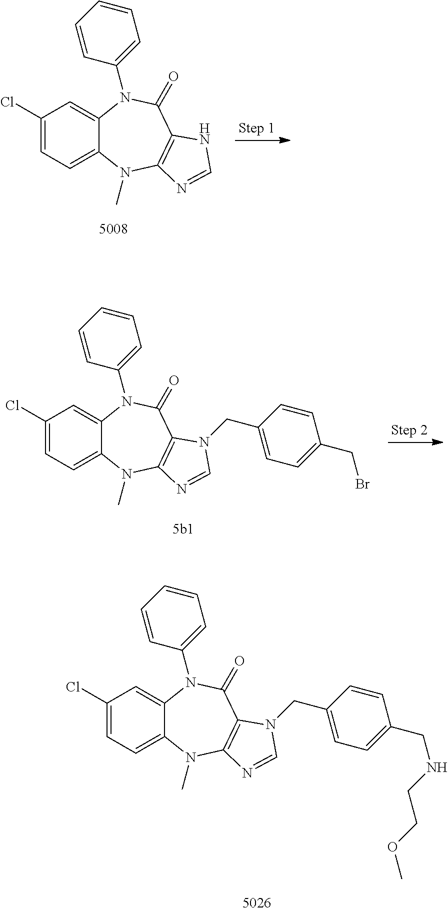 [SMILES: CN1c2ccc(Cl)cc2N(c2ccccc2)C(=O)c2[nH]cnc21 ; CN1c2ccc(Cl)cc2N(c2ccccc2)C(=O)c2c1ncn2Cc1ccc(CBr)cc1 ; COCCNCc1ccc(Cn2cnc3c2C(=O)N(c2ccccc2)c2cc(Cl)ccc2N3C)cc1]

### Step 1:

Compound 5b1 is prepared from 5008 analogously to the procedure described for compound 5020 in step 9 of example 5a except that α,α′-dibromo-p-xylene (Aldrich) is used in place of (bromomethyl)cyclopropane.

### Step 2:

A solution of 5b1 (20 mg, 0.04 mmol) in DMF (2 mL) is added to a solution of 2-methoxyethylamine (40 mg, 0.6 mmol) in a solution of DMF (100 μL). The reaction mixture is stirred for 16 h then water (5 mL) is added and the mixture is concentrated under reduced pressure. The residue is purified by preparative HPLC to give compound 5026.

### Example 5c

### Preparation of Compound 5025

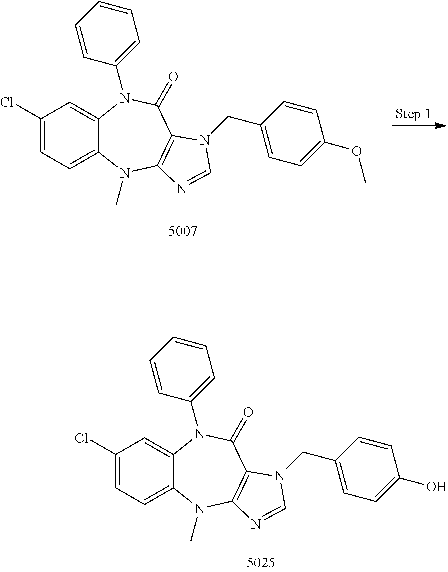 [SMILES: COc1ccc(Cn2cnc3c2C(=O)N(c2ccccc2)c2cc(Cl)ccc2N3C)cc1 ; CN1c2ccc(Cl)cc2N(c2ccccc2)C(=O)c2c1ncn2Cc1ccc(O)cc1]

### Step 1:

A solution of 1.0 M BBr₃ in DCM (360 μL, 0.36 mmol) is added to a solution of 5007 (65 mg, 0.14 mmol) in DCM (1 mL) under a N₂ atmosphere and the mixture is stirred for 30 min. Water (1 mL) and aqueous saturated NaHCO₃ solution (1 mL) are added and the mixture is extracted with DCM. The combined organic layers are dried over Na₂SO₄ and concentrated. The residue is purified by preparative HPLC to give compound 5025.

### Example 5d

### Preparation of Compound 5009

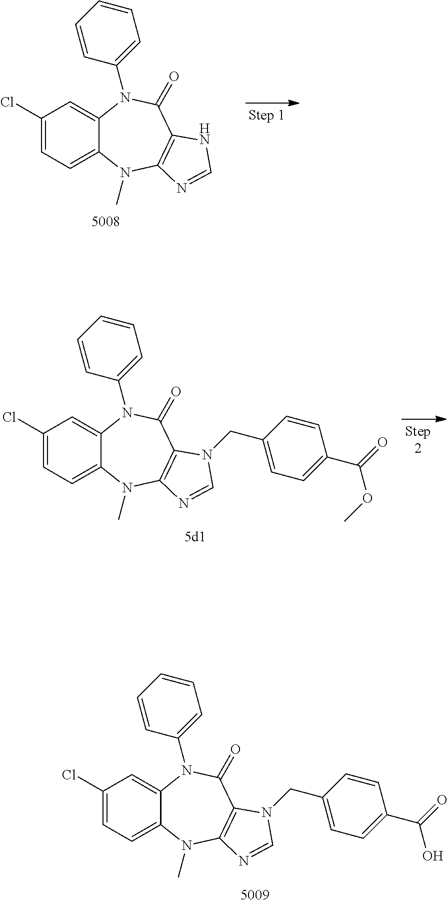 [SMILES: CN1c2ccc(Cl)cc2N(c2ccccc2)C(=O)c2[nH]cnc21 ; COC(=O)c1ccc(Cn2cnc3c2C(=O)N(c2ccccc2)c2cc(Cl)ccc2N3C)cc1 ; CN1c2ccc(Cl)cc2N(c2ccccc2)C(=O)c2c1ncn2Cc1ccc(C(=O)O)cc1]

### Step 1:

Compound 5d1 is prepared from 5008 analogously to the procedure described for compound 5020 in step 9 of example 5a except that 4-(bromomethyl)benzoic acid methyl ester (Aldrich) is used in place of (bromomethyl)cyclopropane.

### Step 2:

Aa aqueous 1.0 N LiOH solution (0.6 mL, 0.6 mmol) is added to a solution of 5d1 in THF (1.5 mL) and MeOH (1.5 mL) and the mixture is stirred at RT for 2 h. The reaction mixture is diluted with water, acidified with aqueous 1 N HCl solution and extracted with EtOAc. The organic layer is dried over MgSO₄ and the residue is purified by preparative HPLC to give compound 5009.

### Example 5e

### Preparation of Compound 5023

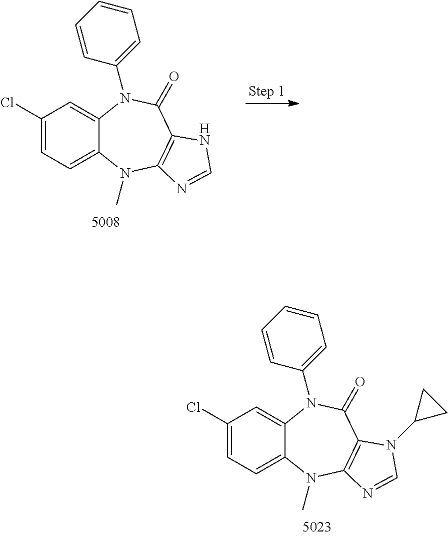 [SMILES: CN1c2ccc(Cl)cc2N(c2ccccc2)C(=O)c2[nH]cnc21 ; CN1c2ccc(Cl)cc2N(c2ccccc2)C(=O)c2c1ncn2C1CC1]

### Step 1:

Compound 5023 is prepared from compound 5008 analogously to the procedure described in step 4 of example 1a for the preparation of 1a5, except that c-Pr₃Bi (prepared using a literature procedure: Gagnon, A.; St-Onge, M.; Little, K.; Duplessis, M.; Barabé, F. J. Am. Chem. Soc. 2007, 129, 44-45) is used in place of Ph₃Bi and the reaction is stirred at 60° C. for 3 d.

### Example 5f

### Preparation of Compound 5021

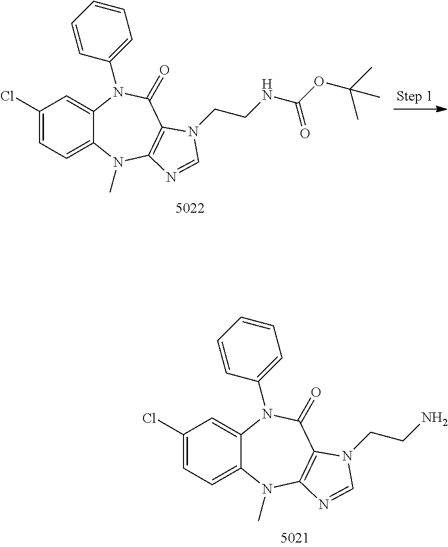 [SMILES: CN1c2ccc(Cl)cc2N(c2ccccc2)C(=O)c2c1ncn2CCNC(=O)OC(C)(C)C ; CN1c2ccc(Cl)cc2N(c2ccccc2)C(=O)c2c1ncn2CCN]

### Step 1:

Compound 5022 is prepared from compound 5008 using the procedure described in step 9 of example 5a for the preparation of 5020, except that N-(2-iodoethyl)-tert-butylcarbamate is used in place of (bromomethyl)cyclopropane. Compound 5022 (20 mg, 0.04 mmol) is dissolved in DCM (500 μL) and TFA (500 μL). The mixture is stirred at RT for 15 min and then concentrated. The residue is purified by preparative HPLC to give compound 5021.

### Example 6a

### Preparation of Compound 6002

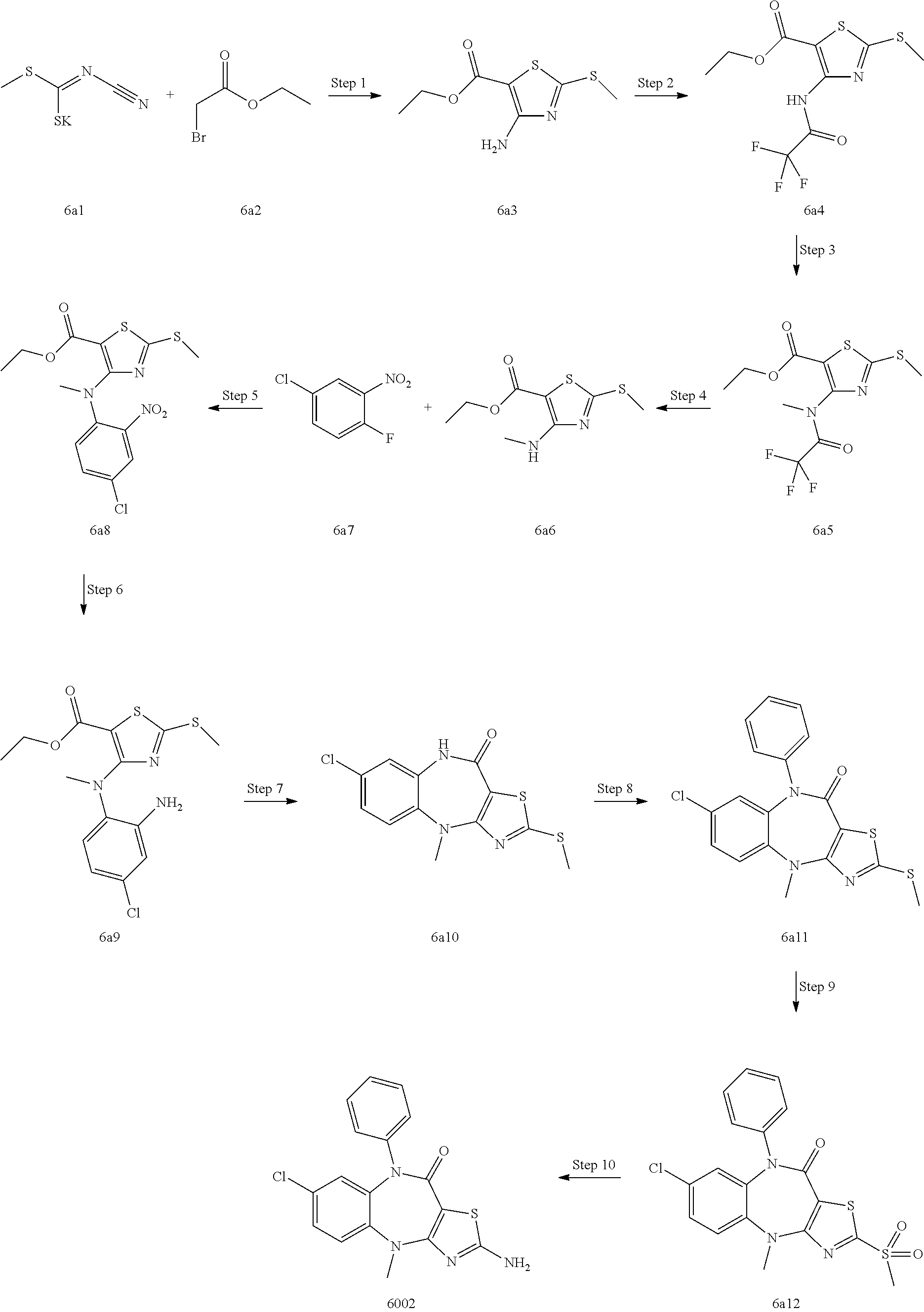

### Step 1:

To a stirred solution of 6a1 (Acros; 5.0 g, 29.4 mmol) in EtOH (50 mL) is added 6a2 (Aldrich, 3.3 mL, 29.4 mmol). The mixture is heated to reflux for 30 min. After cooling, Et₃N (2.14 mL, 29.4 mmol) is added and the mixture is heated to reflux for 2 h. The ethanol is evaporated and the residue triturated with water (150 mL). before being filtered and dried. The solid recovered by filtration is dried and then recrystallized from acetone to give compound 6a3.

### Step 2:

To an ice-cold solution of 6a3 (3.43 g, 15.7 mmol) in DCM (50 mL) under a N₂ atmosphere is added pyridine (1.91 mL, 23.6 mmol), followed by the dropwise addition of trifluoroacetic anhydride (2.66 mL, 18.8 mmol). After 15 min, the solution is warmed to RT and stirred for 1 h. The crude reaction mixture is washed with an aqueous 1 N HCl solution and then an aqueous 5% NaHCO₃ solution before being dried (MgSO₄), filtered and concentrated to give compound 6a4.

### Step 3:

To a stirred ice-cold solution of 6a4 (4.95 g, 15.7 mmol) in THF (75 mL) is added Ph₃P (6.2 g, 23.6 mmol), MeOH (2 mL) and in small portions, solid azodicarboxylic dipiperidide (6.0 g, 23.7 mmol). The mixture is stirred for 15 min at 0° C., then is allowed to warm to RT and is stirred for 3 h. The mixture is diluted with Et₂O (250 mL) and the suspension is filtered. The filtrate is partially concentrated, stored at 5° C. overnight and again filtered to remove solids. The filtrate is evaporated and the residue purified by flash chromatography (EtOAc:hexane) to give compound 6a5.

### Step 4:

To a solution of 6a5 (870 mg, 2.65 mmol) in EtOH (25 mL) is added solid K₂CO₃ (550 mg, 4.0 mmol). The mixture is heated to reflux for 1 h. The solvent is evaporated and the residue partitioned between EtOAc (20 mL) and water (40 mL). The organic phase is dried (MgSO₄), filtered and concentrated to give compound 6a6.

### Step 5:

To an ice-cold solution of compound 6a6 (1.0 g, 4.3 mmol) and 6a7 (Apollo, 1.51 g, 8.6 mmol) in THF (20 mL) under a N₂ atmosphere is added dropwise a 1.0 M NaHMDS solution in THF (8.6 mL, 8.6 mmol). The mixture is then allowed to warm to RT and is stirred for an additional 1 h. The mixture is then cooled to 0° C. before the dropwise addition of aqueous 0.5 N KHSO₄ solution (2 mL). The THF is removed under reduced pressure and the residue diluted with EtOAc. The solution is washed with aqueous 0.5 N KHSO₄ solution, dried (MgSO₄), filtered and concentrated. The residue is purified by flash chromatography (EtOAc/hexane) to afford compound 6a8.

### Step 6:

To 6a8 (200 mg, 0.52 mmol) in i-PrOH (25 mL) is added 10% Pd/C (200 mg) before the system is purged with hydrogen gas and stirred at RT for 6 h. The system is evacuated and degassed before the catalyst is removed by filtration. The filtrate is concentrated and dried to give compound 6a9.

### Step 7:

To an ice-cold solution of compound 6a9 (240 mg, 0.67 mmol) in pyridine (4 mL) is added portionwise NaH (60% in oil, 54 mg, 1.34 mmol). The mixture is allowed to warm to RT and stirred for 1.5 h before being heated to 50° C. for 1.5 h. The mixture is cooled to RT, diluted with EtOAc and the solution washed with water and aqueous 1 N HCl solution, dried (MgSO₄), filtered and concentrated to give compound 6a10.

### Step 8:

A mixture of 6a10 (211 mg, 0.68 mmol), Cu(OAc)₂ (185 mg, 1.02 mmol), Ph₃Bi (449 mg, 1.02 mmol) and Et₃N (189 μL, 1.36 mmol) in DCM (2 mL) is heated to reflux for 1 h. The solvent is removed under reduced pressure and the residue purified by flash chromatography (EtOAc:hexane) to give compound 6a11.

### Step 9:

A solution of 6a11 (37 mg, 0.095 mmol) in CHCl₃ (0.5 mL) and m-CPBA (41 mg, 238.5 mmol) is heated to reflux for 45 min. The mixture is cooled and diluted with CHCl₃ before being washed with aqueous 5% NaHCO₃ solution. The organic phase is dried (MgSO₄), filtered and concentrated to give compound 6a12.

### Step 10:

Compound 6a12 (45 mg, 0.11 mmol) is treated with a 2.0 M ammonia solution in i-PrOH (500 μL, 1.0 mmol) and is then heated in a sealed tube at 50° C. for 30 min. The cooled solution is concentrated and purified by preparative HPLC to give compound 6002.

### Example 6b

### Preparation of Compound 6001

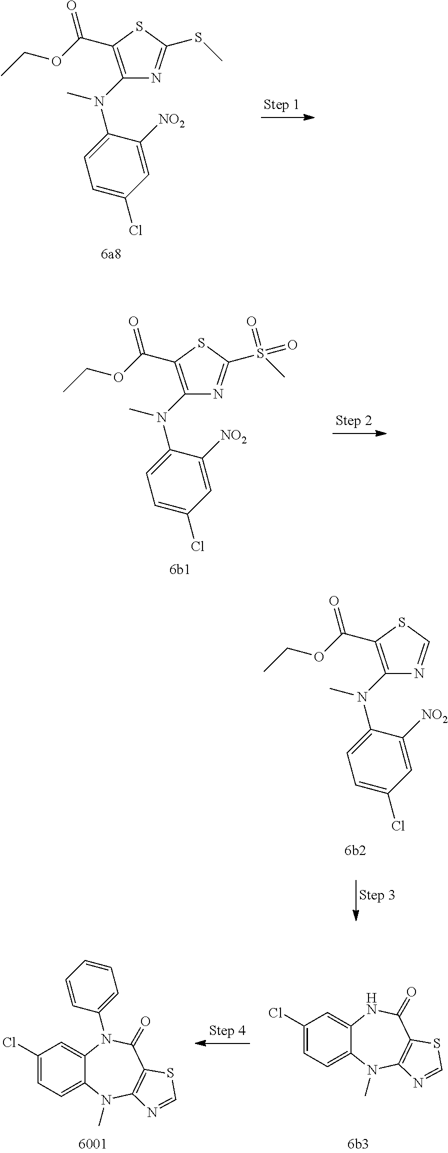 [SMILES: CCOC(=O)c1sc(SC)nc1N(C)c1ccc(Cl)cc1[N+](=O)[O-] ; CCOC(=O)c1sc(S(C)(=O)=O)nc1N(C)c1ccc(Cl)cc1[N+](=O)[O-] ; CCOC(=O)c1scnc1N(C)c1ccc(Cl)cc1[N+](=O)[O-] ; CN1c2ccc(Cl)cc2N(c2ccccc2)C(=O)c2scnc21 ; CN1c2ccc(Cl)cc2NC(=O)c2scnc21]

### Step 1:

A solution of 6a8 (300 mg, 0.77 mmol) and m-CPBA (391 mg, 1.93 mmol) in CHCl₃ (5 mL) is heated to reflux for 15 min. After cooling, the mixture is diluted with CHCl₃ and the solution is washed with an aqueous 5% NaHCO₃ solution and brine, dried (MgSO₄), filtered and concentrated to give compound 6b1.

### Step 2:

A solution of 6b1 (116 mg, 0.28 mmol) in EtOH (1 mL) is treated with NaBH₄ (10.6 mg, 0.28 mmol) in small portions. After 30 min, aqueous 1 N HCl solution (1.0 mL) is added and the mixture diluted with EtOAc. The phases are separated and the organic layer is washed with brine, dried (MgSO₄), filtered and concentrated to give compound 6b2.

### Step 3:

To compound 6b2 (115 mg, 0.34 mmol) in AcOH (1.0 mL) is added iron powder (50 mg) and the mixture is heated at 100° C. for 1.5 h. The cooled reaction mixture is diluted with water and EtOAc and the organic phase washed with aqueous 5% NaHCO₃ solution and brine, dried (MgSO₄), filtered and concentrated to give compound 6b3.

### Step 4:

Using the procedure described in step 8 of example 6a, 6b3 is transformed into compound 6001.

### Example 6c

### Preparation of Compound 6004

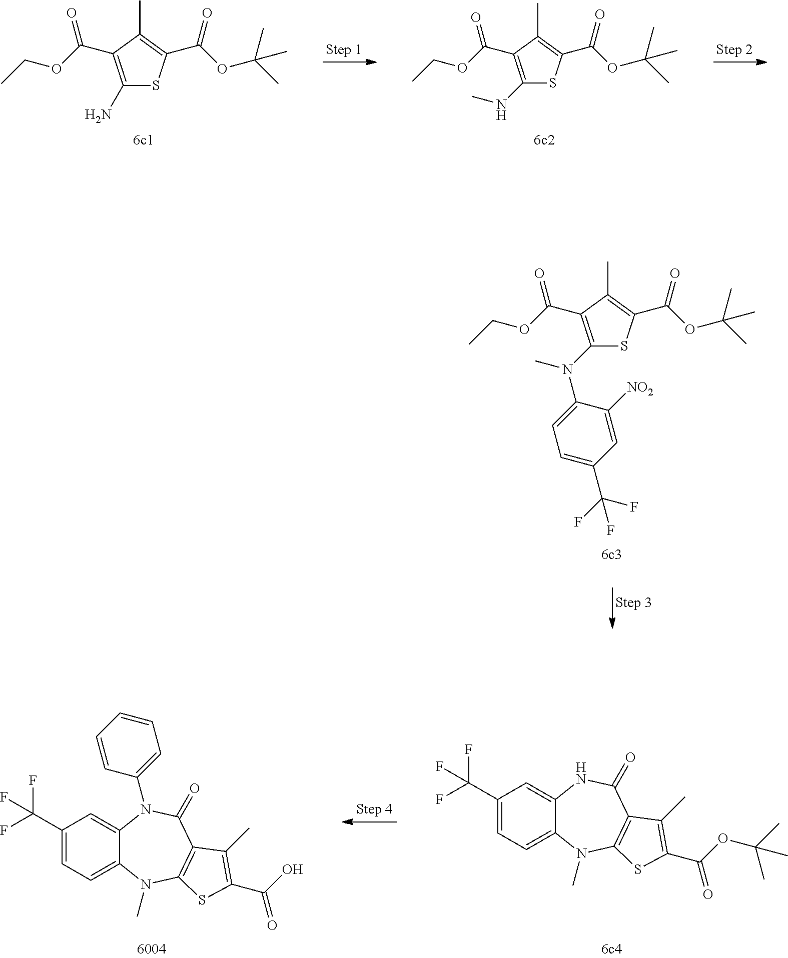 [SMILES: CCOC(=O)c1c(N)sc(C(=O)OC(C)(C)C)c1C ; CCOC(=O)c1c(NC)sc(C(=O)OC(C)(C)C)c1C ; CCOC(=O)c1c(N(C)c2ccc(C(F)(F)F)cc2[N+](=O)[O-])sc(C(=O)OC(C)(C)C)c1C ; Cc1c(C(=O)O)sc2c1C(=O)N(c1ccccc1)c1cc(C(F)(F)F)ccc1N2C ; Cc1c(C(=O)OC(C)(C)C)sc2c1C(=O)Nc1cc(C(F)(F)F)ccc1N2C]

### Step 1:

To a solution of 6c1 (Oakwood; 149 mg, 0.52 mmol) in dry DMF (1.0 mL) is added Cs₂CO₃ (170 mg, 0.52 mmol) and MeI (65 μL, 1.04 mmol). The mixture is stirred at RT for 16 h then is poured into water and brine before being extracted with EtOAc. The combined organic phases are dried (MgSO₄), filtered, concentrated to dryness and purified by flash chromatography (EtOAc:hexane) to give compound 6c2.

### Step 2:

A solution of 6c2 (101 mg, 0.34 mmol) in dry THF (1.0 mL) at 0° C. is treated with a 1.0 M LiHMDS solution in THF (0.51 mL, 0.51 mmol). The mixture is allowed to warm to RT and is treated with 4-fluoro-3-nitrobenzotrifluoride (Aldrich, 48 μL, 0.34 mmol) and allowed to stir for 3 h. The mixture is concentrated to dryness and the residue purified by flash chromatography (EtOAc:hexane) to afford compound 6c3.

### Step 3:

Compound 6c3 (81 mg, 0.17 mmol) is dissolved in AcOH (1.0 mL) and treated with iron powder (28 mg, 0.5 mmol) at 110° C. for 4 h. After cooling, the mixture is filtered and concentrated to dryness to give compound 6c4.

### Step 4:

Compound 6c4 (61 mg, 0.15 mmol) is treated with iodobenzene (93 μL, 0.83 mmol), K₂CO₃ (22 mg, 0.16 mmol) and CuI (9.3 mg, 0.05 mmol) in DMF (2 mL) at 130° C. for 16 h. The mixture is poured into water and extracted with EtOAc. The organic layer is washed with brine, dried (MgSO₄), filtered and concentrated to dryness. The residue is purified by flash chromatography (EtOAc/hexane), treated with TFA (1 mL) and after concentration, purified by preparative HPLC to give compound 6004.

### Example 6d

### Preparation of Compound 6005

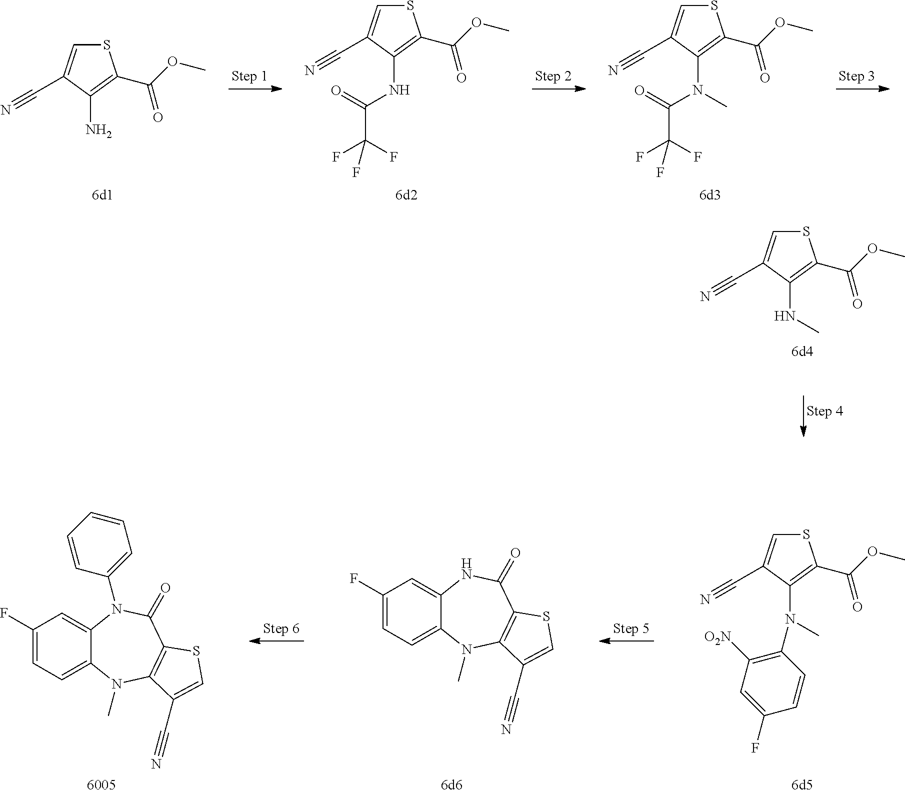

### Step 1:

Compound 6d1 (Maybridge; 2.0 g, 11 mmol) dissolved in DCM (40 mL) and pyridine (1.78 mL, 22 mmol) is treated dropwise with trifluoroacetic anhydride (2.33 mL, 16.5 mmol). After 1 h, the mixture is diluted with EtOAc and washed with aqueous 1 N HCl solution, aqueous 5% NaHCO₃ solution and brine. The organic phase is dried (MgSO₄), filtered and concentrated to give compound 6d2.

### Step 2:

To a solution of compound 6d2 (3.0 g, 10.8 mmol), Ph₃P (5.66 g, 21.6 mmol) in THF (100 mL) and MeOH (4.38 mL) is added DEAD (3.4 mL, 21.6 mmol) dropwise. The mixture is stirred at RT for 1 h before being concentrated under vacuum. The residue is purified flash chromatography (EtOAc:hexane) to give compound 6d3.

### Step 3:

A mixture of 6d3 (2.07 g, 7.1 mmol) and K₂CO₃ (2.9 g, 21.2 mmol) in anhydrous MeOH (35 mL) is heated to reflux for 30 min. The mixture is cooled and filtered and then concentrated. The residue diluted with EtOAc is washed with water and brine, dried (MgSO₄), filtered and concentrated to give compound 6d4.

### Step 4:

To a solution of compound 6d4 (1.26 g, 6.4 mmol) in dry DMF (20 mL) is added NaH (60% in oil, 385 mg, 9.6 mmol). After 15 min, 1,4-difluoro-2-nitrobenzene (Lancaster, 1.02 g, 6.4 mmol) is added. The mixture is stirred at RT for 1 h then aqueous 1 N HCl solution and EtOAc are added. The organic phase is washed with water and brine, dried (MgSO₄), filtered and concentrated. The residue is purified by flash chromatography (EtOAc/hexane) to give compound 6d5.

### Step 5:

Compound 6d5 (130 mg, 0.39 mmol) is treated with iron powder (217 mg, 0.39 mmol) in AcOH (4.0 mL) at 90° C. for 5 h. EtOAc is added and the mixture filtered. The filtrate is concentrated. The residue diluted with EtOAc is washed with water, aqueous 10% NaHCO₃ solution and brine. The organic phase is dried (MgSO₄), filtered and concentrated to give, after trituration with ether, compound 6d6.

### Step 6:

Using the procedure described in step 4 of example 6c, 6d6 is transformed into compound 6005.

### Example 7a

### Preparation of Compound 7001

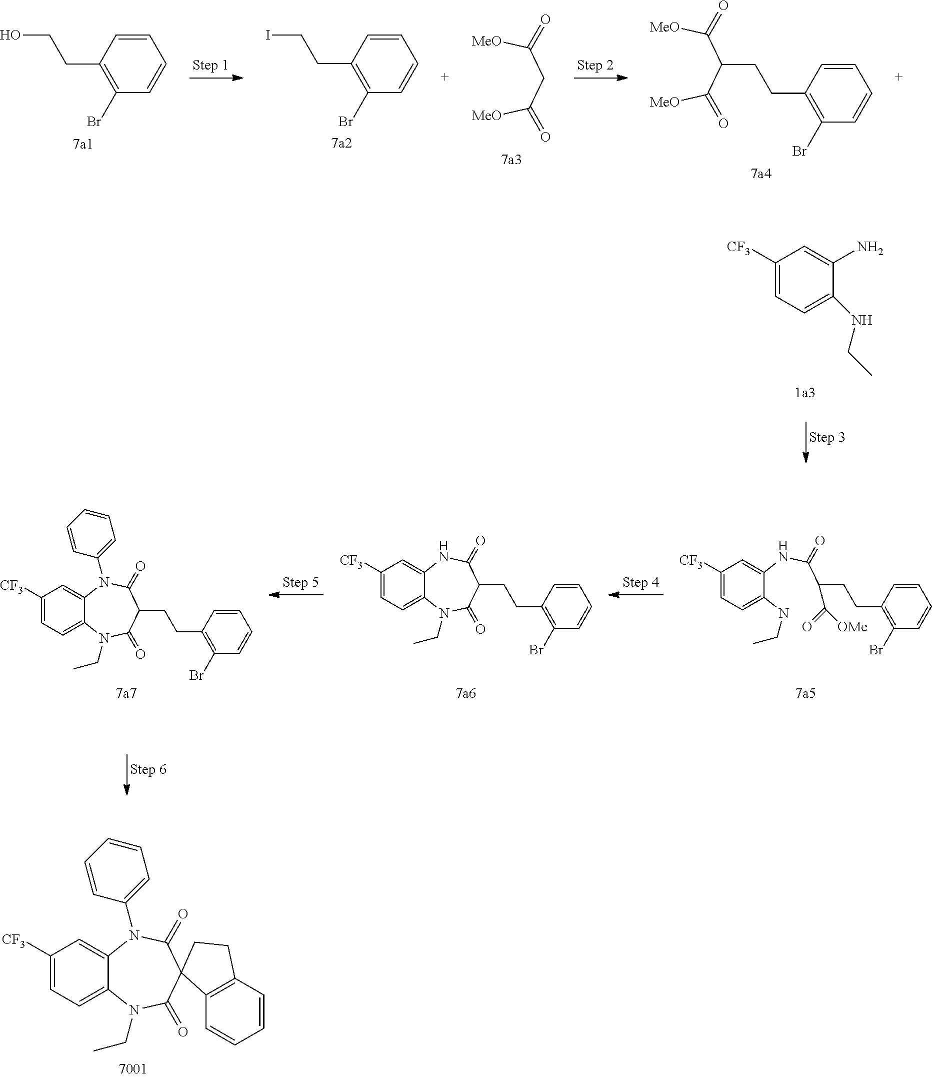

### Step 1:

PPh₃ (9.79 g, 36.9 mmol), imidazole (2.51 g, 36.9 mmol), and I₂ (9.37 g, 36.9 mmol) are successively added to a solution of alcohol 7a1 (Aldrich; 2.50 g, 12.3 mmol) in Et₂O (45 mL) and MeCN (15 mL). The reaction mixture is stirred at RT for 16 h. The mixture is filtered and the filtrate is diluted with Et₂O. The solution is successively washed with aqueous Na₂S₂O₃ solution, water and brine, dried (MgSO₄), filtered and concentrated under reduced pressure. The residue is purified by flash chromatography (DCM) to give compound 7a2.

### Step 2:

A solution of 7a2 (2.00 g, 6.43 mmol), 7a3 (6.17 mL, 54.0 mmol) and K₂CO₃ (6.33 g, 45.8 mmol) in DMF (50 mL) is heated to 80° C. for 24 h. The cooled mixture is filtered and the filtrate is diluted with EtOAc. The solution is successively washed with aqueous 1.0 N HCl solution, water and brine, dried (MgSO₄), filtered and concentrated under reduced pressure. The residue is purified by flash chromatography (hexane:EtOAC, 4:1) to give compound 7a4.

### Step 3:

A solution of 7a4 (5.90 g, 18.7 mmol) and 1a3 (3.82 g, 18.7 mmol) in DMF (11 mL) is heated to 170° C. for 1 h in a microwave. The reaction mixture is diluted with EtOAc and the solution is washed with water and brine, dried (MgSO₄), filtered and concentrated under reduced pressure. The residue is triturated with EtOAc to give compound 7a5.

### Step 4:

A solution of LiHMDS in THF (1.0 M; 34.5 mL, 34.5 mmol) is added to a solution of 7a5 (5.60 g, 11.5 mmol) in THF (100 mL) at RT. The mixture is stirred at RT for 45 min then is diluted with EtOAc. The solution is successively washed with aqueous 1.0 N HCl solution, water and brine, dried (MgSO₄), filtered and concentrated under reduced pressure. The residue is purified by flash chromatography (DCM:EtOAc, 19:1) to give compound 7a6.

### Step 5:

Ph₃Bi (2.23 g, 5.01 mmol), Cu(OAc)₂ (911 mg, 5.01 mmol) and pyridine (405 μL, 5.10 mmol) are added to a solution of 7a6 (1.27 g, 2.78 mmol) in DCM (60 mL) at RT. The mixture is stirred at RT for 4 h. The reaction mixture is diluted with DCM and the solution is washed with water and brine, dried (MgSO₄), filtered and concentrated under reduced pressure. The residue is purified by flash chromatography (DCM:EtOAc, 19:1) to give compound 7a7.

### Step 6:

A mixture of 7a7 (75.0 mg, 141 μmol), ((t-Bu)₃P)₂Pd (22 mg, 42 μmol) and Cs₂CO₃ (142 mg, 423 μmol) in THF (3.0 mL; degassed with Argon) is degassed with argon. (t-Bu)₃P (7.6 μL, 28 μmol) is added and the mixture (closed flask) is heated under argon to 80° C. for 24 h. The cooled mixture is filtered and diluted with EtOAc. The solution is washed with water and brine, dried (MgSO₄), filtered and concentrated under reduced pressure. The residue is dissolved with DMSO and purified by preparative HPLC to give compound 7001.

### Example 7b

### Preparation of Compounds 7002 and 7003

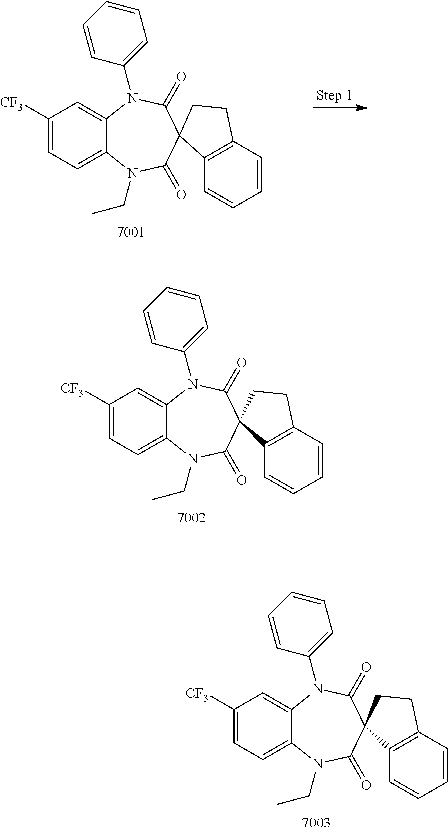 [SMILES: CCN1C(=O)C2(CCc3ccccc32)C(=O)N(c2ccccc2)c2cc(C(F)(F)F)ccc21 ; CCN1C(=O)[C@@]2(CCc3ccccc32)C(=O)N(c2ccccc2)c2cc(C(F)(F)F)ccc21 ; CCN1C(=O)[C@]2(CCc3ccccc32)C(=O)N(c2ccccc2)c2cc(C(F)(F)F)ccc21]

### Step 1:

A solution of racemic 7001 (15 mg, 33 μmol) in THF/MeCN (0.5/2.0 mL) is resolved by preparative HPLC using a CHIRALCEL OD column (20 μm, 5×50 cm; MeCN+0.06% TFA:water+0.06% TFA; 58:42; 60 mL/min, 100 min run time) to give compound 7002 (first eluting) and compound 7003.

### Example 7c

### Preparation of Compounds 7004 and 7018

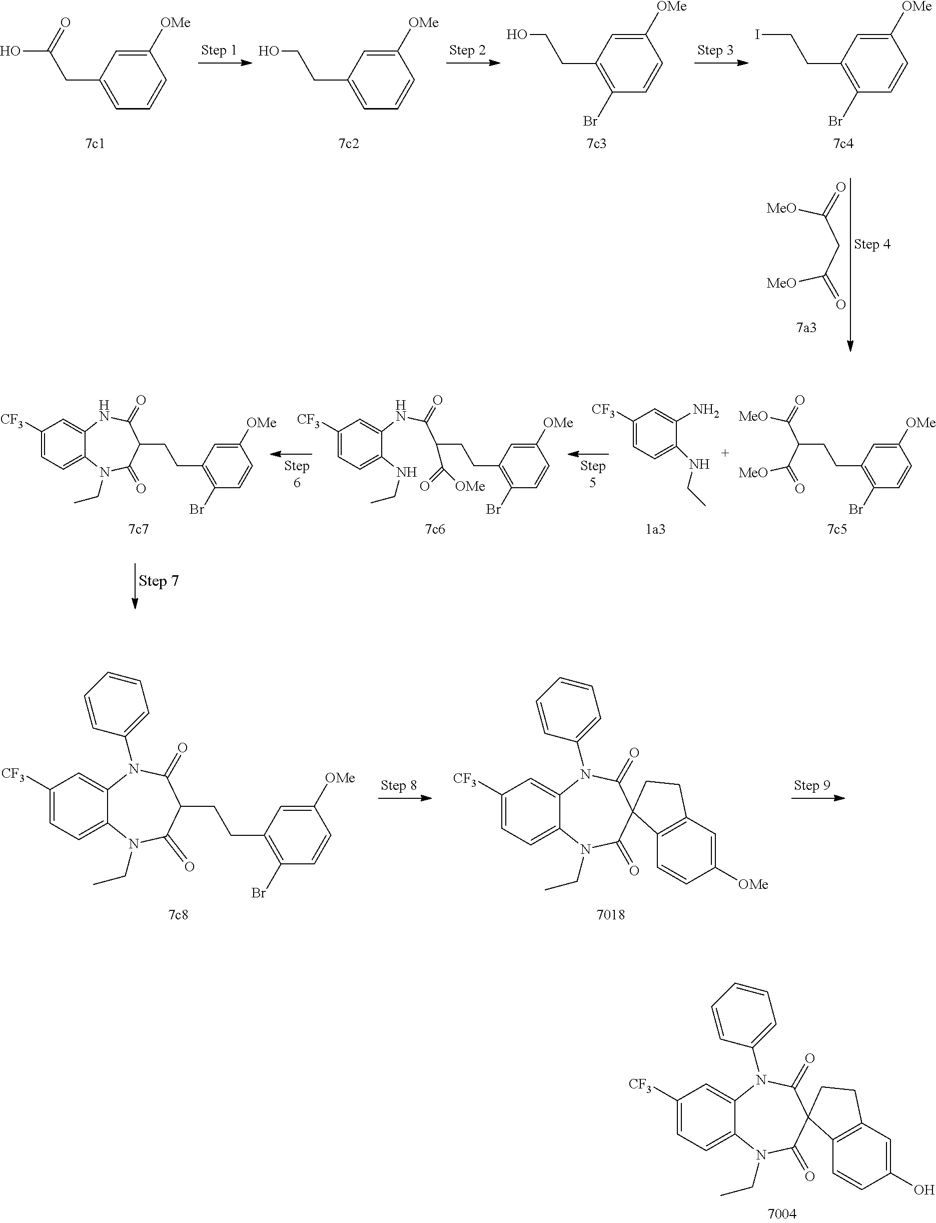

### Step 1:

A solution of 7c1 (Lancaster; 10.0 g, 60.2 mmol) in THF (10 mL) is added to an ice-cold solution of LiAlH₄ (4.71 g, 124 mmol) in THF (250 mL). The mixture is allowed to warm to RT and is stirred at RT for 24 h. The reaction mixture is carefully poured into an aqueous 0.1 N NaOH solution. The solution is extracted with Et₂O. The combined organic layers are washed with brine, dried (MgSO₄), filtered and concentrated under reduced pressure to give compound 7c2.

### Step 2:

Br₂ (6.85 mL, 134 mmol) is added dropwise to a solution of 7c2 (8.76 g, 57.5 mmol) in DCM (100 mL) and pyridine (5.59 mL, 69.0 mmol) at RT. The mixture is stirred for 16 h at RT then is diluted with DCM. The solution is washed with aqueous 20% Na₂S₂O₃ solution, water and brine, dried (MgSO₄), filtered and concentrated under reduced pressure. The residue is purified by flash chromatography (hexane:EtOAc, 4:1) to give compound 7c3.

### Step 3:

Using the procedure described in step 1 of example 7a, compound 7c3 is transformed into compound 7c4.

### Step 4:

Using the procedure described in step 2 of example 7a, compound 7c4 is transformed into compound 7c5.

### Step 5:

Using the procedure described in step 3 of example 7a, compound 7c5 is transformed into compound 7c6.

### Step 6:

Using the procedure described in step 4 of example 7a, compound 7c6 is transformed into compound 7c7.

### Step 7:

Using the procedure described in step 5 of example 7a, compound 7c7 is transformed into compound 7c8.

### Step 8:

Using the procedure described in step 6 of example 7a, compound 7c7 is transformed into compound 7018.

### Step 9:

A solution of 1.0 M BBr₃ in DCM (208 μL, 208 μmol) is added to an ice-cold solution of compound 7018 (50.0 mg, 104 mmol) in DCM (10 mL). The mixture is stirred at 0° C. for 45 min. An aqueous 1.0 N HCl solution is added and the mixture is diluted with EtOAc. The solution is washed with water and brine, dried (MgSO₄), filtered and concentrated under reduced pressure. The residue is purified by preparative HPLC to give compound 7004.

### Example 7d

### Preparation of Compound 7006

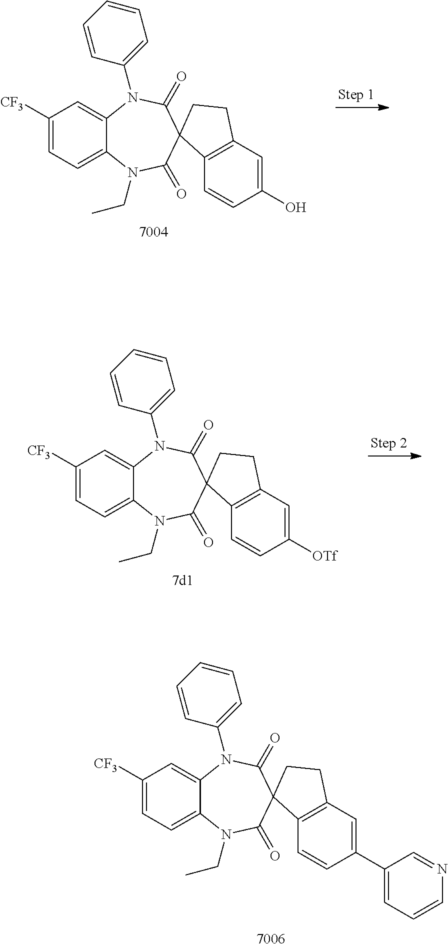 [SMILES: CCN1C(=O)C2(CCc3cc(O)ccc32)C(=O)N(c2ccccc2)c2cc(C(F)(F)F)ccc21 ; CCN1C(=O)C2(CCc3cc(OS(=O)(=O)C(F)(F)F)ccc32)C(=O)N(c2ccccc2)c2cc(C(F)(F)F)ccc21 ; CCN1C(=O)C2(CCc3cc(-c4cccnc4)ccc32)C(=O)N(c2ccccc2)c2cc(C(F)(F)F)ccc21]

### Step 1:

Tf₂O (352 μL, 2.09 mmol) is added to an ice-cold solution of compound 7004 (650 mg, 1.39 mmol) and Et₃N (583 μL, 4.18 mmol) in DCM (20 mL). The mixture is allowed to slowly warm to RT and is stirred at RT for 16 h. Aqueous saturated NaHCO₃ solution is added and the mixture is diluted with DCM. The solution is washed with water and brine, dried (MgSO₄), filtered and concentrated under reduced pressure. The residue is purified by flash chromatography (DCM:acetone, 19:1) to give compound 7d1.

### Step 2:

A mixture of 7d1 (40.0 mg, 66.8 mmol), 3-pyridineboronic acid (Aldrich, 20.5 mg, 167 mmol), aqueous 2.0 M Na₂CO₃ solution (167 μL, 334 μmol) and ((t-Bu)₃P)₂Pd (10 mg, 20 mmol) in DMF (2.0 mL) is heated to 145° C. for 17 min in a microwave. The cooled mixture is diluted with DMSO, filtered and purified by preparative HPLC to give compound 7006.

### Example 7e

### Preparation of Compounds 7033, 7034 and 7036

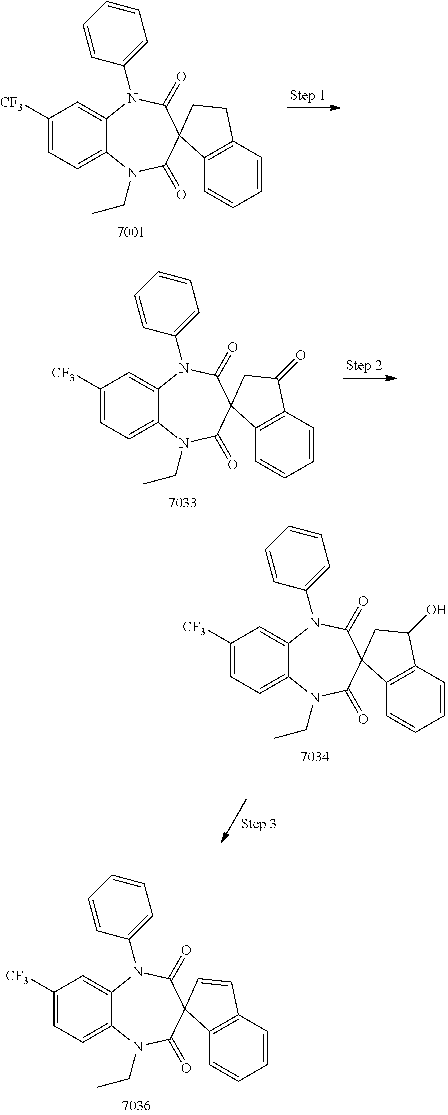 [SMILES: CCN1C(=O)C2(CCc3ccccc32)C(=O)N(c2ccccc2)c2cc(C(F)(F)F)ccc21 ; CCN1C(=O)C2(CC(=O)c3ccccc32)C(=O)N(c2ccccc2)c2cc(C(F)(F)F)ccc21 ; CCN1C(=O)C2(CC(O)c3ccccc32)C(=O)N(c2ccccc2)c2cc(C(F)(F)F)ccc21 ; CCN1C(=O)C2(C=Cc3ccccc32)C(=O)N(c2ccccc2)c2cc(C(F)(F)F)ccc21]

### Step 1:

A mixture of compound 7001 (367 mg, 815 mmol), KMnO₄ (932 mg, 5.90 mmol) and CuSO₄ (932 mg, 3.73 mmol) in DCM (50 mL) is heated to reflux for 3 days. The cooled mixture is filtered and concentrated under reduced pressure. The residue is purified by preparative HPLC to give compound 7033.

### Step 2:

NaBH₄ (8.1 mg, 0.21 mmol) is added to a solution of compound 7033 (50.0 mg, 0.108 mmol) in MeOH (1.5 mL) and DCM (1.5 mL) at RT. The mixture is stirred at RT for 2 h. Aqueous saturated NH₄Cl solution is added and the mixture is concentrated under reduced pressure. The residue is partitioned between EtOAc and water. The organic layer is washed with brine, dried (MgSO₄), filtered and concentrated under reduced pressure. The residue is purified by preparative HPLC to give compound 7034.

### Step 3:

A solution of compound 7034 (50.0 mg, 0.107 mmol) and p-TsOH.H₂O (25 mg) in benzene is heated to reflux for 1 h. The cooled reaction mixture is diluted with EtOAc. The resulting solution is washed with aqueous saturated NaHCO₃ solution, water and brine, dried (MgSO₄), filtered and concentrated under reduced pressure. The residue is purified by preparative HPLC to give compound 7036.

### Example 7f

### Preparation of Compound 7035

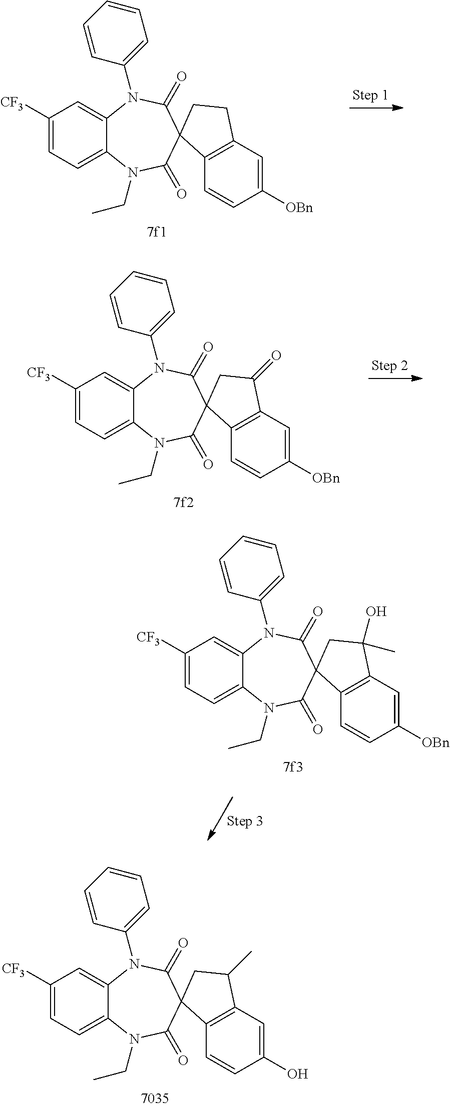 [SMILES: CCN1C(=O)C2(CCc3cc(OCc4ccccc4)ccc32)C(=O)N(c2ccccc2)c2cc(C(F)(F)F)ccc21 ; CCN1C(=O)C2(CC(=O)c3cc(OCc4ccccc4)ccc32)C(=O)N(c2ccccc2)c2cc(C(F)(F)F)ccc21 ; CCN1C(=O)C2(CC(C)(O)c3cc(OCc4ccccc4)ccc32)C(=O)N(c2ccccc2)c2cc(C(F)(F)F)ccc21 ; CCN1C(=O)C2(CC(C)c3cc(O)ccc32)C(=O)N(c2ccccc2)c2cc(C(F)(F)F)ccc21]

### Step 1:

CrO₃ (569 mg, 5.69 mmol) is added to a solution of 7f1 (prepared analogously to the procedure described for compound 7018 in example 7c, 1.62 g, 2.92 mmol) in AcOH (100 mL) at RT. After 20 min, the mixture is poured into water. The mixture is extracted with EtOAc. The combined organic layers are washed with water and brine, dried (MgSO₄), filtered and concentrated under reduced pressure. The residue is purified by flash chromatography (hexane:EtOAc, 7:3) to give compound 7f2.

### Step 2:

A 3.0 M solution of MeMgBr in Et₂O (105 μL, 315 mmol) is added dropwise to a cold (−78° C.) solution of 7f2 (75.0 mg, 131 μmol) in THF (4.0 mL). The reaction mixture is stirred at −78° C. for 1 h, then is allowed to warm to 0° C. and is stirred at this temperature for 1 h. Aqueous saturated NH₄Cl solution is added and the mixture is concentrated under reduced pressure. The residue is partitioned between EtOAc and water. The organic layer is washed with water and brine, dried (MgSO₄), filtered and concentrated under reduced pressure to give compound 7f3.

### Step 3:

A solution of 7f3 (50.0 mg, 85.2 mmol) and TFA (0.5 mL) in DCM (3.0 mL) is stirred at RT for 10 min. The mixture is concentrated under reduced pressure. The residue is dissolved in EtOH (10 mL) and 10% Pd/C (75 mg) is added and the mixture is stirred under a H₂ atmosphere (1 atm) for 2 h. The mixture is filtered and the filtrate is concentrated under reduced pressure. The residue is purified by preparative HPLC to give compound 7035.

### Example 7g

### Preparation of Compounds 7029 and 7032

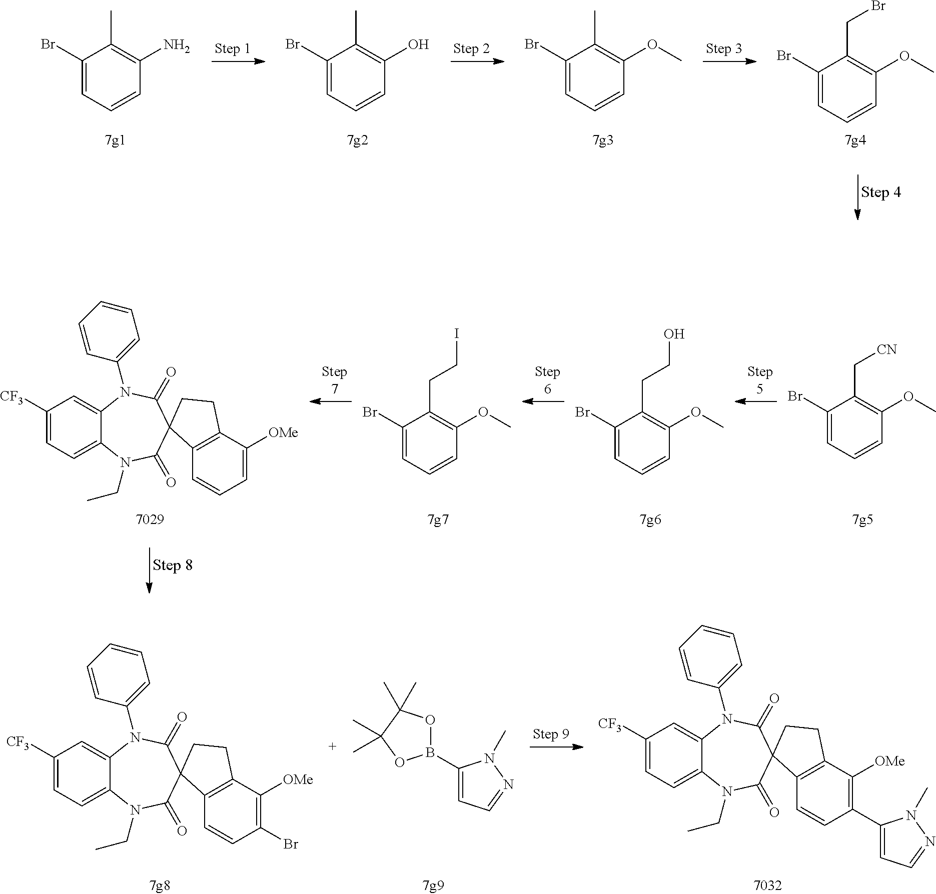

### Step 1:

A suspension of 7g1 (Alfa, 10 g, 54 mmol) in a mixture of concentrated sulfuric acid (130 mL), water (125 mL), and crushed ice (90 g) is stirred in an ice bath. Sodium nitrite solution in water (26 mL) is added dropwise while maintaining the temperature between 0 and 5° C. After the addition is complete, the mixture is stirred for 30 min at the same temperature. The mixture is poured into heated (120° C.) sulfuric acid (100 mL) and water (75 mL) and the heating is continued for 20 min. The cooled reaction mixture is extracted with Et₂O. The combined extracts are washed with water, brine, dried over MgSO₄, filtered and concentrated under vacuum to give compound 7g2.

### Step 2:

A mixture of 7g2 (9.2 g, 50 mmol) and K₂CO₃ (20 g, 150 mmol) in DMF (100 mL) is treated with iodomethane (9.2 mL, 150 mmol) and stirred at RT for 3 h. The mixture is diluted with EtOAc, washed with water followed by saturated aqueous NaCl. The organic layer is dried over MgSO₄ and the solvent is evaporated to give compound 7g3.

### Step 3:

A solution of 7g3, (9.3 g, 46 mmol), N-bromosuccinimide (8.2 g, 46 mmol) and benzoyl peroxide (450 mg, 1.8 mmol) in CCl₄ (100 mL) is heated to reflux and stirred for 6 h. The reaction mixture is cooled to RT, and then filtered. The filtrate is diluted with DCM and washed successively with 2.0 N NaOH, water and brine. The organic layer is dried over MgSO₄, filtered and concentrated under vacuum to give 7g4.

### Step 4:

A solution of 7g4 (11 g, 41 mmol) in DMSO (20 mL) is treated dropwise with a solution of sodium cyanide (3.5 g, 73 mmol) in DMSO (80 mL) and the reaction mixture is stirred for 3 h at RT. The reaction mixture is poured into water, extracted with Et₂O and the organic layer is successively washed with 6.0 N HCl, water and brine. The organic layer is dried over MgSO₄, filtered and concentrated under vacuum. The residue is purified by flash chromatography to give 7g5.

### Step 5:

A mixture of 7g5 (4.8 g, 21 mmol) in concentrated HCl/AcOH (1:1, 100 mL) is refluxed for 3 h and then cooled to RT. The resulting precipitate is collected by filtration and dried under high vacuum. The solid is dissolved in THF (50 mL) and added to a suspension of LiAlH₄ (1.4 g, 36 mmol) in THF (50 mL) at 0° C. The reaction mixture is allowed to come to RT and is then stirred for one day. The mixture is carefully poured into 0.1 N NaOH and extracted with Et₂O. The organic layer is washed with brine, dried over MgSO₄, filtered and concentrated under vacuum to give 7g6.

### Step 6:

Triphenylphosphine (3.0 g, 11 mmol), imidazole (760 mg, 11 mmol), and iodine (2.8 g, 11 mmol) are successively added to a solution of 7g6 (2.0 g, 8.6 mmol) dissolved in ether (45 mmol) and acetonitrile (15 mL). The reaction mixture is stirred at RT for 16 h. The reaction mixture is filtered and the filtrate is diluted with ether and washed successively with 20% aqueous sodium thiosulfate, water and brine. The organic layer is dried over MgSO₄, filtered and concentrated to dryness. The residue is purified by flash chromatography to give 7g7.

### Step 7:

Compound 7029 is prepared from 7g7 and 7a3 in analogy to the preparation of compound 7018 following steps 4 to 8 of example 7c.

### Step 8:

A solution of Br₂ (94 μL, 1.8 mmol) in AcOH (5 ml) is added to a solution of compound 7029 (400 mg, 0.832 mmol) in AcOH (25 mL). The mixture is stirred at RT for 16 h and concentrated under reduced pressure. The residue in solution in EtOAc is washed with an aqueous saturated Na₂S₂O₃ solution, water, an aqueous 1 N NaOH solution, water and brine. The residue is dried over MgSO₄, filtered and concentrated under reduced pressure. The residue is purified by flash chromatography to give compound 7g8.

### Step 9:

A mixture of 7g8 (25.0 mg, 44.7 mmol), 7g9 (Frontier, 23.2 mg, 112 μmol), aqueous 2.0 M Na₂CO₃ solution (112 μL, 224 μmol) and ((t-Bu)₃P)₂Pd (6.8 mg, 13 μmol) in DMF (2.0 mL) is heated to 145° C. for 17 min in a microwave. The cooled mixture is filtered and purified by preparative HPLC to give compound 7032.

### Example 7h

### Preparation of Compounds 7025 and 7026

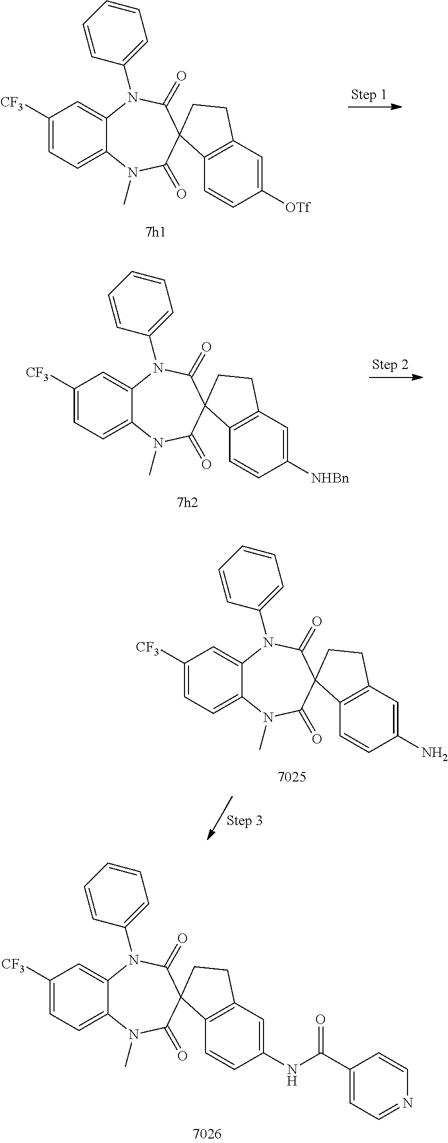

### Step 1:

Benzylamine (106 μL, 0.97 mmol) is added to a mixture of 7h1 (prepared analogously to the procedure described for compound 7d1 in examples 7c and 7d; 377 mg, 0.645 mmol), Cs₂CO₃ (212 mg, 0.651 mmol), Pd(OAc)₂ 23.0 mg, 0.102 mmol) and dicyclohexyl(2′,4′,6′-triisopropylbiphenyl-2-yl)phosphine (X-PHOS; 47.0 mg, 98.6 μmol) in DMF (3.0 mL) under a N₂ atmosphere. The mixture is heated to 150° C. for 15 min in a microwave. The cooled mixture is poured in water and extracted with EtOAc. The combined organic layers are washed with water and brine, dried (MgSO₄), filtered and concentrated under reduced pressure. The residue is purified by Combiflash (hexane:EtOAc) to give compound 7h2.

### Step 2:

A mixture of 7h2 (100 mg, 0.185 mmol) and 10% Pd(OH)₂/C (˜10 mg) in MeOH (15 mL) is stirred under a hydrogen atmosphere (1 atm) at RT for 18 h. The suspension is filtered through Celite® and the filtrate concentrated under reduced pressure. The residue is purified by preparative HPLC to give compound 7025.

### Step 3:

Et₃N (15.0 μL, 0.108 mmol) is added to a solution of compound 7025 (11.0 mg, 24.3 isonicotinic acid (Aldrich, 4.5 mg, 37 μmol) and HATU (15.0 mg, 39.4 μmol) in DCM (0.5 mL) at RT. The mixture is stirred at RT for 2 h, concentrated under reduced pressure and purified by preparative HPLC to give compound 7026.

### Example 8

### Capsid Assembly Assay; Capsid Disassembly Assay

The GAG polyprotein is the major structural protein required for HIV viral particle assembly. During the maturation process, Gag is cleaved by the viral protease and releases its four major proteins, matrix (MA), capsid (CA), nucleocapside (NC) and p6, as well as two spacer peptides termed SP1 and SP2. This triggers profound morphological changes to the viral particle as CA will reassemble to form the viral core, a structure that is essential for viral infectivity. The compounds of the invention inhibit HIV-1 capsid assembly as tested using an immobilized capsid assembly assay (CAA) and/or promote disassembly of assembled capsid complexes as tested using a capsid disassembly assay (CDA).

SEQ ID NO: 1 is a nucleic acid of 900 base pairs encoding HIV-1_{NL4-3} CA-NC, Gag residues 133-432, having the CA G94D mutation (SEQ ID NO: 2). SEQ ID NO: 1, is transferred to pET-11a expression vector (Novagen™) by PCR amplification using primers that introduce an NdeI site and a start codon at the 5′-end and a BamHI site and a stop codon at the 3′-end. Resistance mutations in CA were identified by passage of the HIV-1 virus in the presence of compounds of the invention, namely V27A/I, K30R, S33G, V36T, T58I, G61E, G208A/R, E213G, K30R/T58I, K30R/G208R, V36T/G208R, S33G/T58I, S33G/G208R and T58I/G208R, all numbered with reference to SEQ ID NO: 2,. These resistance mutations may be introduced in the expression vector using the QuikChange® II Site-Directed Mutagenesis Kit (Stratagene) according to the manufacturer's instructions. SEQ ID NOS: 2 is expressed in the BL21(DE3) E. coli cells (Novagen™). Briefly, LB media is inoculated with overnight pre-cultures and grown at 37° C. until mid log-phase (Abs600 ˜0.6), protein expression is induced by addition of 0.5-1 mM isopropyl-β,D-thiogalactopyranoside (IPTG) and carried out for 4 to 6 hours at 30° C. Cells are harvested by centrifugation and pellets are stored at −80° C. until purification.

Purification of CA-NC (SEQ ID NO: 2) is as follows: 5 to 10 g of cell paste are lysed by sonication in 40 ml of Buffer A [20 mM Tris pH 7.5; 1 μM ZnCl2; 10 mM β-mercaptoethanol] supplemented with 0.5M NaCl and Complete EDTA-free® protease inhibitors tablets (Roche). Nucleic acids and cell debris are removed by adding 0.11 volumes of 0.2 M ammonium sulfate and an equivalent volume of 10% poly-(ethyleneimine) pH 8.0, stirring the sample for 20 minutes at 4° C., followed by centrifugation at 30 000×g for 20 minutes. SEQ ID NO: 2 is recovered from the supernatant by adding 0.35 volumes of saturated ammonium sulfate solution followed by centrifugation at 10 000×g for 15 minutes. The pellet is dissolved in 10 ml of Buffer A+0.1 M NaCl and dialyzed overnight in the same buffer but at 0.05 M NaCl (Dialysis Buffer). The sample is then cleared by centrifugation and passed on a 1-ml HiTrapTMSP HP column (GE Healthcare) pre-equilibrated with Dialysis Buffer. SEQ ID NO: 2 is eluted with Buffer A+0.5 M NaCl, fractions containing the protein are pooled; concentration determination is done by Abs280 using the calculated molar extinction coefficient (E=40220 M-1 cm-1).

### Immobilized Capsid Assembly Assay (CAA)

Reacti-Bind Neutravidin Coated black 384-well plates (Pierce Cat#15402) are washed with 80 μl/well of Buffer A (50 mM Tris pH 8.0; 350 mM NaCl; 10 μM ZnSO4; 0.0025% CHAPS (w/v); 50 μg/ml bovine serum albumin (BSA); 1 mM DTT). Immobilization of a 5′-end biotin labeled (TG)25 oligonucleotide (Integrated DNA Technology Inc.) is carried out by adding 50 μl/well of a 25 nM solution of oligonucleotide in Buffer A+5 mg/ml of BSA (Sigma™) and incubating overnight. Unbound material is removed by two 80 μl/well washes with Buffer A. Assembly reactions are performed in 60 μl/well reactions comprising test compound, 100 nM of 5′-end fluorescein labeled (TG)25 oligonucleotide (Integrated DNA Technology Inc.) and 2 μM of CA-NC protein (SEQ ID NO: 2), using Buffer A+1% DMSO. Assembly reactions are incubated for 2 hours at room temperature and non-immobilized material is removed by two successive 80 μl/well washes with Buffer A. Finally, 80 μl/well of Buffer A+0.1% sodium dodecyl sulfate (SDS) is added and incubated for 15 minutes prior to quantification of captured fluorescence on a Victor2 plate reader (PerkinElmer Life Sciences) equipped with fluorescein excitation and emission filters using the standard manufacturer's settings for fluorescein fluorescence. The amount of captured fluorescence is considered to be proportional to the level of assembly. The IC50 values for each compound are generated by fitting inhibition curves from ten-point dilution series to the following equation:

% inhibition=((lmaxn×[l]n)÷([l]n+IC50n))×100, and represent the concentration of compound required for 50% inhibition of assembly.

### Capsid Disassembly Assay (CDA)

Reacti-Bind Neutravidin Coated black 384-well plates (Pierce Cat#15402) are washed with 80 μl/well of Buffer A (50 mM Tris pH 8.0; 350 mM NaCl; 10 μM ZnSO4; 0.0025% CHAPS (w/v); 50 μg/ml BSA; 1 mM DTT). Immobilization of a 5′-end biotin labeled (TG)25 oligonucleotide (Integrated DNA Technology Inc.) is carried out by adding 50 μl/well of a 25 nM solution of oligonucleotide in Buffer A+5 mg/ml BSA and incubating overnight. Unbound material is removed by two 80 μl/well washes with Buffer A. Assembly reactions are performed in 60 μl/well reactions comprising 100 nM of 5′-end fluorescein labeled (TG)25 oligonucleotide (Integrated DNA Technology Inc.) and 2 μM of CA-NC protein (SEQ ID NO: 2), using Buffer A. Assembly reactions are incubated for 2 hours at room temperature and non-immobilized material is removed by washing 80 μl/well with Buffer A. Test compounds, serially diluted in Buffer A+0.125% dimethyl sulfoxide (DMSO), are added to the wells (60 μl/well) and incubated at room temperature for 2 hours. Disassembled material is removed by two successive 80 μl/well washes with Buffer A. Finally, 80 μl/well of Buffer A+0.1% SDS is added and incubated for 15 minutes prior to quantification of captured fluorescence on a Victor2 plate reader (Perkin Elmer Life Sciences) equipped with fluorescein excitation and emission filters using manufacturer's setting for florescein fluorescence. The capacity of a test compound to dissociate assembled complexes is considered proportional to the observed loss of captured fluorescence. The IC50 values for each compound are generated by fitting disassembly curves from ten-point dilution series to the following equation: % inhibition=((lmaxn×[l]n)÷([l]n+IC50n))×100, and represent the concentration of compound required for 50% disassembly.

All compounds listed in Tables 1 to 7 are tested in the CAA or CDA described above, or both the CAA and CDA. The compounds show an IC₅₀ value in the range of 25 μM or less, and mostly in a range of 5 μM or less. Representative data is presented in the table below:

| | IC₅₀ (μM) | IC₅₀ (μM) |
|---|---|---|
| Cmpd # | CAA | CDA |
| 1004 | 0.47 | 0.38 |
| 1011 | 0.89 | 0.86 |
| 2002 | 0.73 | 0.48 |
| 2003 | 0.81 | 0.39 |
| 2009 | 0.91 | 0.14 |
| 2018 | 0.52 | 0.28 |
| 2030 | 0.39 | 0.32 |
| 2035 | 1.28 | |
| 2059 | 0.45 | 0.16 |
| 2076 | 1.60 | |
| 3011 | 0.63 | 0.18 |
| 3014 | 0.31 | 0.14 |
| 3027 | | 0.10 |
| 3034 | | 0.27 |
| 3055 | | 0.13 |
| 4002 | 0.71 | |
| 4007 | 0.31 | |
| 4032 | 0.55 | |
| 5001 | 0.36 | |
| 5003 | 0.26 | |
| 5005 | 0.32 | |
| 5026 | 0.17 | |
| 6001 | 1.00 | |
| 7001 | 0.73 | 0.46 |
| 7012 | | 0.20 |
| 7030 | | 0.17 |
| 7037 | | 0.30 |

### Example 9

### C8166 HIV-1 Luciferase Assay (EC₅₀)

The assay used to measure inhibition of HIV replication is as described in WO 2004/050643, pages 73-75, herein incorporated by reference, with the following modifications:

### Preparation of Compounds

Serial dilutions of HIV-1 inhibitors are prepared in complete media from DMSO stock solutions. Eleven serial dilutions of desired concentration are prepared in a 1 mL deep well titer plate (96 wells). The 12^{th} well contains complete media with no inhibitor and serves as the positive control. All samples contain the same concentration of DMSO 0.1% DMSO). Inhibitor is added, to triplicate wells, of a 96 well tissue culture treated clear view black microtiter plate (Corning Costar catalogue #3904). The total volume per well is 200 μL of media containing the cells and inhibitor. The last row is reserved for uninfected C8166 LTRluc cells to serve as the background blank control and the first row is media alone.

### Infection of Cells

Count C8166 LTRluc cells and place in a minimal volume of complete RPMI 1640 in a tissue culture flask (ex. 30×10⁶ cells in 10 mL media/25 cm² flask). Infect cells with HIV-1 at a moi of 0.005. Incubate cells for 1.5 hours at 37° C. on a rotating rack in a 5% CO₂ incubator. Resuspend cells in complete RPMI to give a final concentration of 25,000-cells/well. Add cells to wells of 96 well microtiter plate containing inhibitors. Add 25,000 uninfected C8166—LTRluc cells/well in 200 μL complete RPMI to last row for background control. Incubate cells at 37° C. in 5% CO₂ incubator for 3 days.

All compounds listed in Tables 1 to 7 are tested in the assay described in Example 8 or the assay described in Example 9 or both. Compounds tested in the assay of Example 9 showed EC₅₀ value in the range of 50 μM or less, and mostly in a range of 15 μM or less. Representative data in shown in the table below:

TABLES OF COMPOUNDS

| Cmpd # | EC₅₀ (μM) |
|---|---|
| 1004 | 12.7 |
| 1011 | 12.0 |
| 2002 | 2.6 |
| 2003 | 2.5 |
| 2009 | 0.9 |
| 2018 | 0.8 |
| 2030 | 0.4 |
| 2035 | 5.8 |
| 2059 | 0.07 |
| 2076 | 4.1 |
| 3011 | 0.6 |
| 3014 | 0.2 |
| 3027 | 0.7 |
| 3034 | 2.6 |
| 3055 | 0.4 |
| 4002 | 6.9 |
| 4007 | 7.8 |
| 4032 | 0.3 |
| 5001 | 1.5 |
| 5003 | 5.5 |
| 5005 | 0.4 |
| 5026 | 0.9 |
| 6001 | 8.1 |
| 7001 | 1.5 |
| 7012 | 0.2 |
| 7030 | 0.6 |
| 7037 | 1.0 |

The following tables list compounds representative of the invention. All of the compounds in Tables 1 to 7 are synthesized analogously to the Examples described above. For each compound in the tables, the analogous synthetic route to prepare each compound is identified by Example number. It will be apparent to a skilled person that the analogous synthetic routes may be used, with appropriate modifications, to prepare the compounds of the invention as described herein.

Retention times (t_{R}) for each compound are measured using the standard analytical UPLC conditions (Method A) described in the Examples unless otherwise indicated. The symbol, §, in the tables is used to indicate that the retention time (t_{R}) is measured by Method B. The symbol, 4, in the tables is used to indicate that the retention time (t_{R}) is measured by Method C. As is well known to one skilled in the art, retention time values are sensitive to the specific measurement conditions. Therefore, even if identical conditions of solvent, flow rate, linear gradient, and the like are used, the retention time values may vary when measured, for example, on different UPLC or HPLC instruments. Even when measured on the same instrument, the values may vary when measured, for example, using different individual UPLC or HPLC columns, or, when measured on the same instrument and the same individual column, the values may vary, for example, between individual measurements taken on different occasions.

TABLE 1

| | | | t_{R} | |
|---|---|---|---|---|
| Cmpd # | Structure | (M + H)⁺ | (min) | Example |
| 1001 | 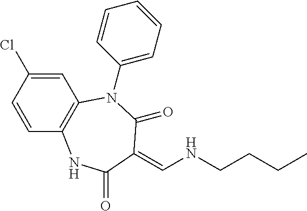 [SMILES: CCCCN/C=C1/C(=O)Nc2ccc(Cl)cc2N(c2ccccc2)C1=O] | 370.1, 372.0 | 2.06 | 1d |
| 1002 | 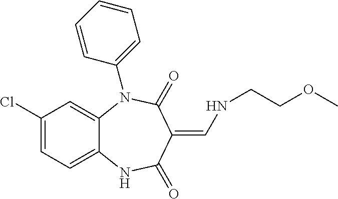 [SMILES: COCCN/C=C1/C(=O)Nc2ccc(Cl)cc2N(c2ccccc2)C1=O] | 372.0, 374.0 | 1.82 | 1b |
| 1003 | 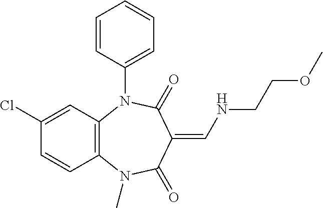 [SMILES: COCCN/C=C1/C(=O)N(C)c2ccc(Cl)cc2N(c2ccccc2)C1=O] | 386.1, 388.0 | 1.90 | 1b |
| 1004 | 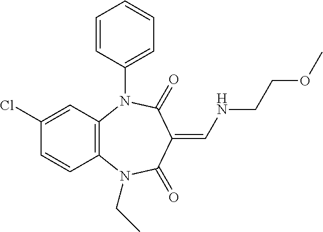 [SMILES: CCN1C(=O)/C(=C/NCCOC)C(=O)N(c2ccccc2)c2cc(Cl)ccc21] | 400.1, 402.0 | 1.97 | 1b |
| 1005 | 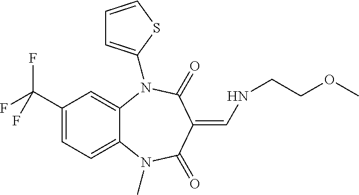 [SMILES: COCCN/C=C1/C(=O)N(C)c2ccc(C(F)(F)F)cc2N(c2cccs2)C1=O] | 426.4 | 1.94 | 1e |
| 1006 | 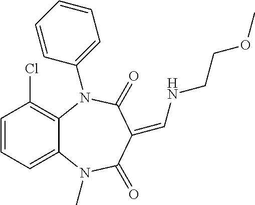 [SMILES: COCCN/C=C1/C(=O)N(C)c2cccc(Cl)c2N(c2ccccc2)C1=O] | 386.1, 388.0 | 1.82 | 1b |
| 1007 | 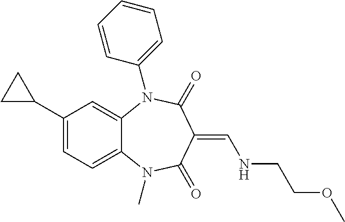 [SMILES: COCCN/C=C1\C(=O)N(C)c2ccc(C3CC3)cc2N(c2ccccc2)C1=O] | 392.2 | 1.94 | 1c |
| 1008 | 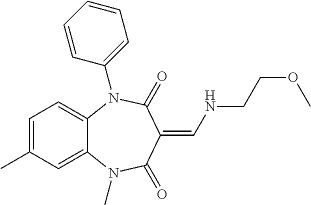 | 366.1 | 1.86 | 1c |
| 1009 | 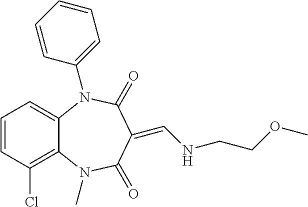 [SMILES: COCCN/C=C1\C(=O)N(C)c2c(Cl)cccc2N(c2ccccc2)C1=O] | 386.1, 388.0 | 1.85 | 1b |
| 1010 | 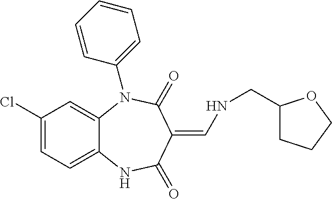 [SMILES: O=C1Nc2ccc(Cl)cc2N(c2ccccc2)C(=O)/C1=C\NCC1CCCO1] | 398.0, 400.0 | 1.88 | 1d |
| 1011 | 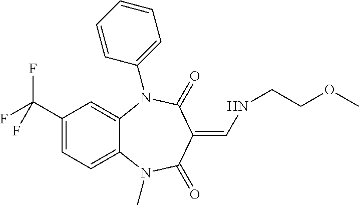 [SMILES: COCCN/C=C1/C(=O)N(C)c2ccc(C(F)(F)F)cc2N(c2ccccc2)C1=O] | 420.1 | 1.91 | 1c |

TABLE 2

| | | | t_{R} | |
|---|---|---|---|---|
| Cmpd # | Structure | (M + H)⁺ | (min) | Example |
| 2001 | 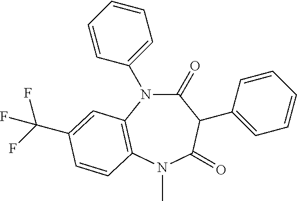 [SMILES: CN1C(=O)C(c2ccccc2)C(=O)N(c2ccccc2)c2cc(C(F)(F)F)ccc21] | 410.9 | 6.31 | 2a |
| 2002 | 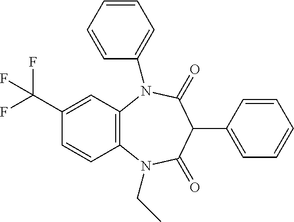 [SMILES: CCN1C(=O)C(c2ccccc2)C(=O)N(c2ccccc2)c2cc(C(F)(F)F)ccc21] | 424.9 | 8.10^{§} | 2a |
| 2003 | 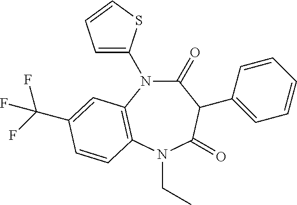 [SMILES: CCN1C(=O)C(c2ccccc2)C(=O)N(c2cccs2)c2cc(C(F)(F)F)ccc21] | 431.0 | 1.97 | 2b |
| 2004 | 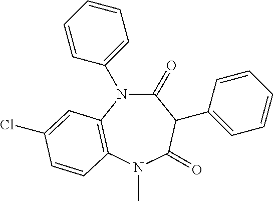 [SMILES: CN1C(=O)C(c2ccccc2)C(=O)N(c2ccccc2)c2cc(Cl)ccc21] | 377.0 | 1.90 | 2a |
| 2005 | 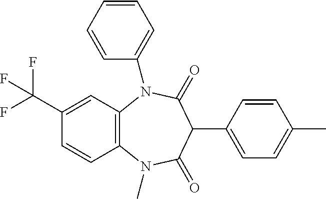 [SMILES: Cc1ccc(C2C(=O)N(C)c3ccc(C(F)(F)F)cc3N(c3ccccc3)C2=O)cc1] | 425.1 | 2.00 | 2a |
| 2006 | 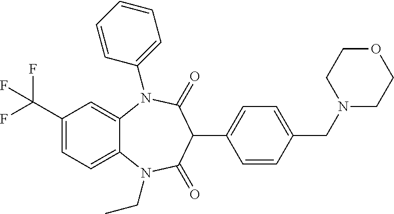 [SMILES: CCN1C(=O)C(c2ccc(CN3CCOCC3)cc2)C(=O)N(c2ccccc2)c2cc(C(F)(F)F)ccc21] | 524.2 | 1.66 | 2d |
| 2007 | 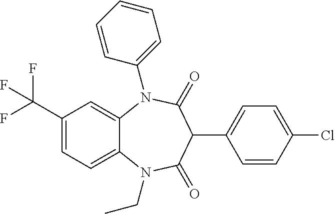 [SMILES: CCN1C(=O)C(c2ccc(Cl)cc2)C(=O)N(c2ccccc2)c2cc(C(F)(F)F)ccc21] | 459.1 | 2.06 | 2d |
| 2008 | 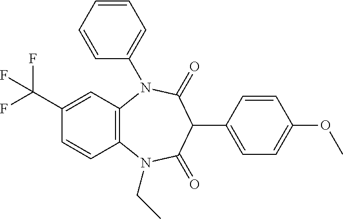 [SMILES: CCN1C(=O)C(c2ccc(OC)cc2)C(=O)N(c2ccccc2)c2cc(C(F)(F)F)ccc21] | 455.1 | 1.97 | 2c |
| 2009 | 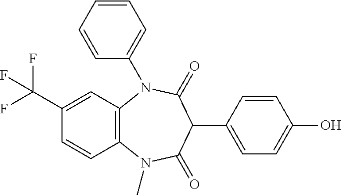 [SMILES: CN1C(=O)C(c2ccc(O)cc2)C(=O)N(c2ccccc2)c2cc(C(F)(F)F)ccc21] | 427.1 | 1.78 | 2a |
| 2010 | 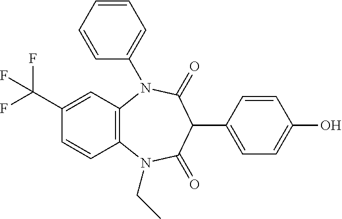 [SMILES: CCN1C(=O)C(c2ccc(O)cc2)C(=O)N(c2ccccc2)c2cc(C(F)(F)F)ccc21] | 441.1 | 1.84 | 2c |
| 2011 | 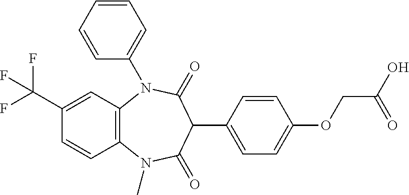 [SMILES: CN1C(=O)C(c2ccc(OCC(=O)O)cc2)C(=O)N(c2ccccc2)c2cc(C(F)(F)F)ccc21] | 485.1 | 1.69 | 2h |
| 2012 | 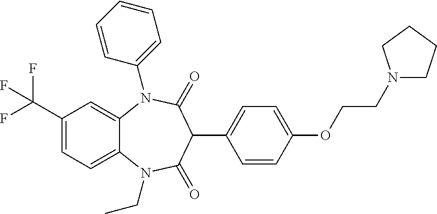 [SMILES: CCN1C(=O)C(c2ccc(OCCN3CCCC3)cc2)C(=O)N(c2ccccc2)c2cc(C(F)(F)F)ccc21] | 538.2 | 1.69 | 2d |
| 2013 | 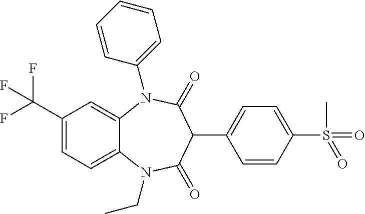 [SMILES: CCN1C(=O)C(c2ccc(S(C)(=O)=O)cc2)C(=O)N(c2ccccc2)c2cc(C(F)(F)F)ccc21] | 503.1 | 1.81 | 2d |
| 2014 | 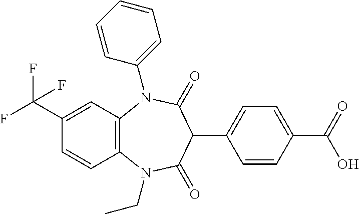 [SMILES: CCN1C(=O)C(c2ccc(C(=O)O)cc2)C(=O)N(c2ccccc2)c2cc(C(F)(F)F)ccc21] | 469.1 | 1.86 | 2e |
| 2015 | 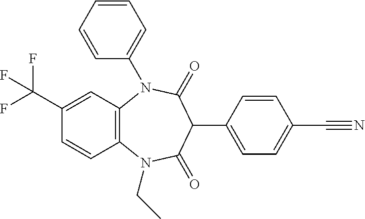 [SMILES: CCN1C(=O)C(c2ccc(C#N)cc2)C(=O)N(c2ccccc2)c2cc(C(F)(F)F)ccc21] | 450.1 | 1.91 | 2f |
| 2016 | 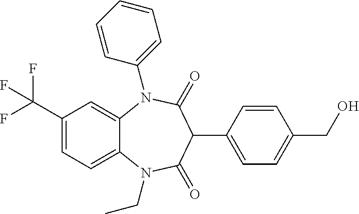 [SMILES: CCN1C(=O)C(c2ccc(CO)cc2)C(=O)N(c2ccccc2)c2cc(C(F)(F)F)ccc21] | 455.1 | 1.84 | 2d |
| 2017 | 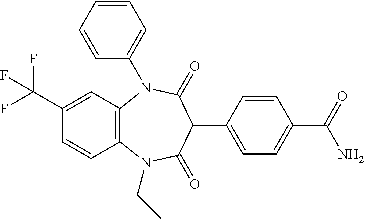 [SMILES: CCN1C(=O)C(c2ccc(C(N)=O)cc2)C(=O)N(c2ccccc2)c2cc(C(F)(F)F)ccc21] | 468.1 | 1.78 | 2f |
| 2018 | 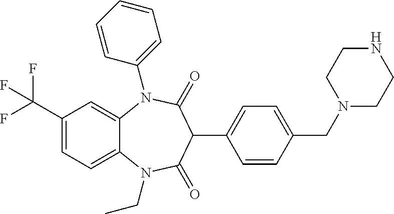 [SMILES: CCN1C(=O)C(c2ccc(CN3CCNCC3)cc2)C(=O)N(c2ccccc2)c2cc(C(F)(F)F)ccc21] | 523.2 | 1.69 | 2d |
| 2019 | 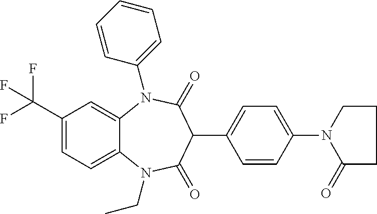 [SMILES: CCN1C(=O)C(c2ccc(N3CCCC3=O)cc2)C(=O)N(c2ccccc2)c2cc(C(F)(F)F)ccc21] | 508.1 | 1.88 | 2d |
| 2020 | 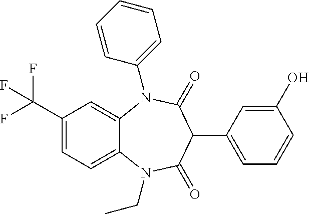 [SMILES: CCN1C(=O)C(c2cccc(O)c2)C(=O)N(c2ccccc2)c2cc(C(F)(F)F)ccc21] | 441.1 | 1.85 | 2d |
| 2021 | 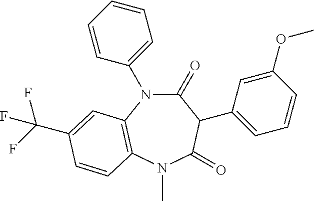 [SMILES: COc1cccc(C2C(=O)N(C)c3ccc(C(F)(F)F)cc3N(c3ccccc3)C2=O)c1] | 441.1 | 1.93 | 2a |
| 2022 | 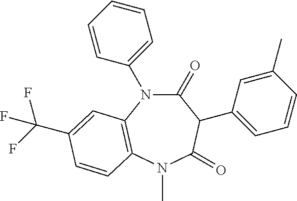 [SMILES: Cc1cccc(C2C(=O)N(C)c3ccc(C(F)(F)F)cc3N(c3ccccc3)C2=O)c1] | 425.1 | 2.00 | 2a |
| 2023 | 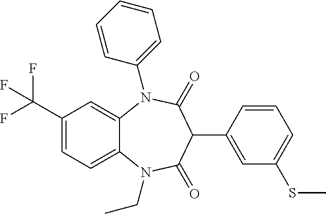 [SMILES: CCN1C(=O)C(c2cccc(SC)c2)C(=O)N(c2ccccc2)c2cc(C(F)(F)F)ccc21] | 471.1 | 2.06 | 2d |
| 2024 | 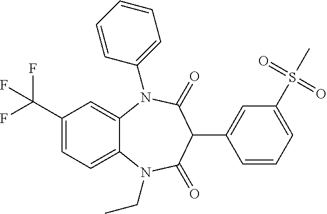 [SMILES: CCN1C(=O)C(c2cccc(S(C)(=O)=O)c2)C(=O)N(c2ccccc2)c2cc(C(F)(F)F)ccc21] | 501.0 (M − H)− | 1.81 | 2d |
| 2025 | 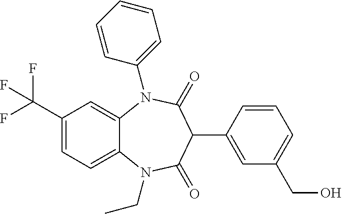 [SMILES: CCN1C(=O)C(c2cccc(CO)c2)C(=O)N(c2ccccc2)c2cc(C(F)(F)F)ccc21] | 455.1 | 1.85 | 2d |
| 2026 | 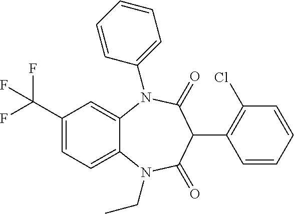 [SMILES: CCN1C(=O)C(c2ccccc2Cl)C(=O)N(c2ccccc2)c2cc(C(F)(F)F)ccc21] | 459.1 | 2.11 | 2d |
| 2027 | 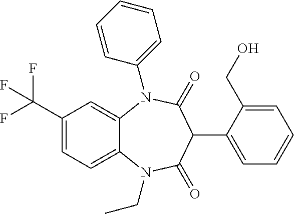 [SMILES: CCN1C(=O)C(c2ccccc2CO)C(=O)N(c2ccccc2)c2cc(C(F)(F)F)ccc21] | 455.2 | 6.30^{§} | 2d |
| 2028 | 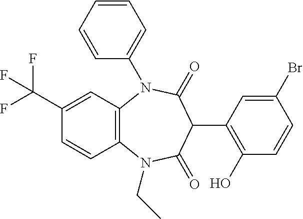 [SMILES: CCN1C(=O)C(c2cc(Br)ccc2O)C(=O)N(c2ccccc2)c2cc(C(F)(F)F)ccc21] | 519.0, 521.0 | 2.00 | 2c |
| 2029 | 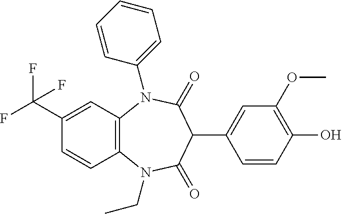 [SMILES: CCN1C(=O)C(c2ccc(O)c(OC)c2)C(=O)N(c2ccccc2)c2cc(C(F)(F)F)ccc21] | 471.1 | 1.86 | 2e |
| 2030 | 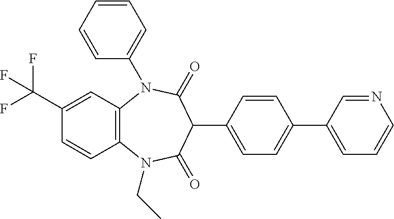 [SMILES: CCN1C(=O)C(c2ccc(-c3cccnc3)cc2)C(=O)N(c2ccccc2)c2cc(C(F)(F)F)ccc21] | 502.1 | 1.97 | 2f |
| 2031 | 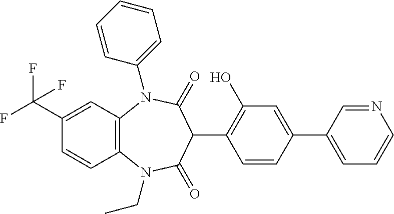 [SMILES: CCN1C(=O)C(c2ccc(-c3cccnc3)cc2O)C(=O)N(c2ccccc2)c2cc(C(F)(F)F)ccc21] | 518.1 | 1.93 | 2c |
| 2032 | 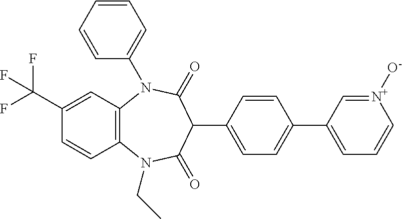 [SMILES: CCN1C(=O)C(c2ccc(-c3ccc[n+]([O-])c3)cc2)C(=O)N(c2ccccc2)c2cc(C(F)(F)F)ccc21] | 518.1 | 1.82 | 2f |
| 2033 | 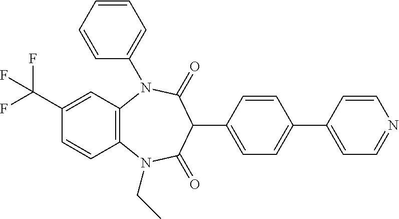 [SMILES: CCN1C(=O)C(c2ccc(-c3ccncc3)cc2)C(=O)N(c2ccccc2)c2cc(C(F)(F)F)ccc21] | 502.1 | 1.95 | 2f |
| 2034 | 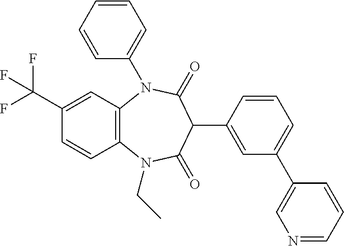 [SMILES: CCN1C(=O)C(c2cccc(-c3cccnc3)c2)C(=O)N(c2ccccc2)c2cc(C(F)(F)F)ccc21] | 502.1 | 1.98 | 2f |
| 2035 | 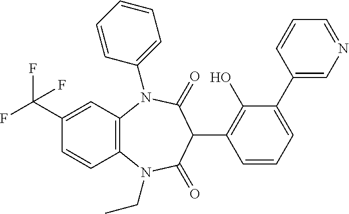 [SMILES: CCN1C(=O)C(c2cccc(-c3cccnc3)c2O)C(=O)N(c2ccccc2)c2cc(C(F)(F)F)ccc21] | 518.1 | 1.93 | 2c |
| 2036 | 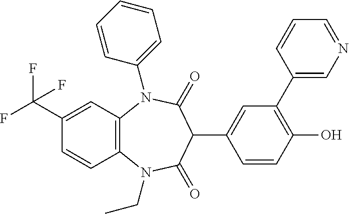 [SMILES: CCN1C(=O)C(c2ccc(O)c(-c3cccnc3)c2)C(=O)N(c2ccccc2)c2cc(C(F)(F)F)ccc21] | 518.0 | 4.74^{§} | 2f |
| 2037 | 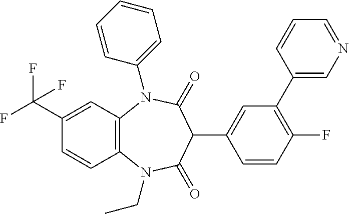 [SMILES: CCN1C(=O)C(c2ccc(F)c(-c3cccnc3)c2)C(=O)N(c2ccccc2)c2cc(C(F)(F)F)ccc21] | 520.1 | 2.00 | 2e |
| 2038 | 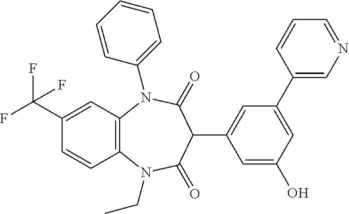 [SMILES: CCN1C(=O)C(c2cc(O)cc(-c3cccnc3)c2)C(=O)N(c2ccccc2)c2cc(C(F)(F)F)ccc21] | 518.1 | 5.20^{§} | 2e |
| 2039 | 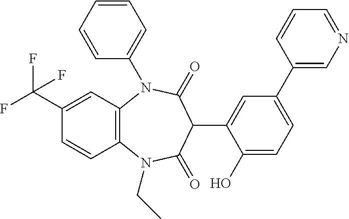 [SMILES: CCN1C(=O)C(c2cc(-c3cccnc3)ccc2O)C(=O)N(c2ccccc2)c2cc(C(F)(F)F)ccc21] | 518.1 | 1.91 | 2e |
| 2040 | 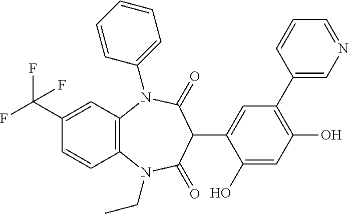 [SMILES: CCN1C(=O)C(c2cc(-c3cccnc3)c(O)cc2O)C(=O)N(c2ccccc2)c2cc(C(F)(F)F)ccc21] | 534.1 | 5.55^{§} | 2f |
| 2041 | 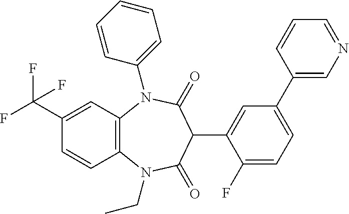 [SMILES: CCN1C(=O)C(c2cc(-c3cccnc3)ccc2F)C(=O)N(c2ccccc2)c2cc(C(F)(F)F)ccc21] | 520.1 | 2.05 | 2e |
| 2042 | 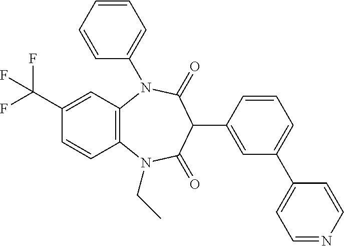 [SMILES: CCN1C(=O)C(c2cccc(-c3ccncc3)c2)C(=O)N(c2ccccc2)c2cc(C(F)(F)F)ccc21] | 502.1 | 1.96 | 2f |
| 2043 | 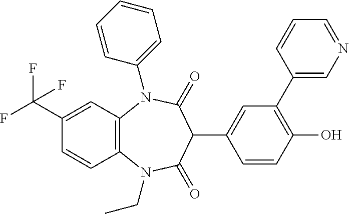 [SMILES: CCN1C(=O)C(c2ccc(O)c(-c3cccnc3)c2)C(=O)N(c2ccccc2)c2cc(C(F)(F)F)ccc21] | 518.1 | 1.83 | 2e |
| 2044 | 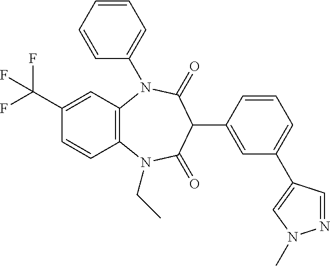 [SMILES: CCN1C(=O)C(c2cccc(-c3cnn(C)c3)c2)C(=O)N(c2ccccc2)c2cc(C(F)(F)F)ccc21] | 505.1 | 1.94 | 2f |
| 2045 | 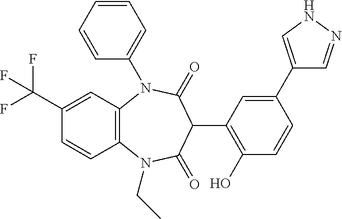 [SMILES: CCN1C(=O)C(c2cc(-c3cn[nH]c3)ccc2O)C(=O)N(c2ccccc2)c2cc(C(F)(F)F)ccc21] | 507.1 | 1.82 | 2e |
| 2046 | 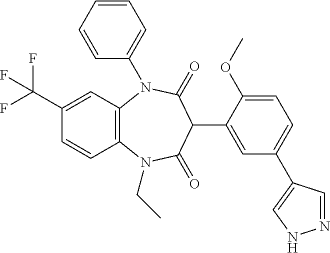 [SMILES: CCN1C(=O)C(c2cc(-c3cn[nH]c3)ccc2OC)C(=O)N(c2ccccc2)c2cc(C(F)(F)F)ccc21] | 521.1 | 1.97 | 2g |
| 2047 | 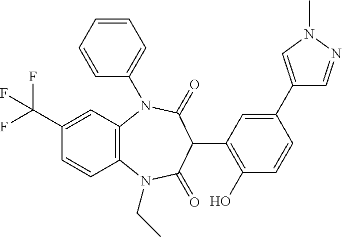 [SMILES: CCN1C(=O)C(c2cc(-c3cnn(C)c3)ccc2O)C(=O)N(c2ccccc2)c2cc(C(F)(F)F)ccc21] | 521.1 | 1.86 | 2e |
| 2048 | 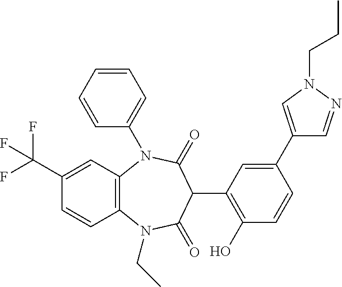 [SMILES: CCCn1cc(-c2ccc(O)c(C3C(=O)N(CC)c4ccc(C(F)(F)F)cc4N(c4ccccc4)C3=O)c2)cn1] | 549.2 | 1.97 | 2e |
| 2049 | 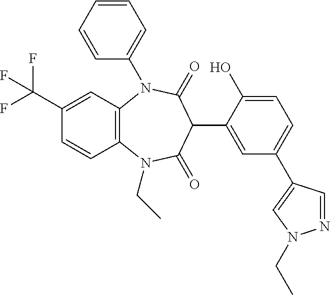 [SMILES: CCN1C(=O)C(c2cc(-c3cnn(CC)c3)ccc2O)C(=O)N(c2ccccc2)c2cc(C(F)(F)F)ccc21] | 535.1 | 1.91 | 2e |
| 2050 | 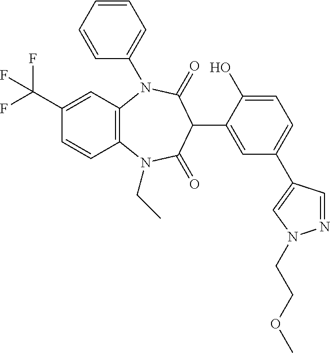 [SMILES: CCN1C(=O)C(c2cc(-c3cnn(CCOC)c3)ccc2O)C(=O)N(c2ccccc2)c2cc(C(F)(F)F)ccc21] | 565.2 | 1.88 | 2g |
| 2051 | 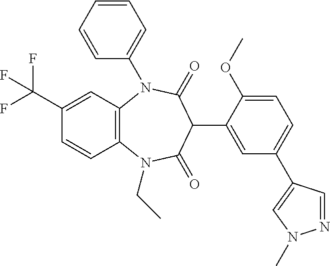 [SMILES: CCN1C(=O)C(c2cc(-c3cnn(C)c3)ccc2OC)C(=O)N(c2ccccc2)c2cc(C(F)(F)F)ccc21] | 535.2 | 2.00 | 2e |
| 2052 | 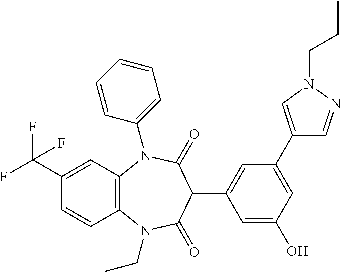 [SMILES: CCCn1cc(-c2cc(O)cc(C3C(=O)N(CC)c4ccc(C(F)(F)F)cc4N(c4ccccc4)C3=O)c2)cn1] | 549.2 | 1.94 | 2c |
| 2053 | 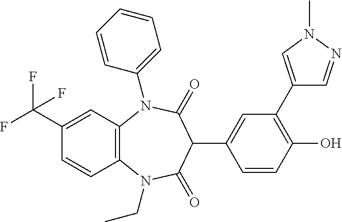 [SMILES: CCN1C(=O)C(c2ccc(O)c(-c3cnn(C)c3)c2)C(=O)N(c2ccccc2)c2cc(C(F)(F)F)ccc21] | 521.1 | 1.86 | 2e |
| 2054 | 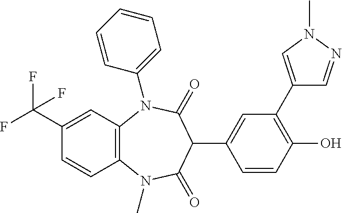 [SMILES: CN1C(=O)C(c2ccc(O)c(-c3cnn(C)c3)c2)C(=O)N(c2ccccc2)c2cc(C(F)(F)F)ccc21] | 507.1 | 1.80 | 2e |
| 2055 | 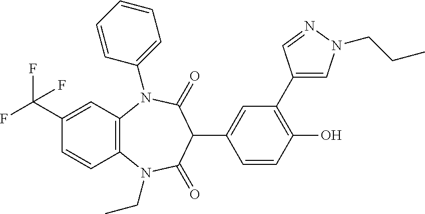 [SMILES: CCCn1cc(-c2cc(C3C(=O)N(CC)c4ccc(C(F)(F)F)cc4N(c4ccccc4)C3=O)ccc2O)cn1] | 549.2 | 6.58^{§} | 2e |
| 2056 | 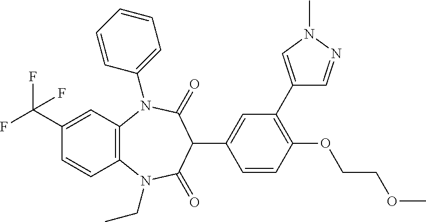 [SMILES: CCN1C(=O)C(c2ccc(OCCOC)c(-c3cnn(C)c3)c2)C(=O)N(c2ccccc2)c2cc(C(F)(F)F)ccc21] | 579.2 | 1.95 | 2e, 2c |
| 2057 | 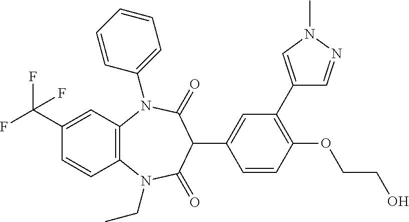 [SMILES: CCN1C(=O)C(c2ccc(OCCO)c(-c3cnn(C)c3)c2)C(=O)N(c2ccccc2)c2cc(C(F)(F)F)ccc21] | 565.2 | 1.88 | 2e |
| 2058 | 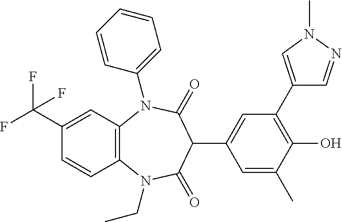 [SMILES: CCN1C(=O)C(c2cc(C)c(O)c(-c3cnn(C)c3)c2)C(=O)N(c2ccccc2)c2cc(C(F)(F)F)ccc21] | 535.2 | 6.58^{§} | 2e |
| 2059 | 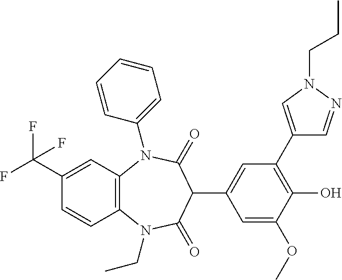 [SMILES: CCCn1cc(-c2cc(C3C(=O)N(CC)c4ccc(C(F)(F)F)cc4N(c4ccccc4)C3=O)cc(OC)c2O)cn1] | 579.2 | 1.99 | 2e |
| 2060 | 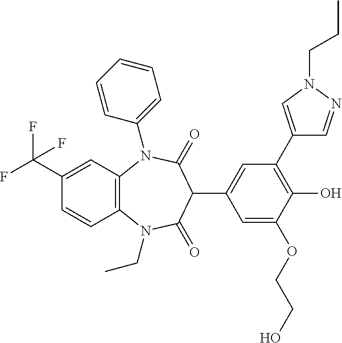 [SMILES: CCCn1cc(-c2cc(C3C(=O)N(CC)c4ccc(C(F)(F)F)cc4N(c4ccccc4)C3=O)cc(OCCO)c2O)cn1] | 609.2 | 1.95 | 2e |
| 2061 | 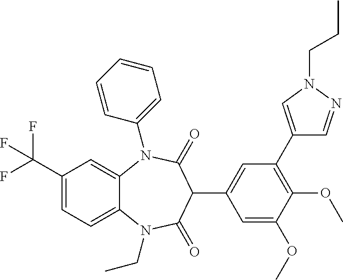 [SMILES: CCCn1cc(-c2cc(C3C(=O)N(CC)c4ccc(C(F)(F)F)cc4N(c4ccccc4)C3=O)cc(OC)c2OC)cn1] | 593.2 | 2.05 | 2e |
| 2062 | 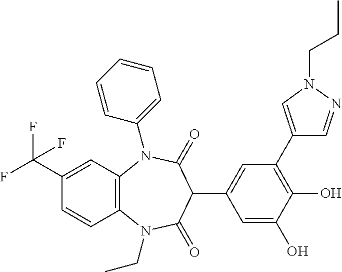 [SMILES: CCCn1cc(-c2cc(C3C(=O)N(CC)c4ccc(C(F)(F)F)cc4N(c4ccccc4)C3=O)cc(O)c2O)cn1] | 565.2 | 1.92 | 2e |
| 2063 | 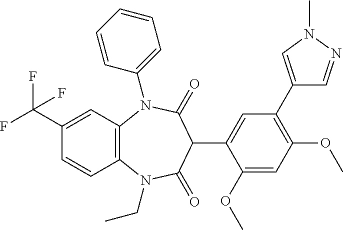 [SMILES: CCN1C(=O)C(c2cc(-c3cnn(C)c3)c(OC)cc2OC)C(=O)N(c2ccccc2)c2cc(C(F)(F)F)ccc21] | 565.2 | 1.99 | 2e |
| 2064 | 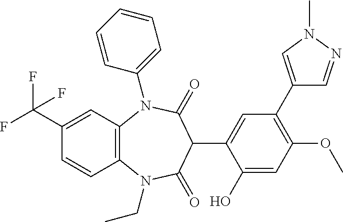 [SMILES: CCN1C(=O)C(c2cc(-c3cnn(C)c3)c(OC)cc2O)C(=O)N(c2ccccc2)c2cc(C(F)(F)F)ccc21] | 551.1 | 1.88 | 2e |
| 2065 | 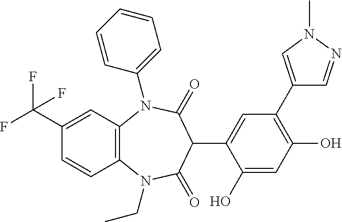 [SMILES: CCN1C(=O)C(c2cc(-c3cnn(C)c3)c(O)cc2O)C(=O)N(c2ccccc2)c2cc(C(F)(F)F)ccc21] | 537.1 | 1.82 | 2e |
| 2066 | 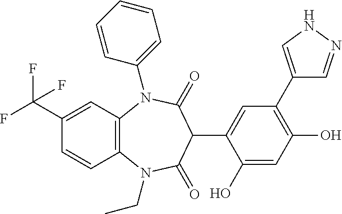 [SMILES: CCN1C(=O)C(c2cc(-c3cn[nH]c3)c(O)cc2O)C(=O)N(c2ccccc2)c2cc(C(F)(F)F)ccc21] | 523.1 | 1.77 | 2g |
| 2067 | 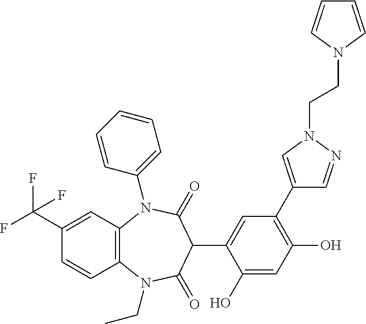 [SMILES: CCN1C(=O)C(c2cc(-c3cnn(CCn4cccc4)c3)c(O)cc2O)C(=O)N(c2ccccc2)c2cc(C(F)(F)F)ccc21] | 616.1 | 4.37^{§} | 2e, 2g |
| 2068 | 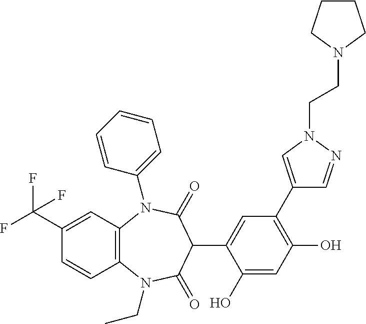 [SMILES: CCN1C(=O)C(c2cc(-c3cnn(CCN4CCCC4)c3)c(O)cc2O)C(=O)N(c2ccccc2)c2cc(C(F)(F)F)ccc21] | 620.2 | 1.68 | 2e, 2g |
| 2069 | 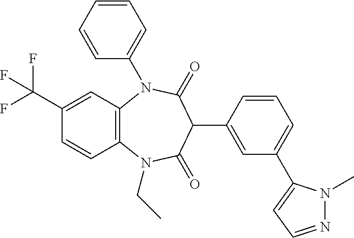 [SMILES: CCN1C(=O)C(c2cccc(-c3ccnn3C)c2)C(=O)N(c2ccccc2)c2cc(C(F)(F)F)ccc21] | 505.1 | 1.98 | 2f |
| 2070 | 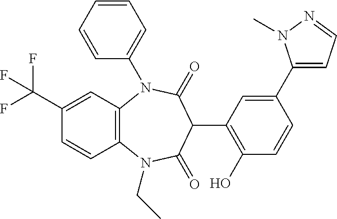 [SMILES: CCN1C(=O)C(c2cc(-c3ccnn3C)ccc2O)C(=O)N(c2ccccc2)c2cc(C(F)(F)F)ccc21] | 521.1 | 1.92 | 2e |
| 2071 | 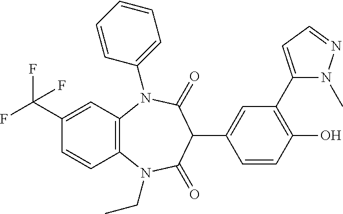 [SMILES: CCN1C(=O)C(c2ccc(O)c(-c3ccnn3C)c2)C(=O)N(c2ccccc2)c2cc(C(F)(F)F)ccc21] | 521.1 | 6.28^{§} | 2e |
| 2072 | 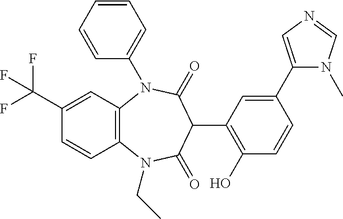 [SMILES: CCN1C(=O)C(c2cc(-c3cncn3C)ccc2O)C(=O)N(c2ccccc2)c2cc(C(F)(F)F)ccc21] | 521.1 | 1.68 | 2f |
| 2073 | 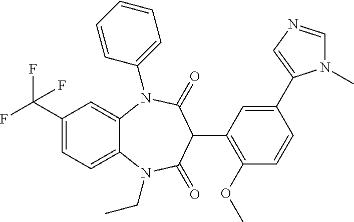 [SMILES: CCN1C(=O)C(c2cc(-c3cncn3C)ccc2OC)C(=O)N(c2ccccc2)c2cc(C(F)(F)F)ccc21] | 535.1 | 1.76 | 2f |
| 2074 | 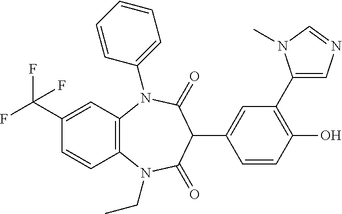 [SMILES: CCN1C(=O)C(c2ccc(O)c(-c3cncn3C)c2)C(=O)N(c2ccccc2)c2cc(C(F)(F)F)ccc21] | 521.2 | 3.78^{§} | 2e |
| 2075 | 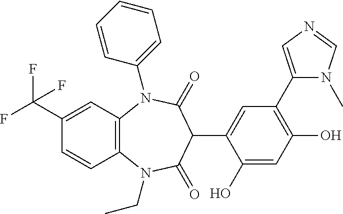 [SMILES: CCN1C(=O)C(c2cc(-c3cncn3C)c(O)cc2O)C(=O)N(c2ccccc2)c2cc(C(F)(F)F)ccc21] | 537.1 | 3.65^{§} | 2e |
| 2076 | 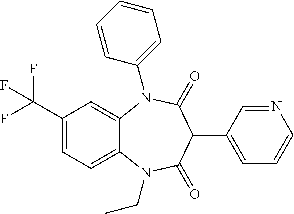 [SMILES: CCN1C(=O)C(c2cccnc2)C(=O)N(c2ccccc2)c2cc(C(F)(F)F)ccc21] | 426.1 | 1.79 | 2d |
| 2077 | 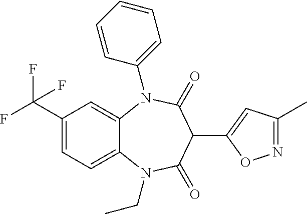 [SMILES: CCN1C(=O)C(c2cc(C)no2)C(=O)N(c2ccccc2)c2cc(C(F)(F)F)ccc21] | 430.1 | 1.84 | 2c |
| 2078 | 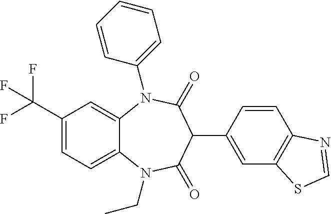 [SMILES: CCN1C(=O)C(c2ccc3ncsc3c2)C(=O)N(c2ccccc2)c2cc(C(F)(F)F)ccc21] | 482.1 | 1.92 | 2d |
| 2079 | 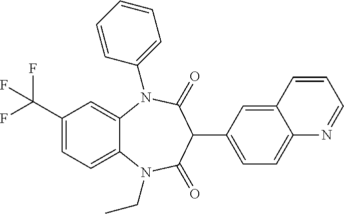 [SMILES: CCN1C(=O)C(c2ccc3ncccc3c2)C(=O)N(c2ccccc2)c2cc(C(F)(F)F)ccc21] | 476.1 | 1.91 | 2d |
| 2080 | 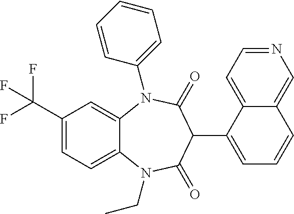 [SMILES: CCN1C(=O)C(c2cccc3cnccc23)C(=O)N(c2ccccc2)c2cc(C(F)(F)F)ccc21] | 476.1 | 1.93 | 2d |
| 2081 | 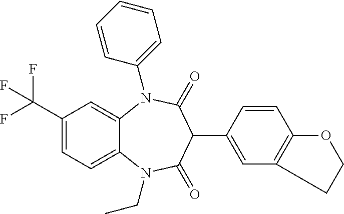 [SMILES: CCN1C(=O)C(c2ccc3c(c2)CCO3)C(=O)N(c2ccccc2)c2cc(C(F)(F)F)ccc21] | 467.1 | 1.98 | 2d |
| 2082 | 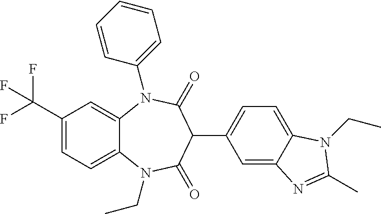 [SMILES: CCN1C(=O)C(c2ccc3c(c2)nc(C)n3CC)C(=O)N(c2ccccc2)c2cc(C(F)(F)F)ccc21] | 507.2 | 1.73 | 2d |

TABLE 3

| | | | t_{R} | |
|---|---|---|---|---|
| Cmpd # | Structure | (M + H)⁺ | (min) | Example |
| 3001 | 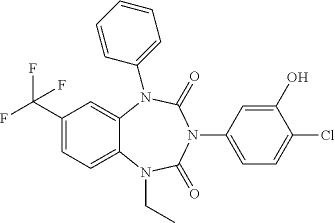 [SMILES: CCN1C(=O)N(c2ccc(Cl)c(O)c2)C(=O)N(c2ccccc2)c2cc(C(F)(F)F)ccc21] | 476.0, 478.0 | 6.90^{§} | 3a |
| 3002 | 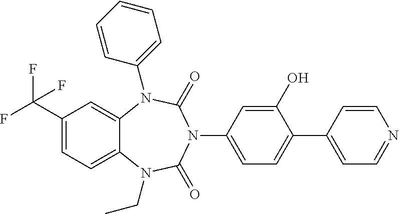 [SMILES: CCN1C(=O)N(c2ccc(-c3ccncc3)c(O)c2)C(=O)N(c2ccccc2)c2cc(C(F)(F)F)ccc21] | 519.2 | 1.99 | 3a |
| 3003 | 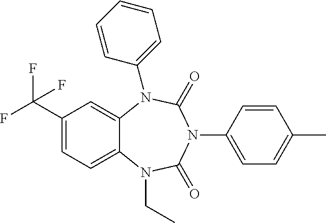 [SMILES: CCN1C(=O)N(c2ccc(C)cc2)C(=O)N(c2ccccc2)c2cc(C(F)(F)F)ccc21] | 440.2 | 2.15 | 3a |
| 3004 | 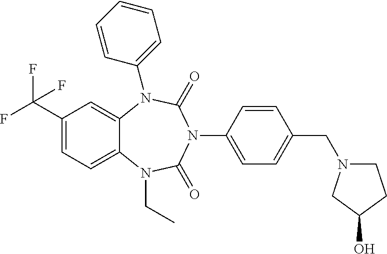 [SMILES: CCN1C(=O)N(c2ccc(CN3CC[C@@H](O)C3)cc2)C(=O)N(c2ccccc2)c2cc(C(F)(F)F)ccc21] | 525.3 | 1.73 | 3b |
| 3005 | 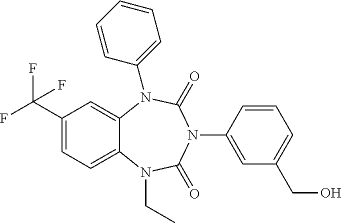 [SMILES: CCN1C(=O)N(c2cccc(CO)c2)C(=O)N(c2ccccc2)c2cc(C(F)(F)F)ccc21] | 456.2 | 1.96 | 3a |
| 3006 | 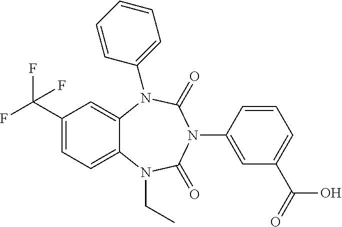 [SMILES: CCN1C(=O)N(c2cccc(C(=O)O)c2)C(=O)N(c2ccccc2)c2cc(C(F)(F)F)ccc21] | 470.2 | 1.97 | 3c |
| 3007 | 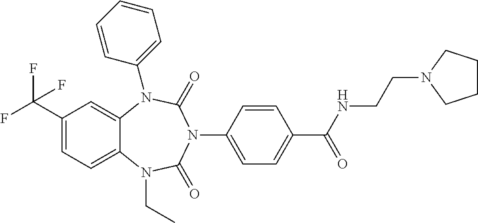 [SMILES: CCN1C(=O)N(c2ccc(C(=O)NCCN3CCCC3)cc2)C(=O)N(c2ccccc2)c2cc(C(F)(F)F)ccc21] | 566.3 | 1.78 | 3c |
| 3008 | 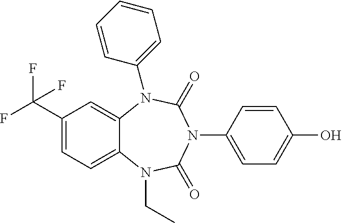 [SMILES: CCN1C(=O)N(c2ccc(O)cc2)C(=O)N(c2ccccc2)c2cc(C(F)(F)F)ccc21] | 525.3 | 1.8 | 3a |
| 3009 | 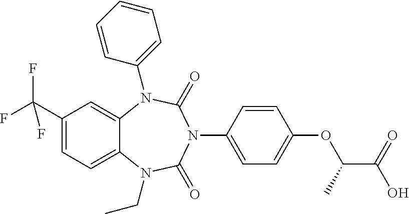 [SMILES: CCN1C(=O)N(c2ccc(O[C@@H](C)C(=O)O)cc2)C(=O)N(c2ccccc2)c2cc(C(F)(F)F)ccc21] | 514.2 | 1.92 | 3d |
| 3010 | 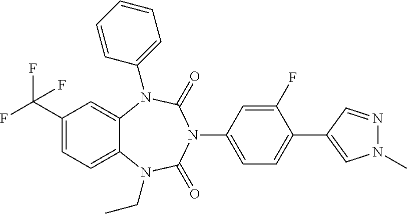 [SMILES: CCN1C(=O)N(c2ccc(-c3cnn(C)c3)c(F)c2)C(=O)N(c2ccccc2)c2cc(C(F)(F)F)ccc21] | 524.3 | 2.08 | 3e |
| 3011 | 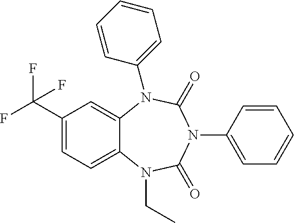 [SMILES: CCN1C(=O)N(c2ccccc2)C(=O)N(c2ccccc2)c2cc(C(F)(F)F)ccc21] | 426.1 | 2.09 | 3a |
| 3012 | 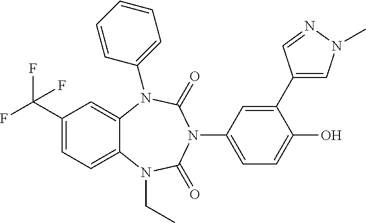 [SMILES: CCN1C(=O)N(c2ccc(O)c(-c3cnn(C)c3)c2)C(=O)N(c2ccccc2)c2cc(C(F)(F)F)ccc21] | 522.2 | 1.96 | 3a |
| 3013 | 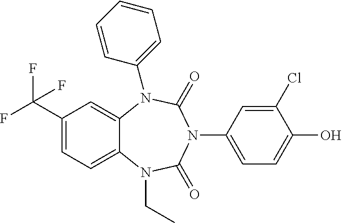 [SMILES: CCN1C(=O)N(c2ccc(O)c(Cl)c2)C(=O)N(c2ccccc2)c2cc(C(F)(F)F)ccc21] | 476.1, 478.0 | 2.05 | 3a |
| 3014 | 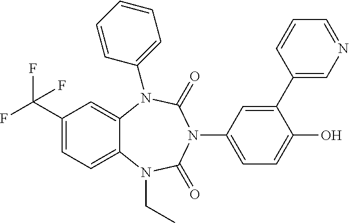 [SMILES: CCN1C(=O)N(c2ccc(O)c(-c3cccnc3)c2)C(=O)N(c2ccccc2)c2cc(C(F)(F)F)ccc21] | 519.1 | 5.56^{§} | 3a |
| 3015 | 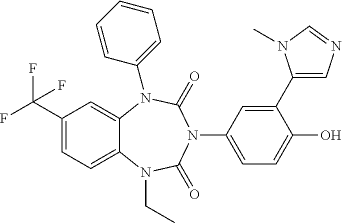 [SMILES: CCN1C(=O)N(c2ccc(O)c(-c3cncn3C)c2)C(=O)N(c2ccccc2)c2cc(C(F)(F)F)ccc21] | 522.2 | 1.78 | 3a |
| 3016 | 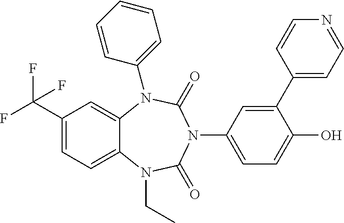 [SMILES: CCN1C(=O)N(c2ccc(O)c(-c3ccncc3)c2)C(=O)N(c2ccccc2)c2cc(C(F)(F)F)ccc21] | 519.2 | 1.97 | 3a |
| 3017 | 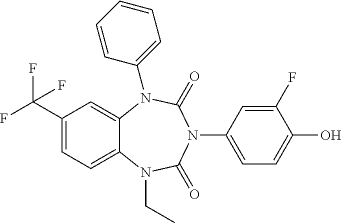 [SMILES: CCN1C(=O)N(c2ccc(O)c(F)c2)C(=O)N(c2ccccc2)c2cc(C(F)(F)F)ccc21] | 460.1 | 1.99 | 3a |
| 3018 | 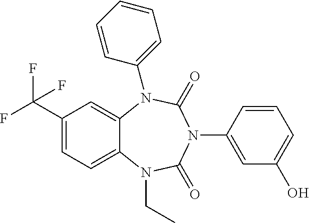 [SMILES: CCN1C(=O)N(c2cccc(O)c2)C(=O)N(c2ccccc2)c2cc(C(F)(F)F)ccc21] | 422.2 | 1.97 | 3a |
| 3019 | 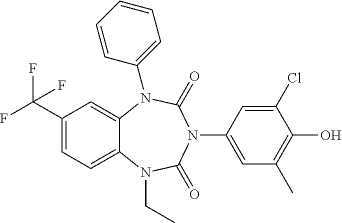 [SMILES: CCN1C(=O)N(c2cc(C)c(O)c(Cl)c2)C(=O)N(c2ccccc2)c2cc(C(F)(F)F)ccc21] | 490.0, 492.0 | 7.41^{§} | 3a |
| 3020 | 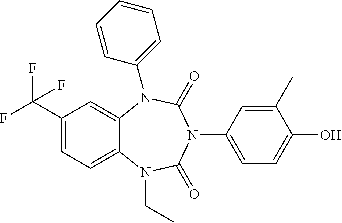 [SMILES: CCN1C(=O)N(c2ccc(O)c(C)c2)C(=O)N(c2ccccc2)c2cc(C(F)(F)F)ccc21] | 456.2 | 2.02 | 3a |
| 3021 | 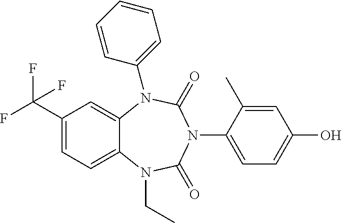 [SMILES: CCN1C(=O)N(c2ccc(O)cc2C)C(=O)N(c2ccccc2)c2cc(C(F)(F)F)ccc21] | 456.2 | 1.98 | 3a |
| 3022 | 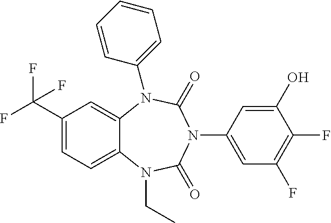 [SMILES: CCN1C(=O)N(c2cc(O)c(F)c(F)c2)C(=O)N(c2ccccc2)c2cc(C(F)(F)F)ccc21] | 478.1 | 2.06 | 3a |
| 3023 | 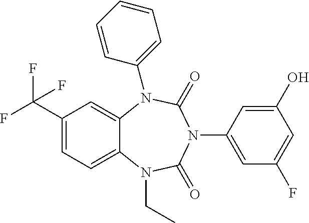 [SMILES: CCN1C(=O)N(c2cc(O)cc(F)c2)C(=O)N(c2ccccc2)c2cc(C(F)(F)F)ccc21] | 460.1 | 2.03 | 3a |
| 3024 | 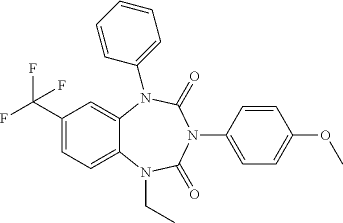 [SMILES: CCN1C(=O)N(c2ccc(OC)cc2)C(=O)N(c2ccccc2)c2cc(C(F)(F)F)ccc21] | 456.1 | 2.09 | 3a |
| 3025 | 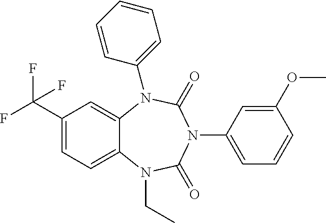 [SMILES: CCN1C(=O)N(c2cccc(OC)c2)C(=O)N(c2ccccc2)c2cc(C(F)(F)F)ccc21] | 456.2 | 2.10 | 3a |
| 3026 | 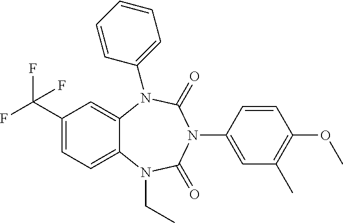 [SMILES: CCN1C(=O)N(c2ccc(OC)c(C)c2)C(=O)N(c2ccccc2)c2cc(C(F)(F)F)ccc21] | 470.2 | 2.16 | 3a |
| 3027 | 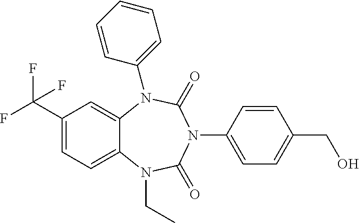 [SMILES: CCN1C(=O)N(c2ccc(CO)cc2)C(=O)N(c2ccccc2)c2cc(C(F)(F)F)ccc21] | 456.2 | 1.95 | 3a |
| 3028 | 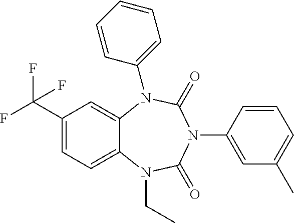 [SMILES: CCN1C(=O)N(c2cccc(C)c2)C(=O)N(c2ccccc2)c2cc(C(F)(F)F)ccc21] | 440.1 | 2.15 | 3a |
| 3029 | 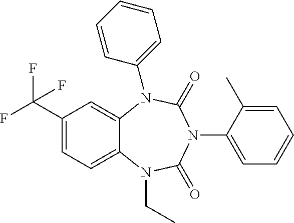 [SMILES: CCN1C(=O)N(c2ccccc2C)C(=O)N(c2ccccc2)c2cc(C(F)(F)F)ccc21] | 440.1 | 2.12 | 3a |
| 3030 | 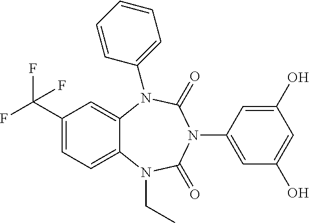 [SMILES: CCN1C(=O)N(c2cc(O)cc(O)c2)C(=O)N(c2ccccc2)c2cc(C(F)(F)F)ccc21] | 458.3 | 1.85 | 3a |
| 3031 | 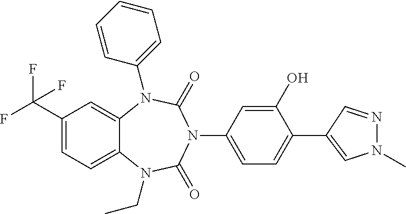 [SMILES: CCN1C(=O)N(c2ccc(-c3cnn(C)c3)c(O)c2)C(=O)N(c2ccccc2)c2cc(C(F)(F)F)ccc21] | 522.2 | 1.97 | 3a |
| 3032 | 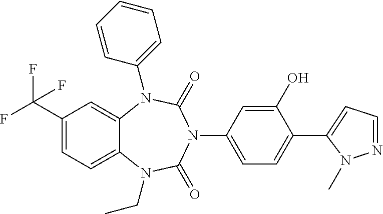 [SMILES: CCN1C(=O)N(c2ccc(-c3ccnn3C)c(O)c2)C(=O)N(c2ccccc2)c2cc(C(F)(F)F)ccc21] | 522.2 | 2.02 | 3a |
| 3033 | 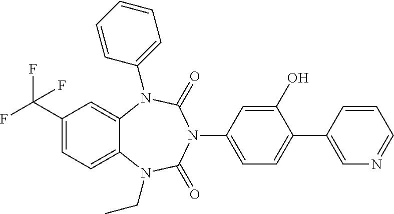 [SMILES: CCN1C(=O)N(c2ccc(-c3cccnc3)c(O)c2)C(=O)N(c2ccccc2)c2cc(C(F)(F)F)ccc21] | 519.2 | 2.01 | 3a |
| 3034 | 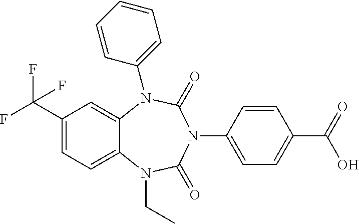 [SMILES: CCN1C(=O)N(c2ccc(C(=O)O)cc2)C(=O)N(c2ccccc2)c2cc(C(F)(F)F)ccc21] | 468.1 (M − H)− | 6.40^{§} | 3c |
| 3035 | 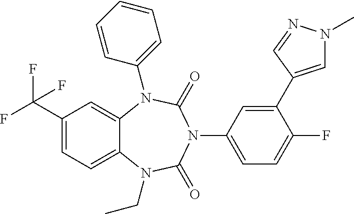 [SMILES: CCN1C(=O)N(c2ccc(F)c(-c3cnn(C)c3)c2)C(=O)N(c2ccccc2)c2cc(C(F)(F)F)ccc21] | 524.2 | 2.09 | 3e |
| 3036 | 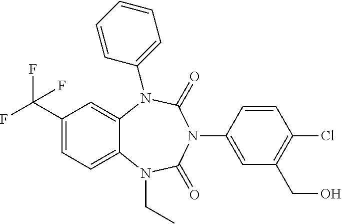 [SMILES: CCN1C(=O)N(c2ccc(Cl)c(CO)c2)C(=O)N(c2ccccc2)c2cc(C(F)(F)F)ccc21] | 490.2, 492.2 | 2.06 | 3a |
| 3037 | 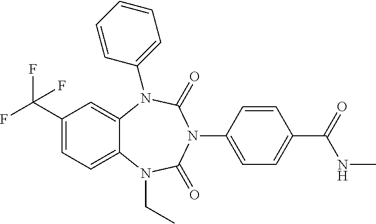 [SMILES: CCN1C(=O)N(c2ccc(C(=O)NC)cc2)C(=O)N(c2ccccc2)c2cc(C(F)(F)F)ccc21] | 483.2 | 1.93 | 3c |
| 3038 | 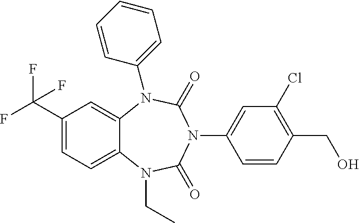 [SMILES: CCN1C(=O)N(c2ccc(CO)c(Cl)c2)C(=O)N(c2ccccc2)c2cc(C(F)(F)F)ccc21] | 490.2, 492.2 | 2.07 | 3a |
| 3039 | 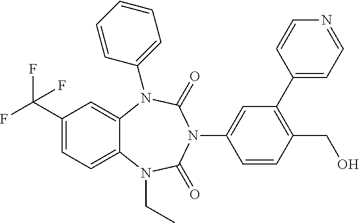 [SMILES: CCN1C(=O)N(c2ccc(CO)c(-c3ccncc3)c2)C(=O)N(c2ccccc2)c2cc(C(F)(F)F)ccc21] | 533.3 | 1.98 | 3a |
| 3040 | 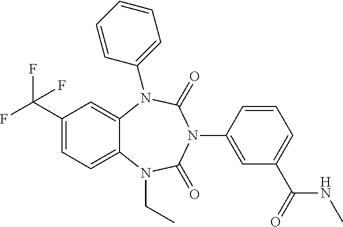 [SMILES: CCN1C(=O)N(c2cccc(C(=O)NC)c2)C(=O)N(c2ccccc2)c2cc(C(F)(F)F)ccc21] | 483.1 | 1.94 | 3c |
| 3041 | 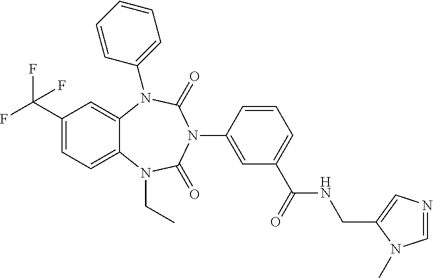 [SMILES: CCN1C(=O)N(c2cccc(C(=O)NCc3cncn3C)c2)C(=O)N(c2ccccc2)c2cc(C(F)(F)F)ccc21] | 563.2 | 1.79 | 3c |
| 3042 | 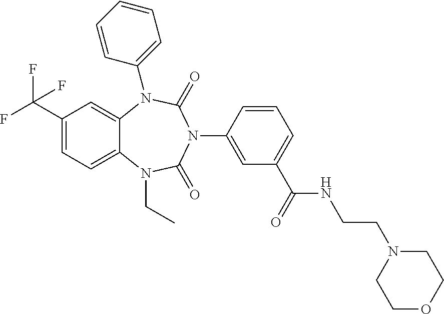 [SMILES: CCN1C(=O)N(c2cccc(C(=O)NCCN3CCOCC3)c2)C(=O)N(c2ccccc2)c2cc(C(F)(F)F)ccc21] | 582.3 | 1.80 | 3c |
| 3043 | 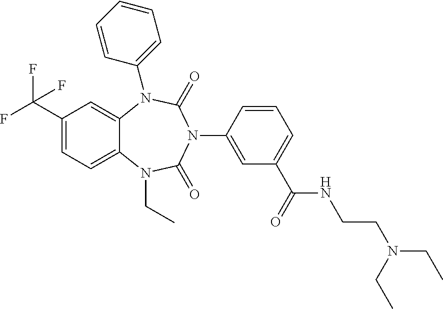 [SMILES: CCN(CC)CCNC(=O)c1cccc(N2C(=O)N(CC)c3ccc(C(F)(F)F)cc3N(c3ccccc3)C2=O)c1] | 568.3 | 1.79 | 3c |
| 3044 | 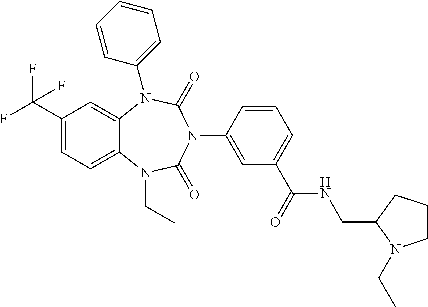 [SMILES: CCN1C(=O)N(c2cccc(C(=O)NCC3CCCN3CC)c2)C(=O)N(c2ccccc2)c2cc(C(F)(F)F)ccc21] | 580.3 | 1.80 | 3c |
| 3045 | 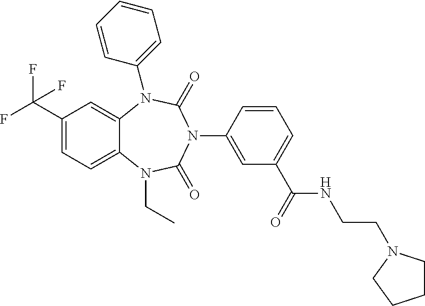 [SMILES: CCN1C(=O)N(c2cccc(C(=O)NCCN3CCCC3)c2)C(=O)N(c2ccccc2)c2cc(C(F)(F)F)ccc21] | 566.3 | 1.79 | 3c |
| 3046 | 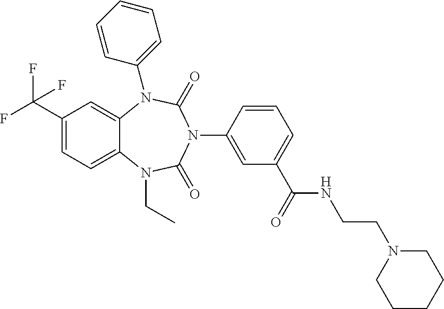 [SMILES: CCN1C(=O)N(c2cccc(C(=O)NCCN3CCCCC3)c2)C(=O)N(c2ccccc2)c2cc(C(F)(F)F)ccc21] | 580.3 | 1.81 | 3c |
| 3047 | 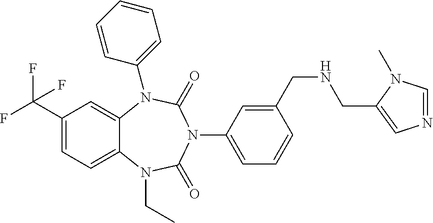 [SMILES: CCN1C(=O)N(c2cccc(CNCc3cncn3C)c2)C(=O)N(c2ccccc2)c2cc(C(F)(F)F)ccc21] | 549.3 | 1.72 | 3b |
| 3048 | 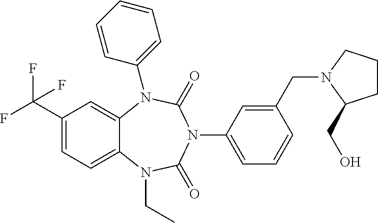 [SMILES: CCN1C(=O)N(c2cccc(CN3CCC[C@H]3CO)c2)C(=O)N(c2ccccc2)c2cc(C(F)(F)F)ccc21] | 539.3 | 1.77 | 3b |
| 3049 | 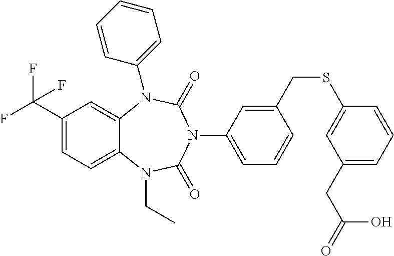 [SMILES: CCN1C(=O)N(c2cccc(CSc3cccc(CC(=O)O)c3)c2)C(=O)N(c2ccccc2)c2cc(C(F)(F)F)ccc21] | 606.2 | 2.16 | 3b |
| 3050 | 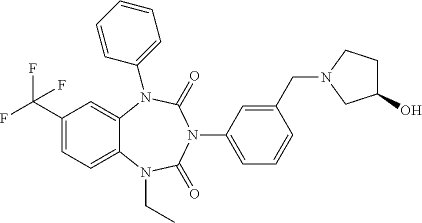 [SMILES: CCN1C(=O)N(c2cccc(CN3CC[C@@H](O)C3)c2)C(=O)N(c2ccccc2)c2cc(C(F)(F)F)ccc21] | 525.1 | 1.75 | 3b |
| 3051 | 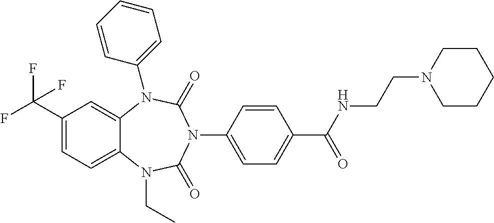 [SMILES: CCN1C(=O)N(c2ccc(C(=O)NCCN3CCCCC3)cc2)C(=O)N(c2ccccc2)c2cc(C(F)(F)F)ccc21] | 580.2 | 5.42^{§} | 3c |
| 3052 | 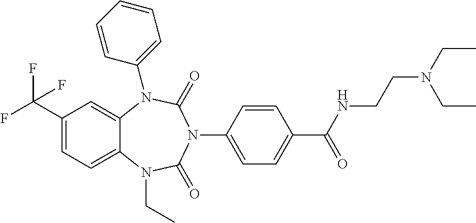 [SMILES: CCN(CC)CCNC(=O)c1ccc(N2C(=O)N(CC)c3ccc(C(F)(F)F)cc3N(c3ccccc3)C2=O)cc1] | 568.3 | 1.78 | 3c |
| 3053 | 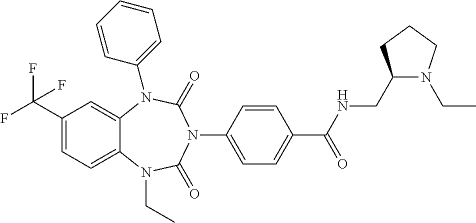 [SMILES: CCN1C(=O)N(c2ccc(C(=O)NC[C@H]3CCCN3CC)cc2)C(=O)N(c2ccccc2)c2cc(C(F)(F)F)ccc21] | 580.3 | 1.79 | 3c |
| 3054 | 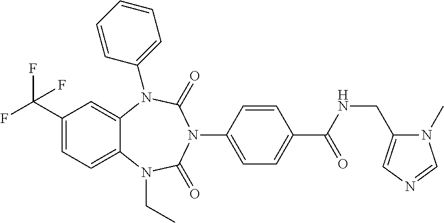 [SMILES: CCN1C(=O)N(c2ccc(C(=O)NCc3cncn3C)cc2)C(=O)N(c2ccccc2)c2cc(C(F)(F)F)ccc21] | 563.2 | 1.78 | 3c |
| 3055 | 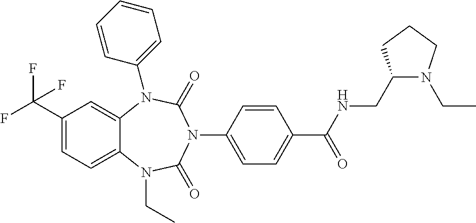 [SMILES: CCN1C(=O)N(c2ccc(C(=O)NC[C@@H]3CCCN3CC)cc2)C(=O)N(c2ccccc2)c2cc(C(F)(F)F)ccc21] | 580.3 | 1.80 | 3c |
| 3056 | 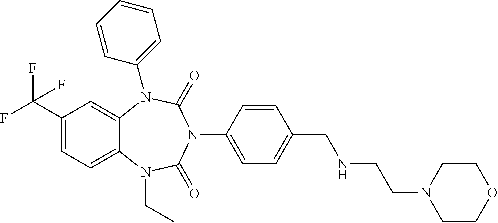 [SMILES: CCN1C(=O)N(c2ccc(CNCCN3CCOCC3)cc2)C(=O)N(c2ccccc2)c2cc(C(F)(F)F)ccc21] | 568.3 | 1.78 | 3b |
| 3057 | 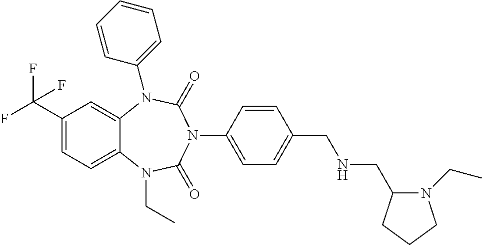 [SMILES: CCN1C(=O)N(c2ccc(CNCC3CCCN3CC)cc2)C(=O)N(c2ccccc2)c2cc(C(F)(F)F)ccc21] | 566.3 | 1.74 | 3b |
| 3058 | 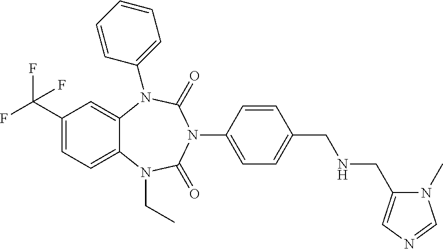 [SMILES: CCN1C(=O)N(c2ccc(CNCc3cncn3C)cc2)C(=O)N(c2ccccc2)c2cc(C(F)(F)F)ccc21] | 549.3 | 1.72 | 3b |
| 3059 | 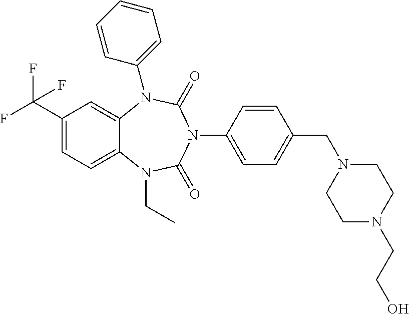 [SMILES: CCN1C(=O)N(c2ccc(CN3CCN(CCO)CC3)cc2)C(=O)N(c2ccccc2)c2cc(C(F)(F)F)ccc21] | 568.3 | 1.79 | 3b |
| 3060 | 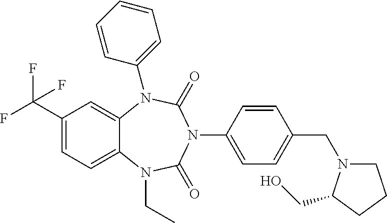 [SMILES: CCN1C(=O)N(c2ccc(CN3CCC[C@@H]3CO)cc2)C(=O)N(c2ccccc2)c2cc(C(F)(F)F)ccc21] | 539.3 | 1.74 | 3b |
| 3061 | 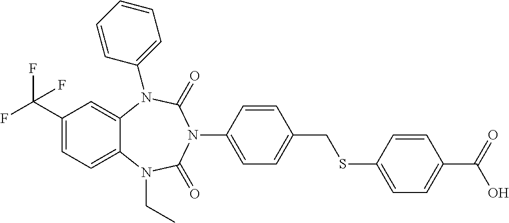 [SMILES: CCN1C(=O)N(c2ccc(CSc3ccc(C(=O)O)cc3)cc2)C(=O)N(c2ccccc2)c2cc(C(F)(F)F)ccc21] | 592.2 | 2.17 | 3b |
| 3062 | 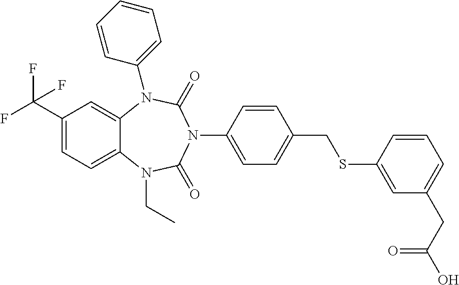 [SMILES: CCN1C(=O)N(c2ccc(CSc3cccc(CC(=O)O)c3)cc2)C(=O)N(c2ccccc2)c2cc(C(F)(F)F)ccc21] | 606.1 | 2.17 | 3b |
| 3063 | 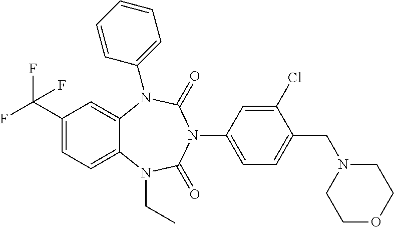 [SMILES: CCN1C(=O)N(c2ccc(CN3CCOCC3)c(Cl)c2)C(=O)N(c2ccccc2)c2cc(C(F)(F)F)ccc21] | 559.2, 561.2 | 2.08 | 3b |
| 3064 | 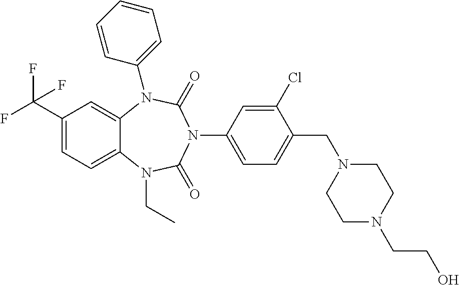 [SMILES: CCN1C(=O)N(c2ccc(CN3CCN(CCO)CC3)c(Cl)c2)C(=O)N(c2ccccc2)c2cc(C(F)(F)F)ccc21] | 602.2, 604.3 | 1.88 | 3b |
| 3065 | 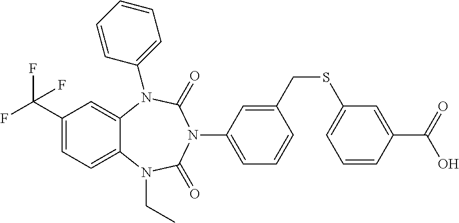 [SMILES: CCN1C(=O)N(c2cccc(CSc3cccc(C(=O)O)c3)c2)C(=O)N(c2ccccc2)c2cc(C(F)(F)F)ccc21] | 592.2 | 2.16 | 3b |
| 3066 | 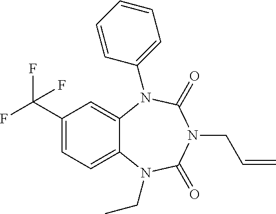 [SMILES: C=CCN1C(=O)N(CC)c2ccc(C(F)(F)F)cc2N(c2ccccc2)C1=O] | 390.3 | 6.8^{§} | 3f |
| 3067 | 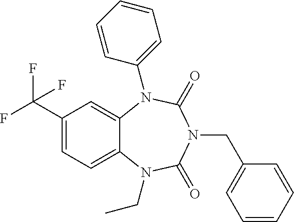 [SMILES: CCN1C(=O)N(Cc2ccccc2)C(=O)N(c2ccccc2)c2cc(C(F)(F)F)ccc21] | 440.2 | 7.3^{§} | 3f |
| 3068 | 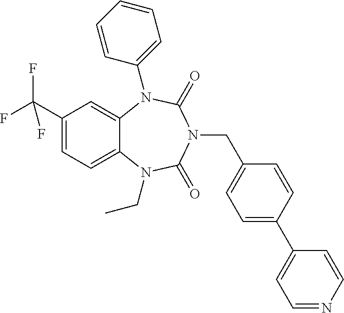 [SMILES: CCN1C(=O)N(Cc2ccc(-c3ccncc3)cc2)C(=O)N(c2ccccc2)c2cc(C(F)(F)F)ccc21] | 517.1 | 5.6^{§} | 3f |
| 3069 | 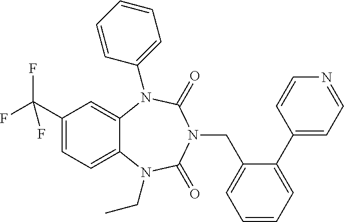 [SMILES: CCN1C(=O)N(Cc2ccccc2-c2ccncc2)C(=O)N(c2ccccc2)c2cc(C(F)(F)F)ccc21] | 517.1 | 5.4^{§} | 3f |
| 3070 | 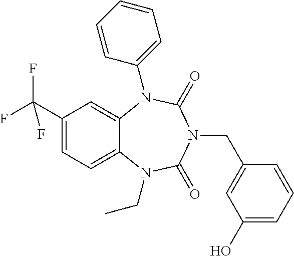 [SMILES: CCN1C(=O)N(Cc2cccc(O)c2)C(=O)N(c2ccccc2)c2cc(C(F)(F)F)ccc21] | 456.1 | 6.3^{§} | 3f |

TABLE 4

| | | | t_{R} | |
|---|---|---|---|---|
| Cmpd # | Structure | (M + H)⁺ | (min) | Example |
| 4001 | 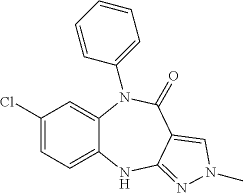 [SMILES: Cn1cc2c(n1)Nc1ccc(Cl)cc1N(c1ccccc1)C2=O] | 325.3, 327.2 | 1.83 | 4a |
| 4002 | 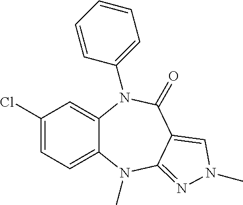 [SMILES: CN1c2ccc(Cl)cc2N(c2ccccc2)C(=O)c2cn(C)nc21] | 339.3, 341.3 | 1.93 | 4b |
| 4003 | 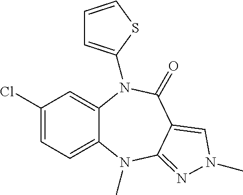 [SMILES: CN1c2ccc(Cl)cc2N(c2cccs2)C(=O)c2cn(C)nc21] | 345.2, 347.2 | 1.94 | 4c |
| 4004 | 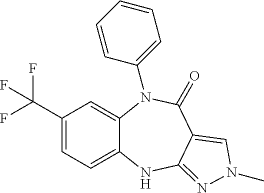 [SMILES: Cn1cc2c(n1)Nc1ccc(C(F)(F)F)cc1N(c1ccccc1)C2=O] | 359.3, 360.4 | 1.86 | 4d |
| 4005 | 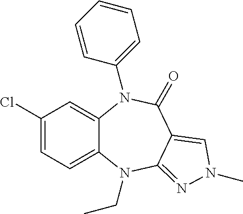 [SMILES: CCN1c2ccc(Cl)cc2N(c2ccccc2)C(=O)c2cn(C)nc21] | 353.3, 355.3 | 2.00 | 4c |
| 4006 | 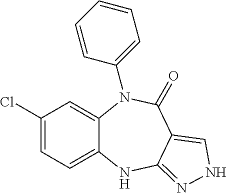 [SMILES: O=C1c2c[nH]nc2Nc2ccc(Cl)cc2N1c1ccccc1] | 3112, 313.2 | 1.80 | 4a |
| 4007 | 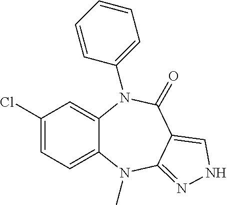 [SMILES: CN1c2ccc(Cl)cc2N(c2ccccc2)C(=O)c2c[nH]nc21] | 325.3, 327.2 | 1.88 | 4i |
| 4008 | 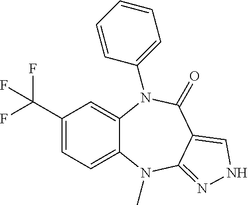 [SMILES: CN1c2ccc(C(F)(F)F)cc2N(c2ccccc2)C(=O)c2c[nH]nc21] | 359.3, 360.4 | 1.90 | 4i |
| 4009 | 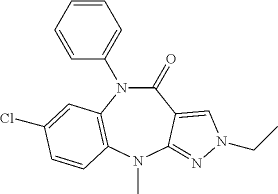 [SMILES: CCn1cc2c(n1)N(C)c1ccc(Cl)cc1N(c1ccccc1)C2=O] | 353.3, 355.3 | 2.01 | 4i |
| 4010 | 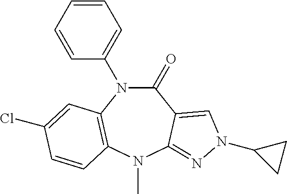 [SMILES: CN1c2ccc(Cl)cc2N(c2ccccc2)C(=O)c2cn(C3CC3)nc21] | 365.3, 367.3 | 2.04 | 4j |
| 4011 | 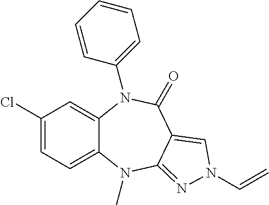 [SMILES: C=Cn1cc2c(n1)N(C)c1ccc(Cl)cc1N(c1ccccc1)C2=O] | 351.3, 352.4 | 2.08 | 4f |
| 4012 | 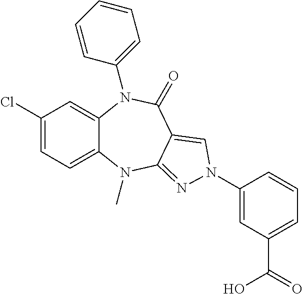 [SMILES: CN1c2ccc(Cl)cc2N(c2ccccc2)C(=O)c2cn(-c3cccc(C(=O)O)c3)nc21] | 445.2, 447.2 | 2.13 | 4i |
| 4013 | 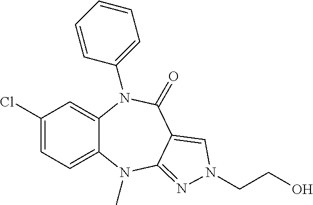 [SMILES: CN1c2ccc(Cl)cc2N(c2ccccc2)C(=O)c2cn(CCO)nc21] | 369.3, 371.2 | 1.85 | 4e |
| 4014 | 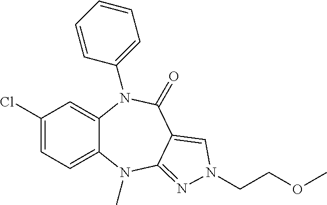 [SMILES: COCCn1cc2c(n1)N(C)c1ccc(Cl)cc1N(c1ccccc1)C2=O] | 383.3, 385.3 | 1.97 | 4h |
| 4015 | 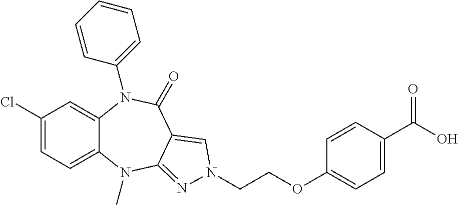 [SMILES: CN1c2ccc(Cl)cc2N(c2ccccc2)C(=O)c2cn(CCOc3ccc(C(=O)O)cc3)nc21] | 489.3, 491.2 | 2.01 | 4i |
| 4016 | 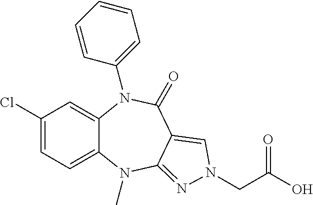 [SMILES: CN1c2ccc(Cl)cc2N(c2ccccc2)C(=O)c2cn(CC(=O)O)nc21] | 383.3, 385.2 | 1.71 | 4h |
| 4017 | 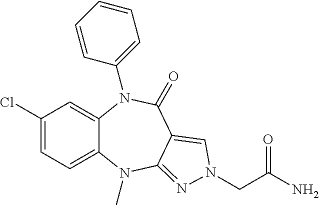 [SMILES: CN1c2ccc(Cl)cc2N(c2ccccc2)C(=O)c2cn(CC(N)=O)nc21] | 382.3, 384.2 | 1.78 | 4h |
| 4018 | 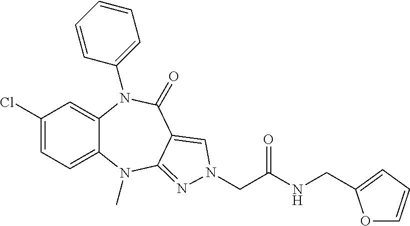 [SMILES: CN1c2ccc(Cl)cc2N(c2ccccc2)C(=O)c2cn(CC(=O)NCc3ccco3)nc21] | 462.3, 464.2 | 1.93 | 4h |
| 4019 | 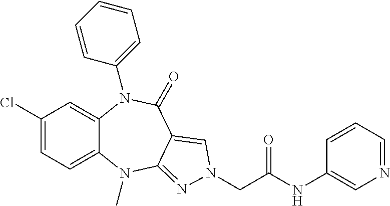 [SMILES: CN1c2ccc(Cl)cc2N(c2ccccc2)C(=O)c2cn(CC(=O)Nc3cccnc3)nc21] | 459.3, 461.2 | 1.90 | 4h |
| 4020 | 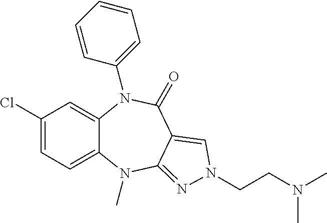 [SMILES: CN(C)CCn1cc2c(n1)N(C)c1ccc(Cl)cc1N(c1ccccc1)C2=O] | 396.3, 398.3 | 1.63 | 4g |
| 4021 | 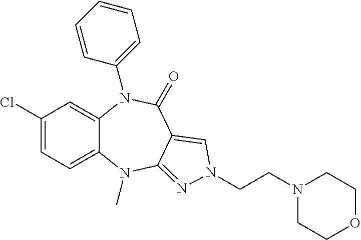 [SMILES: CN1c2ccc(Cl)cc2N(c2ccccc2)C(=O)c2cn(CCN3CCOCC3)nc21] | 438.3, 440.3 | 1.84 | 4g |
| 4022 | 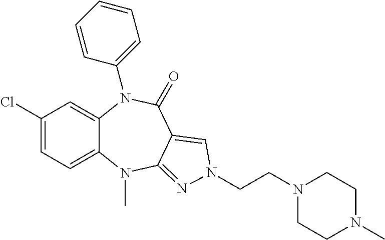 [SMILES: CN1CCN(CCn2cc3c(n2)N(C)c2ccc(Cl)cc2N(c2ccccc2)C3=O)CC1] | 451.4, 453.3 | 1.67 | 4g |
| 4023 | 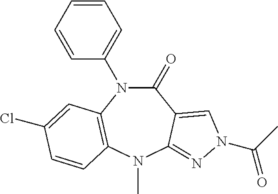 [SMILES: CC(=O)n1cc2c(n1)N(C)c1ccc(Cl)cc1N(c1ccccc1)C2=O] | 367.1, 369.1 | 1.88 | 4i |
| 4024 | 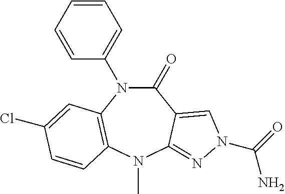 [SMILES: CN1c2ccc(Cl)cc2N(c2ccccc2)C(=O)c2cn(C(N)=O)nc21] | 368.3, 370.2 | 1.91 | 4i |
| 4025 | 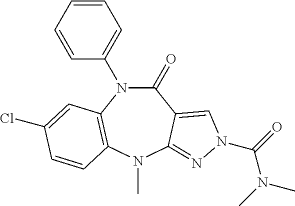 [SMILES: CN(C)C(=O)n1cc2c(n1)N(C)c1ccc(Cl)cc1N(c1ccccc1)C2=O] | 396.3, 398.2 | 1.99 | 4i |
| 4026 | 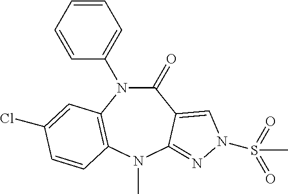 [SMILES: CN1c2ccc(Cl)cc2N(c2ccccc2)C(=O)c2cn(S(C)(=O)=O)nc21] | 403.2, 405.2 | 1.92 | 4i |
| 4027 | 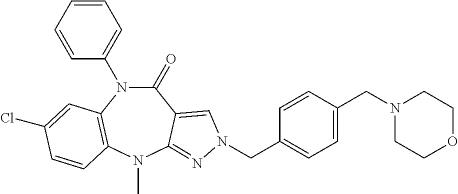 [SMILES: CN1c2ccc(Cl)cc2N(c2ccccc2)C(=O)c2cn(Cc3ccc(CN4CCOCC4)cc3)nc21] | 514.4, 516.3 | 1.80 | 4i |
| 4028 | 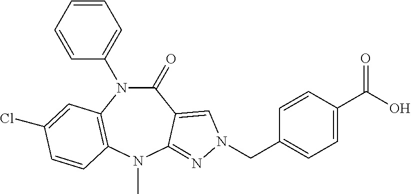 [SMILES: CN1c2ccc(Cl)cc2N(c2ccccc2)C(=O)c2cn(Cc3ccc(C(=O)O)cc3)nc21] | 459.3, 461.2 | 1.99 | 4i |
| 4029 | 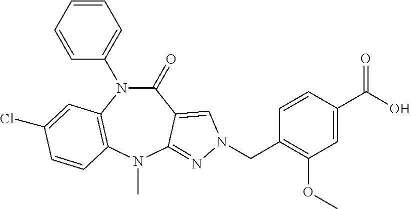 [SMILES: COc1cc(C(=O)O)ccc1Cn1cc2c(n1)N(C)c1ccc(Cl)cc1N(c1ccccc1)C2=O] | 489.3, 491.2 | 2.05 | 4i |
| 4030 | 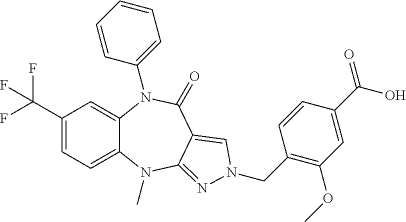 [SMILES: COc1cc(C(=O)O)ccc1Cn1cc2c(n1)N(C)c1ccc(C(F)(F)F)cc1N(c1ccccc1)C2=O] | 523.3 | 2.05 | 4i |
| 4031 | 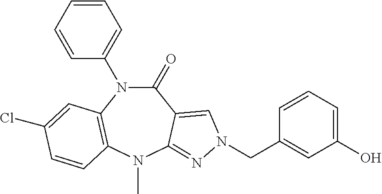 [SMILES: CN1c2ccc(Cl)cc2N(c2ccccc2)C(=O)c2cn(Cc3cccc(O)c3)nc21] | 431.3, 433.2 | 2.02 | 4i |
| 4032 | 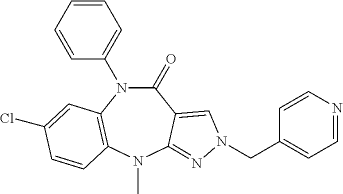 [SMILES: CN1c2ccc(Cl)cc2N(c2ccccc2)C(=O)c2cn(Cc3ccncc3)nc21] | 416.3, 418.3 | 1.93 | 4i |
| 4033 | 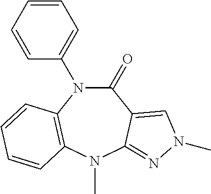 [SMILES: CN1c2ccccc2N(c2ccccc2)C(=O)c2cn(C)nc21] | 305.3, 306.4 | 1.77 | 4e |
| 4034 | 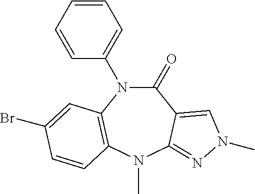 [SMILES: CN1c2ccc(Br)cc2N(c2ccccc2)C(=O)c2cn(C)nc21] | 385.2, 386.3 | 1.95 | 4e |
| 4035 | 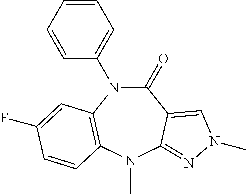 [SMILES: CN1c2ccc(F)cc2N(c2ccccc2)C(=O)c2cn(C)nc21] | 323.3, 324.4 | 1.81 | 4e |
| 4036 | 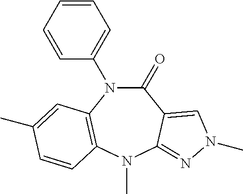 | 319.3 | 1.87 | 4e |
| 4037 | 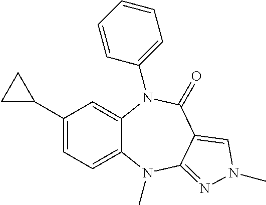 [SMILES: CN1c2ccc(C3CC3)cc2N(c2ccccc2)C(=O)c2cn(C)nc21] | 345.4 | 1.98 | 4e |
| 4038 | 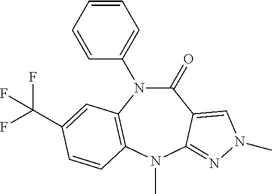 [SMILES: CN1c2ccc(C(F)(F)F)cc2N(c2ccccc2)C(=O)c2cn(C)nc21] | 373.3 | 1.94 | 4e |
| 4039 | 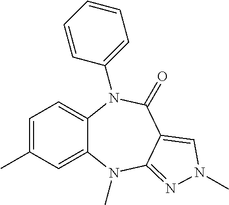 | 319.1 | 5.40^{§} | 4e |
| 4040 | 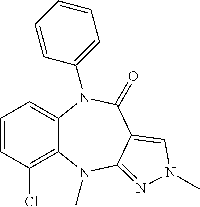 [SMILES: CN1c2nn(C)cc2C(=O)N(c2ccccc2)c2cccc(Cl)c21] | 339.1, 341.1 | 5.50^{§} | 4e |
| 4041 | 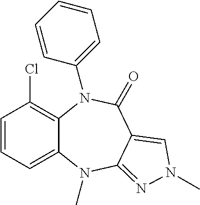 [SMILES: CN1c2cccc(Cl)c2N(c2ccccc2)C(=O)c2cn(C)nc21] | 339.1, 341.1 | 5.58^{§} | 4e |

TABLE 5

| | | | t_{R} | |
|---|---|---|---|---|
| Cmpd # | Structure | (M + H)⁺ | (min) | Example |
| 5001 | 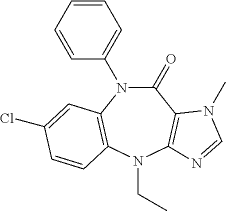 [SMILES: CCN1c2ccc(Cl)cc2N(c2ccccc2)C(=O)c2c1ncn2C] | 353.1, 355.1 | 2.02 | 5a |
| 5002 | 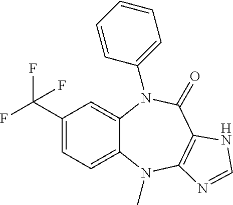 [SMILES: CN1c2ccc(C(F)(F)F)cc2N(c2ccccc2)C(=O)c2[nH]cnc21] | 359.1 | 1.88 | 5a |
| 5003 | 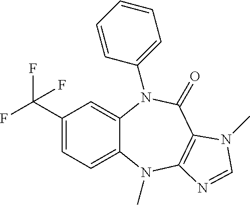 [SMILES: CN1c2ccc(C(F)(F)F)cc2N(c2ccccc2)C(=O)c2c1ncn2C] | 373.1 | 1.96 | 5a |
| 5004 | 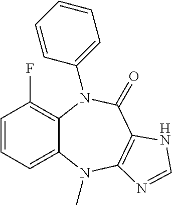 [SMILES: CN1c2cccc(F)c2N(c2ccccc2)C(=O)c2[nH]cnc21] | 309.1 | 1.75 | 5a |
| 5005 | 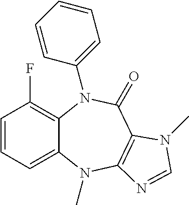 [SMILES: CN1c2cccc(F)c2N(c2ccccc2)C(=O)c2c1ncn2C] | 323.0 | 4.86^{Δ} | 5a |
| 5006 | 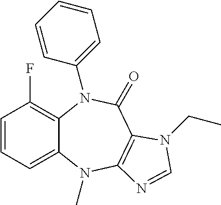 [SMILES: CCn1cnc2c1C(=O)N(c1ccccc1)c1c(F)cccc1N2C] | 337.0 | 1.88 | 5a |
| 5007 | 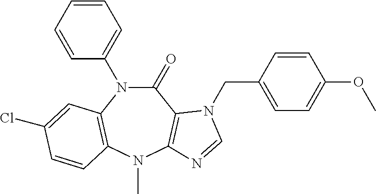 [SMILES: COc1ccc(Cn2cnc3c2C(=O)N(c2ccccc2)c2cc(Cl)ccc2N3C)cc1] | 445.1, 447.1 | 2.15 | 5a |
| 5008 | 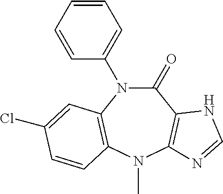 [SMILES: CN1c2ccc(Cl)cc2N(c2ccccc2)C(=O)c2[nH]cnc21] | 325.0, 327.0 | 1.87 | 5a |
| 5009 | 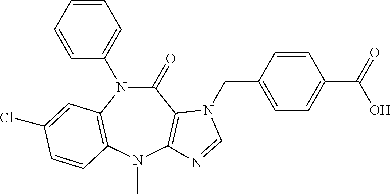 [SMILES: CN1c2ccc(Cl)cc2N(c2ccccc2)C(=O)c2c1ncn2Cc1ccc(C(=O)O)cc1] | 458.9, 460.9 | 5.21^{§} | 5d |
| 5010 | 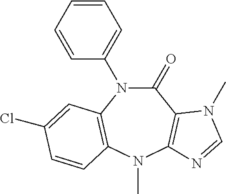 [SMILES: CN1c2ccc(Cl)cc2N(c2ccccc2)C(=O)c2c1ncn2C] | 339.1, 341.1 | 1.95 | 5a |
| 5011 | 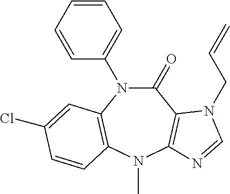 [SMILES: C=CCn1cnc2c1C(=O)N(c1ccccc1)c1cc(Cl)ccc1N2C] | 364.9, 366.9 | 2.0 | 5a |
| 5012 | 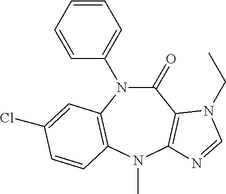 [SMILES: CCn1cnc2c1C(=O)N(c1ccccc1)c1cc(Cl)ccc1N2C] | 352.8, 354.8 | 2.02 | 5a |
| 5013 | 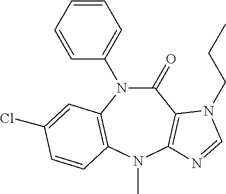 [SMILES: CCCn1cnc2c1C(=O)N(c1ccccc1)c1cc(Cl)ccc1N2C] | 367.1, 369.1 | 2.09 | 5a |
| 5014 | 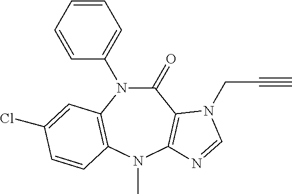 [SMILES: C#CCn1cnc2c1C(=O)N(c1ccccc1)c1cc(Cl)ccc1N2C] | 363.1, 365.1 | 1.97 | 5a |
| 5015 | 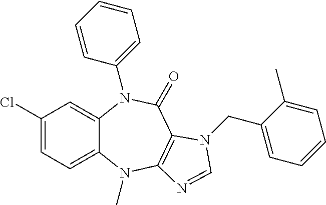 [SMILES: Cc1ccccc1Cn1cnc2c1C(=O)N(c1ccccc1)c1cc(Cl)ccc1N2C] | 429.1, 431.1 | 2.21 | 5a |
| 5016 | 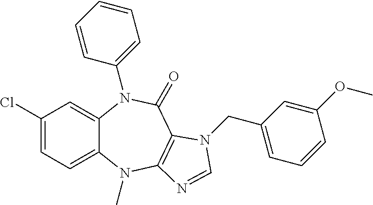 [SMILES: COc1cccc(Cn2cnc3c2C(=O)N(c2ccccc2)c2cc(Cl)ccc2N3C)c1] | 445.2, 447.1 | 2.15 | 5a |
| 5017 | 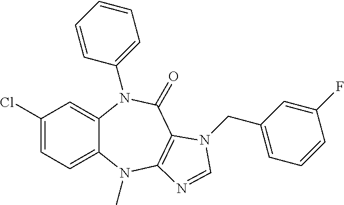 [SMILES: CN1c2ccc(Cl)cc2N(c2ccccc2)C(=O)c2c1ncn2Cc1cccc(F)c1] | 433.1, 435.1 | 2.15 | 5a |
| 5018 | 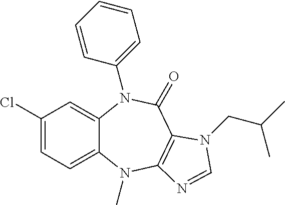 [SMILES: CC(C)Cn1cnc2c1C(=O)N(c1ccccc1)c1cc(Cl)ccc1N2C] | 381.1, 383.1 | 2.15 | 5a |
| 5019 | 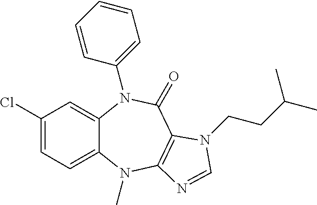 [SMILES: CC(C)CCn1cnc2c1C(=O)N(c1ccccc1)c1cc(Cl)ccc1N2C] | 395.7, 397.0 | 2.24 | 5a |
| 5020 | 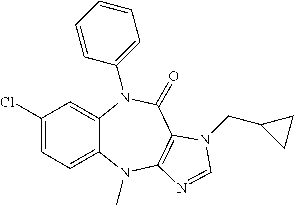 [SMILES: CN1c2ccc(Cl)cc2N(c2ccccc2)C(=O)c2c1ncn2CC1CC1] | 379.1, 381.1 | 2.10 | 5a |
| 5021 | 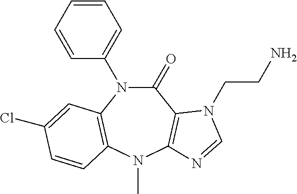 [SMILES: CN1c2ccc(Cl)cc2N(c2ccccc2)C(=O)c2c1ncn2CCN] | 368.1, 370.1 | 1.65 | 5f |
| 5022 | 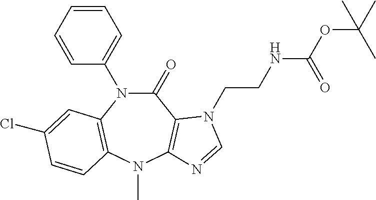 [SMILES: CN1c2ccc(Cl)cc2N(c2ccccc2)C(=O)c2c1ncn2CCNC(=O)OC(C)(C)C] | 468.2, 470.1 | 2.07 | 5a |
| 5023 | 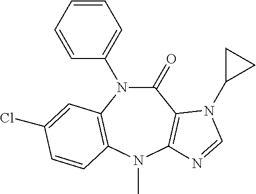 [SMILES: CN1c2ccc(Cl)cc2N(c2ccccc2)C(=O)c2c1ncn2C1CC1] | 365.1, 367.1 | 2.03 | 5e |
| 5024 | 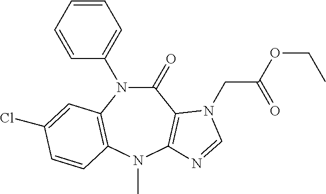 [SMILES: CCOC(=O)Cn1cnc2c1C(=O)N(c1ccccc1)c1cc(Cl)ccc1N2C] | 411.1, 413.0 | 1.98 | 5a |
| 5025 | 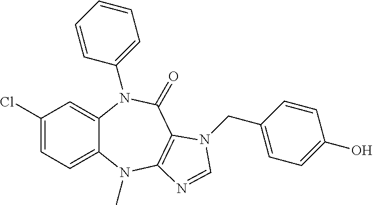 [SMILES: CN1c2ccc(Cl)cc2N(c2ccccc2)C(=O)c2c1ncn2Cc1ccc(O)cc1] | 431.1, 433.1 | 1.97 | 5c |
| 5026 | 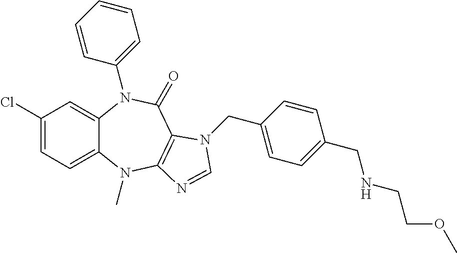 [SMILES: COCCNCc1ccc(Cn2cnc3c2C(=O)N(c2ccccc2)c2cc(Cl)ccc2N3C)cc1] | 502.1, 504.1 | 5.20^{§} | 5b |
| 5027 | 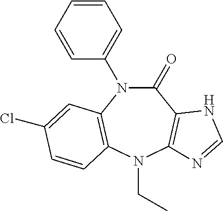 [SMILES: CCN1c2ccc(Cl)cc2N(c2ccccc2)C(=O)c2[nH]cnc21] | 339.1, 341.1 | 1.97 | 5a |
| 5028 | 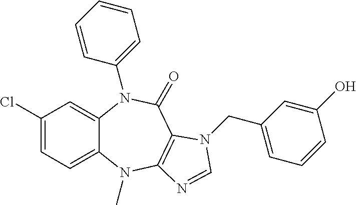 [SMILES: CN1c2ccc(Cl)cc2N(c2ccccc2)C(=O)c2c1ncn2Cc1cccc(O)c1] | 431.1, 433.0 | 1.99 | 5c |
| 5029 | 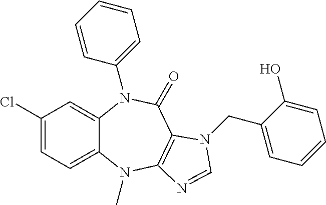 [SMILES: CN1c2ccc(Cl)cc2N(c2ccccc2)C(=O)c2c1ncn2Cc1ccccc1O] | 431.0, 433.0 | 5.56^{§} | 5c |
| 5030 | 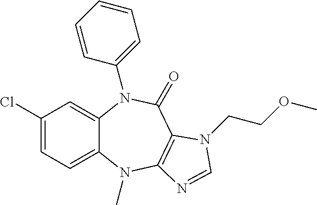 [SMILES: COCCn1cnc2c1C(=O)N(c1ccccc1)c1cc(Cl)ccc1N2C] | 383.1, 385.1 | 1.98 | 5a |
| 5031 | 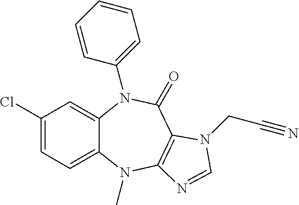 [SMILES: CN1c2ccc(Cl)cc2N(c2ccccc2)C(=O)c2c1ncn2CC#N] | 364.1, 366.1 | 1.88 | 5a |

TABLE 6

| | | | t_{R} | |
|---|---|---|---|---|
| Cmpd # | Structure | (M + H)⁺ | (min) | Example |
| 6001 | 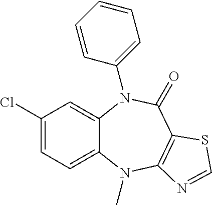 [SMILES: CN1c2ccc(Cl)cc2N(c2ccccc2)C(=O)c2scnc21] | 342.0, 344.0 | 2.00 | 6b |
| 6002 | 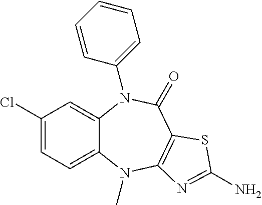 [SMILES: CN1c2ccc(Cl)cc2N(c2ccccc2)C(=O)c2sc(N)nc21] | 357.0, 359.0 | 1.90 | 6a |
| 6003 | 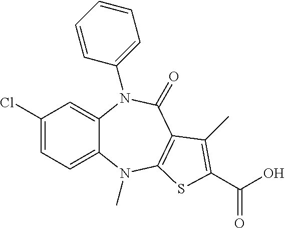 [SMILES: Cc1c(C(=O)O)sc2c1C(=O)N(c1ccccc1)c1cc(Cl)ccc1N2C] | 399.0, 401.0 | 1.99 | 6c |
| 6004 | 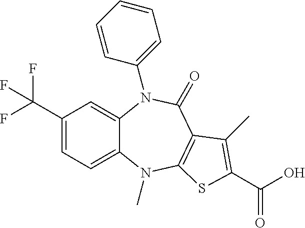 [SMILES: Cc1c(C(=O)O)sc2c1C(=O)N(c1ccccc1)c1cc(C(F)(F)F)ccc1N2C] | 433.0 | 1.98 | 6c |
| 6005 | 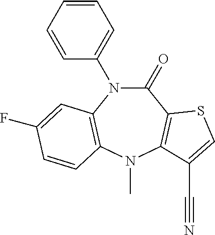 [SMILES: CN1c2ccc(F)cc2N(c2ccccc2)C(=O)c2scc(C#N)c21] | 350.0 | 1.92 | 6d |
| 6006 | 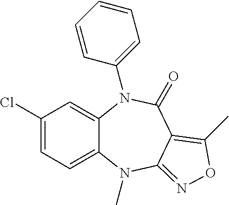 [SMILES: Cc1onc2c1C(=O)N(c1ccccc1)c1cc(Cl)ccc1N2C] | 340.0, 342.0 | 2.03 | 6d |
| 6007 | 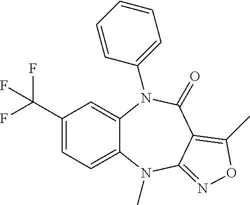 [SMILES: Cc1onc2c1C(=O)N(c1ccccc1)c1cc(C(F)(F)F)ccc1N2C] | 374.1 | 2.03 | 6d |
| 6008 | 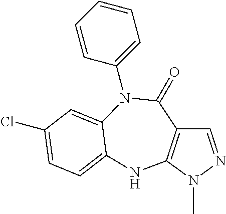 [SMILES: Cn1ncc2c1Nc1ccc(Cl)cc1N(c1ccccc1)C2=O] | 325.3, 327.3 | 6.38^{§} | 4a |

TABLE 7

| | | | t_{R} | |
|---|---|---|---|---|
| Cmpd # | Structure | (M + H)⁺ | (min) | Example |
| 7001 | 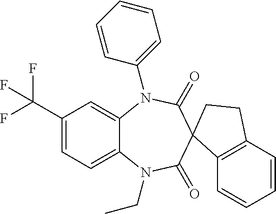 [SMILES: CCN1C(=O)C2(CCc3ccccc32)C(=O)N(c2ccccc2)c2cc(C(F)(F)F)ccc21] | 451.4 | 1.98 | 7a |
| 7002 | 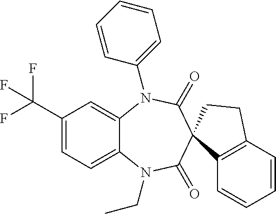 [SMILES: CCN1C(=O)[C@@]2(CCc3ccccc32)C(=O)N(c2ccccc2)c2cc(C(F)(F)F)ccc21] | 451.3 | 1.98 | 7b |
| 7003 | 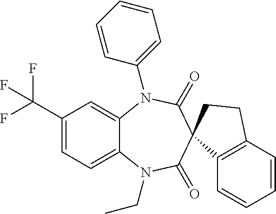 [SMILES: CCN1C(=O)[C@]2(CCc3ccccc32)C(=O)N(c2ccccc2)c2cc(C(F)(F)F)ccc21] | 451.4 | 1.98 | 7b |
| 7004 | 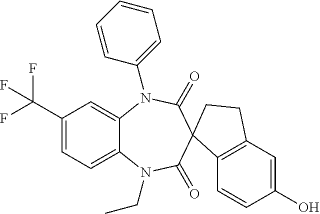 [SMILES: CCN1C(=O)C2(CCc3cc(O)ccc32)C(=O)N(c2ccccc2)c2cc(C(F)(F)F)ccc21] | 467.3 | 1.84 | 7d |
| 7005 | 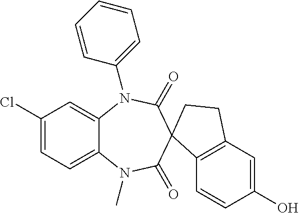 [SMILES: CN1C(=O)C2(CCc3cc(O)ccc32)C(=O)N(c2ccccc2)c2cc(Cl)ccc21] | 419.3, 421.2 | 1.77 | 7c |
| 7006 | 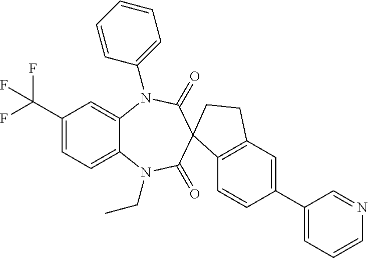 [SMILES: CCN1C(=O)C2(CCc3cc(-c4cccnc4)ccc32)C(=O)N(c2ccccc2)c2cc(C(F)(F)F)ccc21] | 528.3 | 1.98 | 7d |
| 7007 | 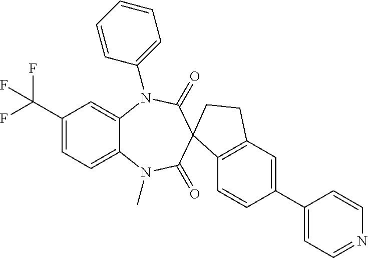 [SMILES: CN1C(=O)C2(CCc3cc(-c4ccncc4)ccc32)C(=O)N(c2ccccc2)c2cc(C(F)(F)F)ccc21] | 514.2 | 5.33^{§} | 7d |
| 7008 | 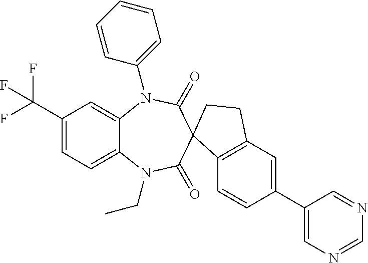 [SMILES: CCN1C(=O)C2(CCc3cc(-c4cncnc4)ccc32)C(=O)N(c2ccccc2)c2cc(C(F)(F)F)ccc21] | 529.4 | 1.93 | 7d |
| 7009 | 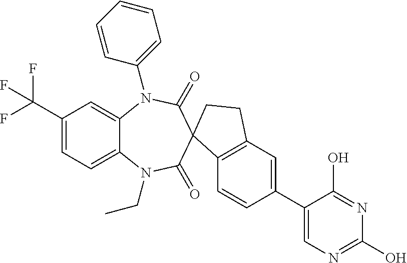 [SMILES: CCN1C(=O)C2(CCc3cc(-c4cnc(O)nc4O)ccc32)C(=O)N(c2ccccc2)c2cc(C(F)(F)F)ccc21] | 561.3 | 1.82 | 7d |
| 7010 | 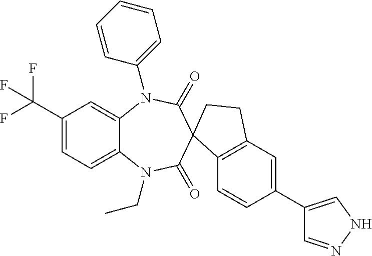 [SMILES: CCN1C(=O)C2(CCc3cc(-c4cn[nH]c4)ccc32)C(=O)N(c2ccccc2)c2cc(C(F)(F)F)ccc21] | 517.2 | 1.91 | 7d |
| 7011 | 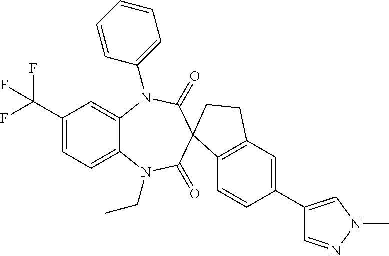 [SMILES: CCN1C(=O)C2(CCc3cc(-c4cnn(C)c4)ccc32)C(=O)N(c2ccccc2)c2cc(C(F)(F)F)ccc21] | 531.3 | 1.94 | 7d |
| 7012 | 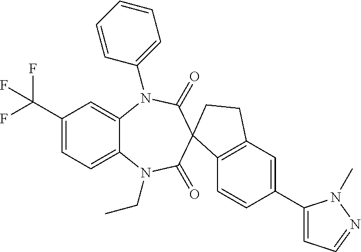 [SMILES: CCN1C(=O)C2(CCc3cc(-c4ccnn4C)ccc32)C(=O)N(c2ccccc2)c2cc(C(F)(F)F)ccc21] | 531.4 | 1.99 | 7d |
| 7013 | 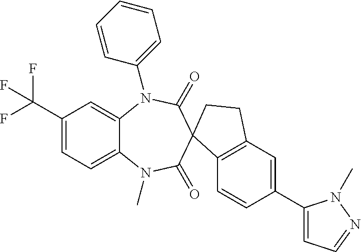 [SMILES: CN1C(=O)C2(CCc3cc(-c4ccnn4C)ccc32)C(=O)N(c2ccccc2)c2cc(C(F)(F)F)ccc21] | 517.4 | 1.94 | 7d |
| 7014 | 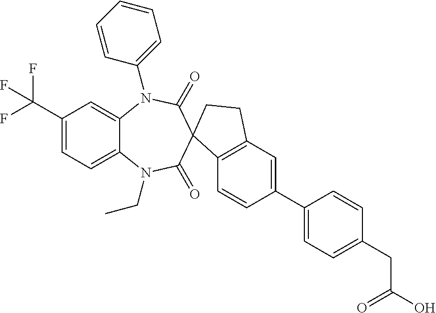 [SMILES: CCN1C(=O)C2(CCc3cc(-c4ccc(CC(=O)O)cc4)ccc32)C(=O)N(c2ccccc2)c2cc(C(F)(F)F)ccc21] | 585.4 | 2.04 | 7d |
| 7015 | 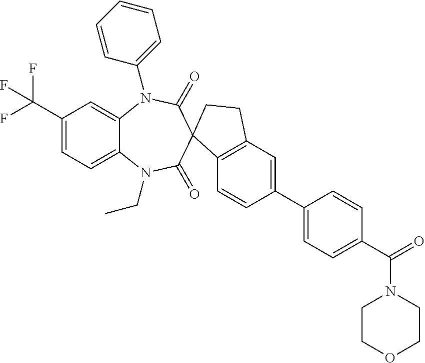 [SMILES: CCN1C(=O)C2(CCc3cc(-c4ccc(C(=O)N5CCOCC5)cc4)ccc32)C(=O)N(c2ccccc2)c2cc(C(F)(F)F)ccc21] | 640.4 | 2.00 | 7d |
| 7016 | 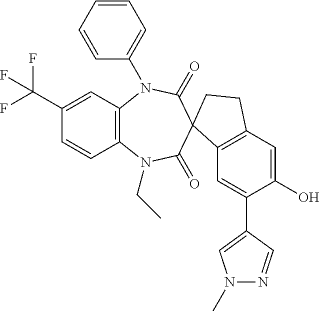 [SMILES: CCN1C(=O)C2(CCc3cc(O)c(-c4cnn(C)c4)cc32)C(=O)N(c2ccccc2)c2cc(C(F)(F)F)ccc21] | 547.4 | 1.85 | 7c, 7g |
| 7017 | 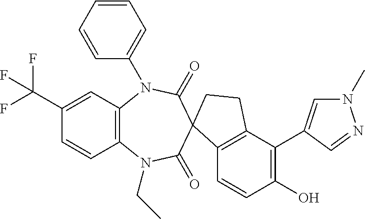 [SMILES: CCN1C(=O)C2(CCc3c2ccc(O)c3-c2cnn(C)c2)C(=O)N(c2ccccc2)c2cc(C(F)(F)F)ccc21] | 547.4 | 1.84 | 7c, 7g |
| 7018 | 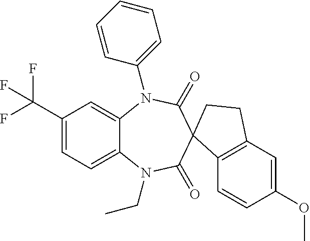 [SMILES: CCN1C(=O)C2(CCc3cc(OC)ccc32)C(=O)N(c2ccccc2)c2cc(C(F)(F)F)ccc21] | 481.3 | 1.97 | 7c |
| 7019 | 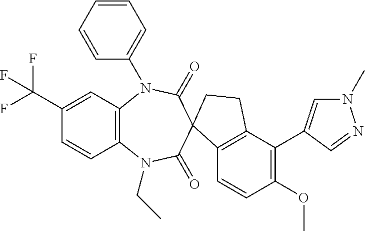 [SMILES: CCN1C(=O)C2(CCc3c2ccc(OC)c3-c2cnn(C)c2)C(=O)N(c2ccccc2)c2cc(C(F)(F)F)ccc21] | 561.4 | 1.92 | 7g |
| 7020 | 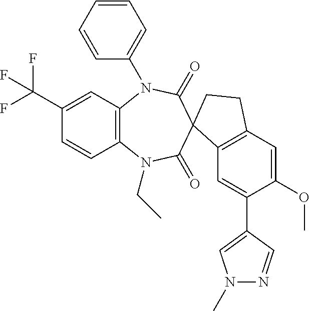 [SMILES: CCN1C(=O)C2(CCc3cc(OC)c(-c4cnn(C)c4)cc32)C(=O)N(c2ccccc2)c2cc(C(F)(F)F)ccc21] | 561.1 | 6.85^{§} | 7g |
| 7021 | 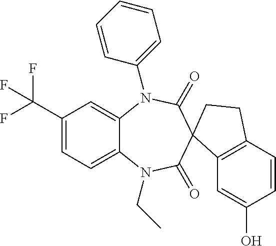 [SMILES: CCN1C(=O)C2(CCc3ccc(O)cc32)C(=O)N(c2ccccc2)c2cc(C(F)(F)F)ccc21] | 467.3 | 1.88 | 7c |
| 7022 | 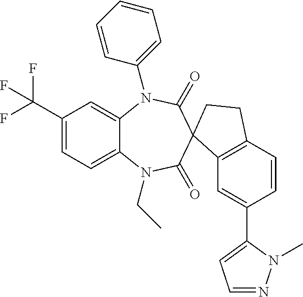 [SMILES: CCN1C(=O)C2(CCc3ccc(-c4ccnn4C)cc32)C(=O)N(c2ccccc2)c2cc(C(F)(F)F)ccc21] | 531.4 | 2.01 | 7d |
| 7023 | 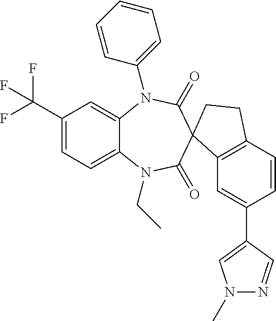 [SMILES: CCN1C(=O)C2(CCc3ccc(-c4cnn(C)c4)cc32)C(=O)N(c2ccccc2)c2cc(C(F)(F)F)ccc21] | 531.1 | 6.46^{§} | 7d |
| 7024 | 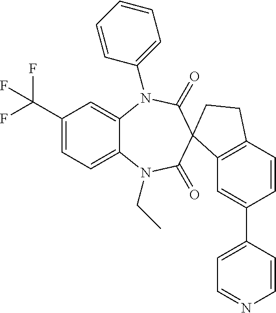 [SMILES: CCN1C(=O)C2(CCc3ccc(-c4ccncc4)cc32)C(=O)N(c2ccccc2)c2cc(C(F)(F)F)ccc21] | 528.1 | 5.49^{§} | 7d |
| 7025 | 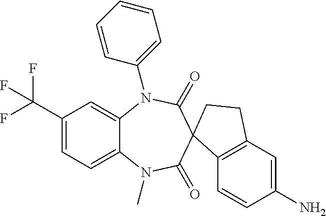 [SMILES: CN1C(=O)C2(CCc3cc(N)ccc32)C(=O)N(c2ccccc2)c2cc(C(F)(F)F)ccc21] | 452.3 | 1.71 | 7h |
| 7026 | 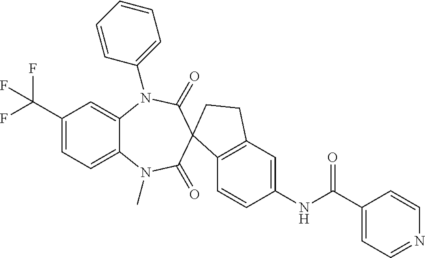 [SMILES: CN1C(=O)C2(CCc3cc(NC(=O)c4ccncc4)ccc32)C(=O)N(c2ccccc2)c2cc(C(F)(F)F)ccc21] | 557.3 | 1.86 | 7h |
| 7027 | 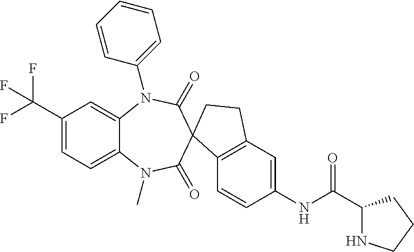 [SMILES: CN1C(=O)C2(CCc3cc(NC(=O)[C@@H]4CCCN4)ccc32)C(=O)N(c2ccccc2)c2cc(C(F)(F)F)ccc21] | 549.4 | 1.65 | 7h |
| 7028 | 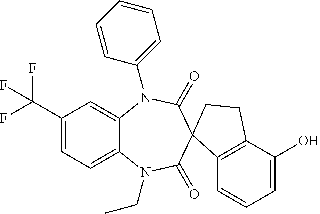 [SMILES: CCN1C(=O)C2(CCc3c(O)cccc32)C(=O)N(c2ccccc2)c2cc(C(F)(F)F)ccc21] | 467.3 | 1.83 | 7c |
| 7029 | 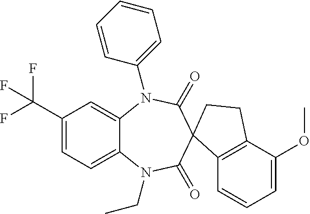 [SMILES: CCN1C(=O)C2(CCc3c(OC)cccc32)C(=O)N(c2ccccc2)c2cc(C(F)(F)F)ccc21] | 481.4 | 2.00 | 7g |
| 7030 | 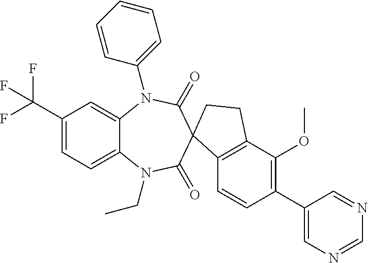 [SMILES: CCN1C(=O)C2(CCc3c2ccc(-c2cncnc2)c3OC)C(=O)N(c2ccccc2)c2cc(C(F)(F)F)ccc21] | 559.4 | 1.93 | 7g |
| 7031 | 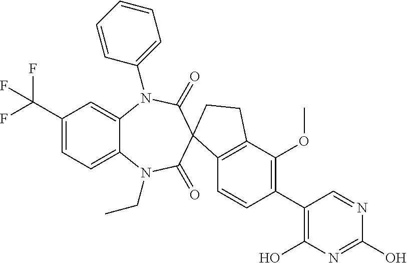 [SMILES: CCN1C(=O)C2(CCc3c2ccc(-c2cnc(O)nc2O)c3OC)C(=O)N(c2ccccc2)c2cc(C(F)(F)F)ccc21] | 591.3 | 1.80 | 7g |
| 7032 | 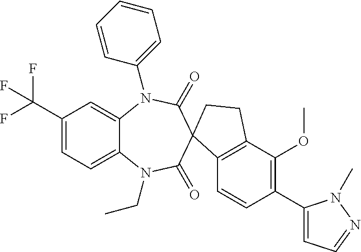 [SMILES: CCN1C(=O)C2(CCc3c2ccc(-c2ccnn2C)c3OC)C(=O)N(c2ccccc2)c2cc(C(F)(F)F)ccc21] | 561.4 | 1.98 | 7g |
| 7033 | 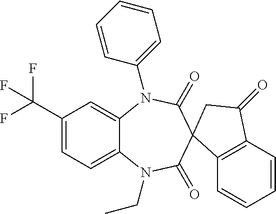 [SMILES: CCN1C(=O)C2(CC(=O)c3ccccc32)C(=O)N(c2ccccc2)c2cc(C(F)(F)F)ccc21] | 465.3 | 1.85 | 7e |
| 7034 | 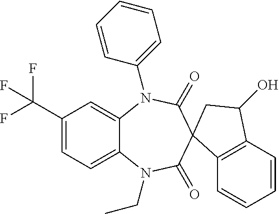 [SMILES: CCN1C(=O)C2(CC(O)c3ccccc32)C(=O)N(c2ccccc2)c2cc(C(F)(F)F)ccc21] | 449.3, 467.3 | 1.81 | 7e |
| 7035 | 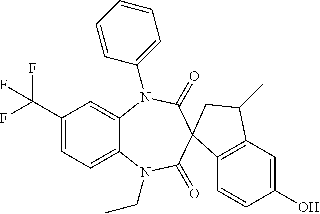 [SMILES: CCN1C(=O)C2(CC(C)c3cc(O)ccc32)C(=O)N(c2ccccc2)c2cc(C(F)(F)F)ccc21] | 481.3 | 1.90 | 7f |
| 7036 | 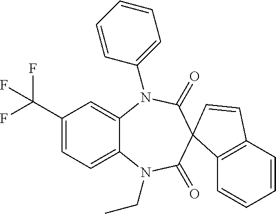 [SMILES: CCN1C(=O)C2(C=Cc3ccccc32)C(=O)N(c2ccccc2)c2cc(C(F)(F)F)ccc21] | 449.3 | 1.99 | 7e |
| 7037 | 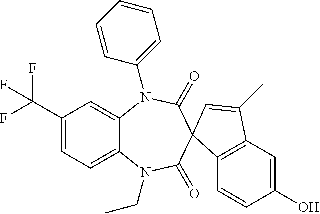 [SMILES: CCN1C(=O)C2(C=C(C)c3cc(O)ccc32)C(=O)N(c2ccccc2)c2cc(C(F)(F)F)ccc21] | 479 | 6.38^{§} | 7e |

Each reference, including all patents, patent applications, and publications cited in the present application is incorporated herein by reference in its entirety, as if each of them is individually incorporated. Further, it would be appreciated that, in the above teaching of invention, the skilled in the art could make certain changes or modifications to the invention, and these equivalents would still be within the scope of the invention defined by the appended claims of the application.

1. A compound of formula (I) or salt thereof: 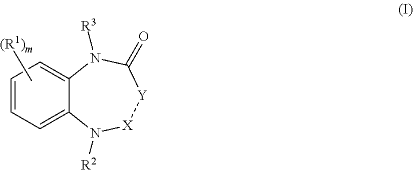
wherein
m is 1, 2 or 3;
either X---Y is selected from: 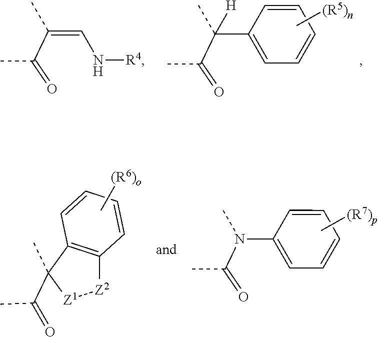
or X and Y are linked to form a 5-membered heteroaryl ring containing 1 to 3 heteroatoms independently selected from O, N and S, wherein said 5-membered heteroaryl ring is optionally substituted 1 to 2 times with substituents independently selected from (C₁₋₆)alkyl, (C₂₋₆)alkenyl, (C₂₋₆)alkynyl, (C₃₋₇)cycloalkyl, —(C₁₋₆)alkyl-(C₃₋₇)cycloalkyl, —(C₁₋₆)alkyl-Het,
—(C₁₋₆)alkyl-aryl, aryl, —NH₂, COON, CN, —C(═O)—(C₁₋₆)alkyl, —C(═O)—NH₂, —C(═O)—N((C₁₋₆)alkyl)₂ and —SO₂—(C₁₋₆)alkyl;
wherein each said alkyl, aryl and Het, either alone or in combination with another radical is optionally substituted 1 to 2 times with substituents independently selected from (C₁₋₆)alkyl, —O—(C₁₋₆)alkyl, OH, CN, —COON, halo, —(C₁₋₆)alkyl-Het, —(C₁₋₆)alkyl-aryl, —NH₂, —NH(C₁₋₆)alkyl, —N((C₁₋₆)alkyl)₂, —N(H)—C(═O)—O—(C₁₋₆)alkyl, —(C₁₋₆)alkyl-N(H)—(C₁₋₆)alkyl-O—(C₁₋₆)alkyl, —O-aryl-C(═O)OH, —C(═O)—O—(C₁₋₆)alkyl, —C(═O)NH₂, —C(═O)—N(H)—(C₁₋₆)alkyl-Het, —C(═O)—N(H)-Het and Het;
R¹ is independently selected from (C₁₋₆)alkyl, (C₁₋₆)haloalkyl, halo and (C₃₋₇)cycloalkyl;
R² is H, (C₁₋₆)alkyl, —(C₁₋₆)alkyl-(C₃₋₇)cycloalkyl or (C₃₋₇)cycloalkyl;
R³ is phenyl or thiophene, wherein said phenyl is optionally substituted in the meta- or para-position with (C₁₋₆)alkyl, halo, CN, OH or —O—(C₁₋₆)alkyl;
R⁴ is (C₁₋₆)alkyl optionally substituted 1 or 2 times with —O—(C₁₋₆)alkyl, Het, —COOH or OH;
R⁵ and R⁷ are each independently selected from OH, CN, halo, —COON, R⁵¹, —O—R⁵¹, —S—R⁵¹, —SO—R⁵¹, —SO₂—R⁵¹, —C(═O)—NH₂, —C(═O)—N(R⁵¹)(R⁵²), —(C₁₋₆)alkyl-NH(R⁵¹), —(C₁₋₆)alkyl-O—R⁵¹ and —(C₁₋₆)alkyl-S—R⁵¹;
R⁵¹ is selected from (C₁₋₆)alkyl, aryl, —(C₁₋₆)alkyl-aryl, Het and —(C₁₋₆)alkyl-Het;
wherein said aryl and Het are optionally fused to ring D;
wherein each said alkyl is optionally substituted 1 or 2 times with substituents independently selected from R⁵³;
wherein each said aryl and Het are optionally substituted 1 to 2 times with substituents independently selected from R⁵³, —O—(C₁₋₆)alkyl, —OH, oxo, —C(═O)O(C₁₋₆)alkyl, —C(═O)—Het and (C₁₋₆)alkyl optionally substituted one time with Het or R⁵³;
R⁵³ is —COOH, —NH₂, —NH(C₁₋₆)alkyl, —N((C₁₋₆)alkyl)₂, —O—(C₁₋₆)alkyl or —OH;
R⁵² is selected from H and (C₁₋₆)alkyl;
Z¹ and Z² are either both defined as CH₂ and ---- is a single bond or Z¹ and Z² are both defined as CH and ---- is a double bond;
n, o and p are each independently selected from 0, 1, 2 or 3;
R⁶ is selected from (C₁₋₆)alkyl, —O—(C₁₋₆)alkyl, OH, NH₂, —N(H)(C₁₋₆)alkyl, —N((C₁₋₆)alkyl)₂, —N(H)—(C₁₋₆)alkyl-aryl, —N(H)—(C₁₋₆)alkyl-Het, —N(H)—C(═O)-aryl, —N(H)—C(═O)-Het, —N(H)—C(═O)—NH₂, oxo, Het and aryl,
wherein each said aryl and Het are optionally substituted 1 to 2 times with substituents independently selected from R⁶¹, —O—(C₁₋₆)alkyl, —OH, —C(═O)—Het and (C₁₋₆)alkyl optionally substituted one time with R⁶¹;
R⁶¹ is —COON, —N((C₁₋₆)alkyl)₂ or —OH;
with the proviso that the following compounds are excluded:
8-chloro-3-(methylamino-methylene)-1-phenyl-1,5-dihydro-benzo[b][1,4]diazepine-2,4-dione;
3-(butylamino-methylene)-8-chloro-1-phenyl-1,5-dihydro-benzo[b][1,4]diazepine-2,4-dione;
3-(tert-butylamino-methylene)-8-chloro-1-phenyl-1,5-dihydro-benzo[b][1,4]diazepine-2,4-dione;
8-chloro-3-(isobutylamino-methylene)-1-phenyl-1,5-dihydro-benzo[b][1,4]diazepine-2,4-dione;
8-bromo-3-(butylamino-methylene)-1-phenyl-1,5-dihydro-benzo[b][1,4]diazepine-2,4-dione; and
3-(butylamino-methylene)-1-phenyl-8-trifluoromethyl-1,5-dihydro-benzo[b][1,4]diazepine-2,4-dione.

2. The compound according to claim 1, or a pharmaceutically acceptable salt thereof, wherein R³ is phenyl.

3. The compound according to claim 1, or a pharmaceutically acceptable salt thereof, wherein R³ is thiophene.

4. The compound according to claim 1, or a pharmaceutically acceptable salt thereof, wherein m is 1 and R¹ is selected from Cl, F, CH₃ and CF₃.

5. The compound according to claim 4, or a pharmaceutically acceptable salt thereof, wherein R¹ is CF₃.

6. The compound according to claim 1, or a pharmaceutically acceptable salt thereof, wherein R² is H or (C₁₋₆)alkyl.

7. The compound according to claim 1, or a pharmaceutically acceptable salt thereof, wherein X and Y are 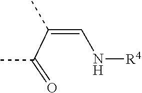
, and
R⁴ is (C₁₋₄)alkyl optionally substituted one time with —O—(C₁₋₄)alkyl, 5-membered Het, COON or OH.

8. The compound according to claim 1, or a pharmaceutically acceptable salt thereof, wherein X and Y are 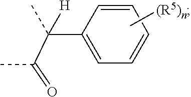
R⁵ is selected from OH, CN, halo, —COOH, R⁵¹, —O—R⁵¹, —S—R⁵¹, —SO—R⁵¹, —SO₂—R⁵¹ and —C(═O)—NH₂;
R⁵¹ is selected from (C₁₋₃)alkyl, Het and —(C₁₋₃)alkyl-Het;
wherein said Het is optionally fused to ring D;
wherein each said alkyl is optionally substituted 1 or 2 times with substituents independently selected from R⁵³;
wherein each said phenyl and Het are optionally substituted 1 to 2 times with substituents independently selected from R⁵³, —O—(C₁₋₃)alkyl, —OH, oxo and (C₁₋₃)alkyl optionally substituted one time with Het or R⁵³;
R⁵³ is —COON, —N((C₁₋₃)alkyl)₂, —O—(C₁₋₃)alkyl or —OH; and
Het is defined as a saturated or unsaturated 5- or 6-membered monocyclic ring system, including an aromatic ring system, containing one heteroatom selected from N, O or S; and
n is 0 or 1.

9. The compound according to claim 1, or a pharmaceutically acceptable salt thereof, wherein X and Y are 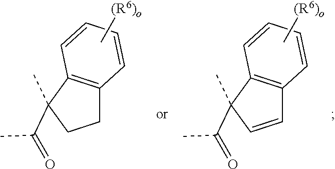
R⁶ is selected from (C₁₋₃)alkyl, —O—(C₁₋₃)alkyl, OH, NH₂, —N(H)(C₁₋₆)alkyl, —N((C₁₋₆)alkyl)₂, —N(H)—C(═O)-phenyl, —N(H)—C(═O)—Het, oxo, Het and phenyl,
wherein each said phenyl and Het are optionally substituted 1 to 2 times with substituents independently selected from R⁶¹, —O—(C₁₋₃)alkyl, —OH, —C(═O)—Het and (C₁₋₃)alkyl optionally substituted one time with R⁶¹;
R⁶¹ is —COON, —N((C₁₋₃)alkyl)₂ or —OH; and
Het is defined as a saturated or unsaturated 5- or 6-membered monocyclic ring system, including an aromatic ring system, containing 1 to 3 heteroatoms selected from N, O or S; and
o is 0 or 1.

10. The compound according to claim 1, or a pharmaceutically acceptable salt thereof, wherein X and Y are 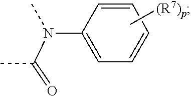
R⁷ is selected from OH, halo, —COON, R⁵¹, —O—R⁵¹, C(═O)—NH(R⁵¹), —(C₁₋₃)alkyl-NH(R⁵¹), —(C₁₋₃)alkyl-O—R⁵¹ and —(C₁₋₃)alkyl-S—R⁵¹;
R⁵¹ is selected from (C₁₋₃)alkyl, phenyl, —(C₁₋₃)alkyl-phenyl, Het and —(C₁₋₃)alkyl-Het;
wherein each said alkyl is optionally substituted 1 or 2 times with substituents independently selected from R⁵³;
wherein each said phenyl and Het are optionally substituted 1 to 2 times with substituents independently selected from R⁵³, —O—(C₁₋₃)alkyl, —OH and (C₁₋₃)alkyl optionally substituted one time with Het or R⁵³;
R⁵³ is —COON, —NH₂, —NH(C₁₋₃)alkyl, —N((C₁₋₃)alkyl)₂ or —OH; and
Het is defined as a saturated or unsaturated 5- or 6-membered monocyclic ring system, including an aromatic ring system, containing 1 to 3 heteroatoms selected from N; and
p is 0 or 1.

11. The compound according to claim 1, or a pharmaceutically acceptable salt thereof, wherein X and Y are linked to form a 5-membered heteroaryl ring selected from: 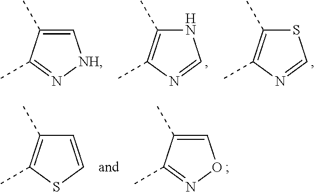
wherein said 5-membered heteroaryl ring is optionally substituted 1 to 2 times with substituents independently selected from (C₁₋₆)alkyl, (C₂₋₄)alkenyl, (C₂₋₄)alkynyl, (C₃₋₅)cycloalkyl, —(C₁₋₃)alkyl-(C₃₋₅)cycloalkyl, —(C₁₋₃)alkyl-Het, —(C₁₋₃)alkyl-phenyl, phenyl, —C(═O)—(C₁₋₃)alkyl, —C(═O)—NH₂, —C(═O)—N((C₁₋₃)alkyl)₂ and —SO₂—(C₁₋₃)alkyl;
wherein each said alkyl, phenyl and Het, either alone or in combination with another radical is optionally substituted 1 to 2 times with substituents independently selected from (C₁₋₃)alkyl, —O—(C₁₋₃)alkyl, OH, CN, COOH, halo, —(C₁₋₃)alkyl-Het, —NH₂, —N((C₁₋₃)alkyl)₂, —N(H)—C(═O)—O—(C₁₋₃)alkyl, —(C₁₋₃)alkyl-N(H)—(C₁₋₆)alkyl-O—(C₁₋₃)alkyl, —O-phenyl-C(═O)OH, —C(═O)—O—(C₁₋₃)alkyl —C(═O)NH₂, —C(═O)—N(H)—(C₁₋₃)alkyl-Het and —C(═O)—N(H)-Het; and
Het is defined as an unsaturated 5- or 6-membered monocyclic ring system including aromatic ring system containing one or more heteroatoms selected from N or O.

12. (canceled)

13. A pharmaceutical composition comprising a a compound of formula (I), or a pharmaceutically acceptable salt thereof, according to claim 1 and a pharmaceutically acceptable carrier.

14. (canceled)

15. A method for treating HIV which comprises administering to a host infected by HIV a therapeutically effective amount of a compound according to claim 1.
